(19) United States
(12) Patent Application Publication
(10) Pub. No.: US 2022/0023291 A1
(43) Pub. Date: Jan. 27, 2022

(54) COMPOSITIONS AND METHODS FOR REGULATING INFLAMMATION

(71) Applicant: University of Virginia Patent Foundation, Charlottesville, VA (US)

(72) Inventors: Dorian A. Rosen, Charlottesville, VA (US); Alban Gaultier, Charlottesville, VA (US)

(73) Assignee: University of Virginia Patent Foundation, Charlottesville, VA (US)

(21) Appl. No.: 17/311,624

(22) PCT Filed: Dec. 5, 2019
(86) PCT No.: PCT/US2019/064738

§ 371 Date: Jun. 7, 2021

Related U.S. Application Data
(60) Provisional application No. 62/775,416, filed on Dec. 5, 2018.

(51) Int. Cl. A61K 31/4748; A61P 29/00; A61K 38/54; A61K 31/495; A61K 31/438; A61K 31/5375; A61K 31/445; A61K 31/341; A61K 31/7088; A61K 45/06

(52) U.S. Cl. CPC A61K 31/4748; A61P 29/00; A61K 38/54; A61K 31/495; A61K 45/06; A61K 31/5375; A61K 31/445; A61K 31/341; A61K 31/7088; A61K 31/438

(57) ABSTRACT

Provided are methods for treating inflammation in subject in need thereof. In some embodiments, the methods include administering to the subject an effective amount of a Sigma-1 receptor (S1R) activity modulator to thereby treat inflammation in the subject. Also provided are pharmaceutical compositions that include an effective amount of a Sigma-1 receptor (S1R) activity modulator and uses of the pharmaceutical compositions to treat inflammation in a subject in need thereof.

### CROSS REFERENCE TO RELATED APPLICATION

The presently disclosed subject matter claims the benefit of U.S. Provisional Patent Application Ser. No. 62/775,416, filed Dec. 5, 2018; the disclosure of which is incorporated herein by reference in its entirety.

### GOVERNMENT INTEREST

This invention was made with government support under grant numbers NS083542, NS101281, and GM007055 awarded by The National Institutes of Health. The government has certain rights in the invention.

### BACKGROUND

Inflammation is the first arm of the immune response and is critical to combat infection. Inflammation is initiated when receptors present on innate immune cells recognize pathogen associated molecular patterns (PAMPs) and initiate a precise signaling response (Medzhitov, 2008). Lipopolysaccharide (LPS), a classical PAMP, binds to TLR4 and leads to the rapid activation and translocation of the transcription factor NF-κB to the nucleus, where it regulates the transient expression of pro-inflammatory cytokines IL-6 and TNF-α (Lu et al., 2008). Accurate orchestration of the inflammatory response is critical, since either impaired or excessive inflammation is associated with numerous pathologies in humans, including sepsis and auto-inflammatory disorders (Ruland, 2011).

Sepsis is an often-deadly complication of infection in which systemic inflammation damages the vasculature, leading to tissue hypoperfusion and multiple organ failure. Currently, the standard of care for sepsis is predominantly supportive, with few therapeutic options available. Because of increased sepsis incidence worldwide, there is an urgent need for discovery of novel therapeutic targets and development of new treatments. The recently discovered function of the endoplasmic reticulum (ER) in regulation of inflammation offers an underexplored avenue for sepsis control. Here, we identify the ER-resident protein Sigma-1 receptor (S1R) as an essential inhibitor of cytokine production in a preclinical model of septic shock. Mice lacking S1R succumb quickly to hypercytokinemia induced by a sub-lethal challenge in two models of acute inflammation. Mechanistically, we find that S1R restricts the endonuclease activity of the ER stress sensor IRE1 and cytokine expression, and does not inhibit the classical inflammatory signaling pathways. These findings could have significant clinical implications, as we further find that fluvoxamine, an anti-depressant therapeutic with high affinity for S1R, protects mice from lethal septic shock and dampens the inflammatory response in human blood leukocytes. Our data reveal the contribution of S1R to the restraint of the inflammatory response, and place S1R as a possible therapeutic target to treat bacterial-derived inflammatory pathology.

The ER, in addition to its protein synthesis and trafficking capabilities, has been shown to actively influence the inflammatory response to several different stimuli, including LPS, making this organelle a potential therapeutic target for control of inflammation (Martinon et al., 2010; Cho et al., 2013). Signaling in the ER occurs via three major pathways, initiated by distinct ER resident proteins: IRE1, PERK, and ATF6 (Hetz, 2012). Evolutionarily, the most conserved pathway is the IRE1 pathway, which has broad-reaching signaling capabilities, including the activation of inflammatory signaling programs. In particular, LPS specifically activates IRE1 through redox dysregulation (Martinon et al., 2010), leading to IRE1 mediated cleavage and degradation of mRNAs. Degraded mRNAs can activate inflammatory NF-κB signaling through association with RIG-I, though it is unclear whether this occurs during LPS-induced inflammation (Cho et al., 2013).

In addition to its role in the degradation of numerous mRNAs, active IRE1 specifically splices the mRNA of the transcription factor XBP1, leading to the removal of an intron and the production of active XBP1 (Chen & Brandizzi, 2013). In the context of inflammation, XBP1 transactivates numerous pro-inflammatory cytokines, including IL-6 and TNF-α (Martinon et al., 2010). While the role of IRE1 during inflammation is now evident, the mechanisms involved in the regulation of IRE1 function remain unclear. Sigma-1 receptor (S1R) is a ubiquitously expressed ER resident chaperone that associates with IRE1 during ER stress (Mori et al., 2013). S1R function is best studied in the central nervous system, where it has been shown to alter ion conductivity and improve cell survival (Kourrich et al., 2012). S1R has been implicated in the regulation of neurodegenerative diseases, including Alzheimer's disease and amyotrophic lateral sclerosis (Nguyen et al., 2015). Additionally, S1R has been shown to regulate the chemokine MCP-1 production in the CNS (Yao et al., 2010). Although S1R expression is not limited to the CNS, its function outside the brain remains poorly understood. As an ER resident chaperone, S1R is perfectly placed to detect ER dysbiosis, including the unfolded protein response. Since the markers of ER stress are commonly associated with inflammatory disorders (Yoshida, 2007; Morito & Nagata, 2012), S1R mediated regulation of ER function could be important during peripheral tissue inflammation, in addition to its activities in the CNS.

There is a long felt need in the art for compositions and methods useful for regulating inflammation. The presently disclosed subject matter satisfies these needs.

### SUMMARY

This Summary lists several embodiments of the presently disclosed subject matter, and in many cases lists variations and permutations of these embodiments of the presently disclosed subject matter. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter pertains to methods for treating inflammation in subject in need thereof. In some embodiments, the methods comprise administering to the subject an effective amount of a Sigma-1 receptor (S1R) activity modulator to thereby treat inflammation in the subject. In some embodiments, modulation of S1R activity treats conditions where an inflammatory response in a subject is detrimental or impaired. In some embodiments, the inflammation is associated with septic shock.

In some embodiments of the presently disclosed methods, the S1R activity modulator is a S1R agonist. In some embodiments, the S1R agonist is selected from the group consisting of wherein is a S1R agonist is selected from the group consisting of PRS-013, SA-4503, siramesine, (+)-pentazocine, (+)-SKF10,047, PRE084 (2-morpholin-4-ylethyl 1-phenylcyclohexane-1-carboxylate), SA4503 (1-[2-(3,4-dimethoxyphenyl)ethyl]-4-(3-phenylpropyl)piperazine), (±)-PPCC oxalate, PRE-084 hydrochloride, SA 4503 dihydrochloride, (+)-SK&F 10047 hydrochloride, and a compound commercially available under the following trade names ANAVEX® 2-73 (1-(2,2-diphenyltetrahydro-3-furanyl)-N,N-dimethylmethanamine), ANAVEX® 3-71 (1-(2,8-dimethyl-1-thia-3,8-diazaspiro(4.5)dec-3-yl)-3-(1H-indol-3-yl)propan-1-one), ANAVEX® 1-51, ANAVEX® 1079, ANAVEX® 1067, ANAVEX® 1037, ANAVEX® 1519, and ANAVEX® 1066.

In some embodiments of the presently disclosed methods, the S1R activity modulator is a composition that increases a level of S1R in the subject. In some embodiments, the composition that increases a level of S1R in the subject comprises an expression vector that expresses S1R. In some embodiments, the S1R activity modulator comprises an oligonucleotide, optionally an miRNA.

In some embodiments of the presently disclosed methods, two or more S1R activity modulators are administered in combination.

In some embodiments, the presently disclosed methods further comprise administering an additional therapeutic agent. In some embodiments, the additional therapeutic agent is an IRE1 specific endonuclease inhibitor. In some embodiments, the IRE1 specific endonuclease inhibitor is selected from the group consisting of 4μ8C (7-Hydroxy-4-methyl-2-oxo-2H-1-benzopyran-8-carboxaldehyde), STF 083010 (N-[(2-Hydroxy-1-naphthalenyl)methylene]-2-thiophenesulfonamide), MKC8866 (CAS #1338934-59-0), Kira 6 (CAS #1589527-65-0), Kira 8 (CAS #1630086-20-2), MKC3946 (CAS #1093119-54-0), GSK2850163 (CAS #2121989-91-9), 6-Bromo-2-hydroxy-3-methoxybenzaldehyde (CAS #20035-41-0), 3-methoxy-6-bromosalicylaldehyde salicylaldimines, toyocamycin, N9-(3-(dimethylamino) propyl)-N3,N3,N6,N6-tetramethylacridine-3,6,9-triamine (3,6-DMAD), Hydroxy-aryl-aldehydes (HAA), and irestatin.

In some embodiments, the presently disclosed subject matter pertains to uses of a pharmaceutical composition comprising, consisting essentially of, or consisting of an effective amount of a Sigma-1 receptor (S1R) activity modulator to treat inflammation in a subject in need thereof.

In some embodiments, the presently disclosed subject matter pertains to use of an effective amount of a Sigma-1 receptor (S1R) activity modulator for the preparation of a medicament to treat inflammation in a subject in need thereof.

In some embodiments, the presently disclosed subject matter pertains to pharmaceutical compositions comprising, consisting essentially of, or consisting of an effective amount of a Sigma-1 receptor (S1R) activity modulator to treat inflammation in a subject in need thereof.

In some embodiments of the uses and/or compositions disclosed herein, modulation of S1R activity treats conditions where an inflammatory response in a subject is detrimental or impaired.

In some embodiments of the uses and/or compositions disclosed herein, the inflammation is associated with septic shock.

In some embodiments of the uses and/or compositions disclosed herein, the S activity modulator is a S1R agonist. In some embodiments, the S1R agonist is selected from the group consisting of PRS-013, SA-4503, siramesine, (+)-pentazocine, (+)-SKF10,047, PRE084 (2-morpholin-4-ylethyl 1-phenylcyclohexane-1-carboxylate), SA4503 (1-[2-(3,4-dimethoxyphenyl)ethyl]-4-(3-phenylpropyl)piperazine), (±)-PPCC oxalate, PRE-084 hydrochloride, SA 4503 dihydrochloride, (+)-SK&F 10047 hydrochloride, and a compound commercially available under the following trade names ANAVEX® 2-73, ANAVEX® 3-71, ANAVEX® 1-51, ANAVEX® 1079, ANAVEX® 1067, ANAVEX® 1037, ANAVEX® 1519, and ANAVEX® 1066.

In some embodiments of the uses and/or compositions disclosed herein, the S1R activity modulator is a composition that increases a level of S1R in the subject.

In some embodiments of the uses and/or compositions disclosed herein, the composition that increases a level of S1R in the subject comprises an expression vector that expresses S1R.

In some embodiments of the uses and/or compositions disclosed herein, the S1R activity modulator comprises an oligonucleotide.

In some embodiments of the uses and/or compositions disclosed herein, two or more S1R activity modulators are provided in combination.

In some embodiments, the uses and/or compositions disclosed herein further comprise providing an additional therapeutic agent. In some embodiments, the additional therapeutic agent is an IRE1 specific endonuclease inhibitor. In some embodiments, the IRE1 specific endonuclease inhibitor is selected from the group consisting of 4μ8C (7-Hydroxy-4-methyl-2-oxo-2H-1-benzopyran-8-carboxaldehyde), STF 083010 (N-[(2-Hydroxy-1-naphthalenyl)methylene]-2-thiophenesulfonamide), MKC8866 (CAS #1338934-59-0), Kira 6 (CAS #1589527-65-0), Kira 8 (CAS #1630086-20-2), MKC3946 (CAS #1093119-54-0), GSK2850163 (CAS #2121989-91-9), 6-Bromo-2-hydroxy-3-methoxybenzaldehyde (CAS #20035-41-0), 3-methoxy-6-bromosalicylaldehyde salicylaldimines, toyocamycin, N9-(3-(dimethylamino) propyl)-N3,N3,N6,N6-tetramethylacridine-3,6,9-triamine (3,6-DMAD), Hydroxy-aryl-aldehydes (HAA), and irestatin.

Accordingly, it is an object of the presently disclosed subject matter to provide compositions and methods for modulating S1R biological activity. This and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Figures, and EXAMPLES. Additionally, various aspects and embodiments of the presently disclosed subject matter are described in further detail below.

### BRIEF DESCRIPTION OF THE FIGURES

FIGS. 1A-1D: Lack of S1R led to increased mortality and hypercytokinemia in endotoxin challenge. FIG. 1A is a Kaplan-Meier survival plot of mice challenged with 5 mg/kg of LPS (WT n=11. KO n=13). Statistical significance was calculated by log-rank test. FIGS. 1B and 1C are plots of serum concentrations of TNF-α (FIG. 1B) and IL-6 (FIG. 1C) in mice acutely treated with 5 mg/kg LPS determined by ELISA. Each symbol represents one mouse. Statistical significance was calculated by Student's t-test. FIG. 1D are representative H&E stained liver sections of mice that succumbed to endotoxin challenge. * indicates p<0.05, ** indicates p<0.01.

FIGS. 2A-2F: S1R KO BMDM produced unusually high levels of pro-inflammatory cytokines. FIG. 2A is a Western blot for S1R in unstimulated BMDM. FIGS. 2B-2D are bar graphs of expression of IL-6 (FIG. 2B), IL-1β (FIG. 2C), and IL-10 (FIG. 2D) measured by qPCR in BMDM stimulated for 6 hours with <1% H₂O (NT) or 100 ng/mL LPS in culture medium. FIG. 2E is a bar graph of secretion of IL-6 determined by ELISA in BMDM stimulated for 6 hours with 1 μg/mL LPS in culture medium. Data in FIGS. 2A-2E are representative of n=3. Statistical significance was calculated by one-way ANOVA with post-hoc Tukey test. FIG. 2F is a series of plots of secretion of several pro-inflammatory cytokines by BMDM stimulated for 6 hours with 1 μg/mL LPS was measured by LUMINEX® multiplex assay. Supernatant IL-6 concentrations in FIGS. 2E and 2F were normalized to amount of protein in each well to account for differences in cell death or proliferation between genotypes. Each symbol represents BMDM from one mouse. *** indicates p<0.001.

FIGS. 3A-3F: Increased NF-κB activity does not drive inflammation in S1R KO BMDM. FIGS. 3A-3C are immunoblots of lysates of BMDM treated with 100 ng/mL LPS in culture medium for indicated durations. Lysates were analyzed by immunoblotting for phosphorylated and total p65 NF-κB (FIG. 3A), phosphorylated and total ERK1/2 (FIG. 3B), and phosphorylated and total JNK (FIG. 3C). FIG. 3D is a series of representative immunofluorescence images of BMDM stained for total p65 NF-κB either basally (NT) or after 45 minute exposure to 100 ng/mL LPS in culture medium. FIG. 3E is a bar graph showing quantification of IF for amount of p65 NF-κB nuclear localization in BMDM after LPS treatment. Scale bar=50 FIG. 3F is a bar graph showing secretion of IL-6 measured by ELISA in BMDM that were stimulated with 1 μg/mL LPS in culture medium for 6 hours, in the presence of <1% DMSO, 20 μM JSH-23, 20 μM PD98059, or 1 μM SP600125. Supernatant IL-6 concentrations were normalized to amount of protein in each well to account for differences in cell death or proliferation between genotypes. Statistical significance in FIGS. 3E and 3F was calculated by one-way ANOVA with post-hoc Tukey test. All data are representative of n=2-3. ** indicates p<0.01, *** indicates p<0.001.

FIGS. 4A-4H: IRE1 activation is increased in S1R KO BMDM and is associated with elevated Bax. FIG. 4A is a plot of XBP1 splicing ratio in peritoneal cells acutely isolated from wild-type mice 3 hours after intraperitoneal LPS challenge at a dose of 2 mg/kg. Each symbol indicates one mouse. Statistical significance was calculated by unpaired t-test. FIG. 4B is a bar graph of XBP1 splicing ratio in BMDM stimulated for 6 hours with <1% DMSO, 100 ng/mL LPS, and/or 5 μM 4μ8C in culture medium. FIG. 4C is a bar graph of XBP1 splicing ratio in BMDM stimulated for 6 hours with <1% DMSO or 5 μM tunicamycin in culture medium. XBP1 splicing in FIGS. 4A-4C was measured by qPCR, where splicing ratio indicates the amount of spliced XBP1 transcript divided by the amount of unspliced XBP1 transcript. FIG. 4D is an immunoblot for phosphorylated and total IRE1 in BMDM treated with 100 ng/mL LPS for the indicated durations. FIGS. 4E and 4F are immunoblots for Bax (FIG. 4E) or Bcl-2 (FIG. 4F) in BMDM treated for 3 hours with 100 ng/mL LPS in culture medium. FIG. 4G is densitometric quantification of Bax abundance in BMDM. Per condition, each symbol represents BMDM from one mouse. Statistical significance was calculated by paired t-test. FIG. 4H is a plot of the ratio of protein abundance of Bax vs. Bcl-2, as calculated by western blot densitometry. Data in FIGS. 4B-4F are representative of n=3. ** indicates p<0.01, *** indicates p<0.001.

FIGS. 5A-5D: S1R controls IRE1 abundance post-transcriptionally. FIG. 5A is a bar graph of qPCR for IRE1 expression in BMDM treated with <1% DMSO or 5 μM 4μ8C in culture medium. Statistical significance was calculated by one-way ANOVA with post-hoc Tukey test. FIG. 5B is an immunoblot for total IRE1, S1R, and FLAG in HEK293 cells that overexpress IRE1 and empty vector (EV), MESD-FLAG (MESD), or S1R. FIG. 5C is a graph of quantitation of total IRE1 and actin in BMDM treated with 5 μg/mL cycloheximide (CHX) to block protein translation. FIG. 5D is an immunoblot for total IRE1, S1R, and FLAG in HEK293 cells that overexpressed IRE1 and empty vector (EV), MESD-FLAG (MESD), or S1R, and treated with <1% DMSO, 5 μM lactacystin, or 30 μM chloroquine for 6 hours. All data are representative of n=3. ** indicates p<0.01.

FIGS. 6A-6F: Excessive cytokine production in S1R KO BMDM is dependent on LPS induction of IRE1 endonuclease activity. FIGS. 6A and 6B are bar graphs of qPCR for expression of the pro-inflammatory cytokines IL-6 (FIG. 6A) and IL-1β (FIG. 6B) in BMDM treated with <1% DMSO, 100 ng/mL LPS, and/or 5 μM 4μ8C, as indicated, for 6 hours. FIGS. 6C and 6D are bar graphs of IL-6 secretion normalized to amount of protein in each well of BMDM (FIG. 6C) or primary lung fibroblasts (FIG. 6D) treated with 1 μg/mL LPS and <1% DMSO or 5 μM 4μ8C for 6 hours. FIGS. 6E and 6F are bar graphs of BMDM treated with media conditioned by BMDM as indicated for 6 hours, and expression of IL-6 (FIG. 6E) or IL-1β (FIG. 6F) measured by qPCR. All data are representative of n=3. Statistical significance was calculated by one-way ANOVA with post-hoc Tukey test. ** indicates p<0.01, *** indicates p<0.001.

FIGS. 7A-7E: Inhibition of IRE1 rescues S1R KO mice from mortality during in vivo endotoxin challenge. FIG. 7A is a bar graph of BMDM treated for 6 hours with 1 μg/mL LPS and 1% DMSO (vehicle), 4μ8C (5 μM), or STF 083010 (60 μM). IL-6 concentration was determined by ELISA and normalized to the amount of protein present in each well. Statistical significance was calculated using one-way ANOVA with post-hoc Tukey test. Data are representative of n=3. FIG. 7B is a plot of IL-6 concentration measured by ELISA from mice challenged with LPS (2 mg/kg) and treated with STF 083010 (30 mg/kg) or vehicle (33% Kolliphor-EL in saline) control at 0 and 24 hours post LPS injection. Serum was collected 3 hours after LPS injection. Each symbol represents one mouse. FIG. 7C is a plot of wild type mice treated with LPS (2 mg/kg) and 9 mL/kg of saline or 33% Kolliphor-EL in saline. IL-6 in serum and peritoneal wash 3 hours after LPS injection was measured by ELISA. Each symbol represents one mouse. FIG. 7D is a Kaplan-Meier survival plot of mice challenged with LPS as in FIG. 7B. n=8-9 mice per group. Statistical significance was determined by log-rank test. FIG. 7E is a plot of ELISA for IL-6 in peritoneal wash three hours after mice were challenged with LPS as in FIG. 7B. Each symbol represents one mouse. Statistical significance in FIGS. 7B, 7C, and 7E was calculated by student's t-test. * indicates p<0.05, *** indicates p<0.001.

FIGS. 8A-8F: S1R is an inhibitor of IRE1 during inflammation. FIG. 8A is a schematic experimental design and principle of proximity ligation assay. HA: Hemagglutinin. FIG. 8B is a pair of Western blots on input lysates and biotinylated (streptavidin pulldown) proximity ligation samples of HEK293 transfected with BirA or S1R-BirA, then challenged for 24 hours with 100 ng/mL LPS in the presence of 80 μM biotin. FIG. 8C is a bar graph of densitometric quantification of FIG. 8B (n=4, *p<0.05, repeated measures one-way ANOVA with post-hoc Sidak test). FIG. 8D is a schematic depicting activity modulators of IRE1 as disclosed herein. XBP1 (US): Unspliced XBP1 transcript; XBP1 (S): Spliced XBP1 transcript. FIG. 8E is a graph of XBP1 splicing ratio (i.e., GAPDH-normalized spliced XBP1 transcript/GAPDH-normalized unspliced XBP1 transcript) in BMDM stimulated for 6 hours with DMSO, 100 ng/mL LPS, or 100 ng/mL LPS+IRE1 inhibitor (5 μM 4μ8C; n=3; n.s.: not significant; *p<0.05, two-way ANOVA with post-hoc Sidak test). FIG. 8F is a graph of XBP1 splicing ratio in BMDM stimulated with DMSO or an activator of IRE1 for 6 hours (n=3, each dot represents one individual experiment, n.s. not significant, two-way ANOVA with post-hoc Sidak test).

FIGS. 9A-9M: S1R controls the production of inflammatory cytokines by inhibiting IRE1. FIG. 9A is a bar graph of qPCR on BMDM stimulated for 6 hours with vehicle (NT) or 100 ng/mL LPS (representative of n=4 independent experiments; **p<0.01, two-way ANOVA with post-hoc Tukey test). FIG. 9B is a bar graph of IL-6 ELISA on supernatant from BMDM stimulated for 6 hours with 1 μg/mL LPS (representative of n=4 independent experiments; ***p<0.001, t-test). FIGS. 9C and 9D are bar graphs of pro-IL-1β or IL-10 expression measured by qPCR in BMDM stimulated for 6 hours with vehicle (NT) or 100 ng/mL LPS (representative of n=3 independent experiment is shown, n.s. not significant, ***p<0.001, two-way ANOVA with post-hoc Tukey test). FIG. 9E is a graph of qPCR on TLR4-MD2-CD14 expressing HEK293 cells that were transfected with empty vector (EV) or S1R and stimulated for 6 hours with 100 ng/mL LPS (n=3, each dot pair represents one independent experiment, *p<0.05, paired t-test). FIG. 9F is a bar graph of qPCR on BMDM stimulated for 6 hours with vehicle (DMSO), 100 ng/mL LPS, or 100 ng/mL LPS+IRE1 inhibitor (5 μM 4μ8C; representative of n=3; n.s.: not significant, ***p<0.001, two-way ANOVA with post-hoc Tukey test). FIG. 9G is an immunoblot of BMDM treated with vehicle (control) or 100 ng/mL LPS for 6 hours and lysates for the indicated proteins (representative of n=3 independent experiments). FIGS. 9H and 9I are flow cytometric assessments of surface TLR4 on CD45+ CD11b+ F4/80+ BMDM (n=3 mice/group, each dot represents one mouse, n.s. not significant, t-test). FIGS. 9J and 9K are immunoblots of BMDM treated with 100 ng/mL LPS for indicated durations and lysates analyzed by immunoblotting for the indicated proteins (representative of n=3 independent experiments). FIG. 9L is a bar graph of IL-6 ELISA on supernatant from BMDM stimulated for 6 hours with 1 μg/mL LPS with either <1% DMSO, NF-κB inhibitor (20 μM JSH-23), ERK inhibitor (20 μM PD98059), JNK inhibitor (1 μM SP600125), or IRE1 inhibitor (5 μM 4μ8C) (representative of n=3, n.s. not significant, **p<0.01, ***p<0.001, two-way ANOVA with post-hoc Tukey test). FIG. 9M is plots of the gating strategy and quantification of immunophenotyping on blood. N=4, each dot represents data from one animal, all comparisons were not significant, t-test.

FIGS. 10A-10R: S1R is protective in two murine models of septic shock. FIG. 10A is a schematic of an exemplary experimental design. FIG. 10B is a survival curve of WT and S1R KO mice following LPS administration (n=11-13 mice/group, LPS=5 mg/kg, **p<0.01, log-rank test). FIG. 10C is a graph of ELISA for TNF-α in serum 1.5 hours after LPS injection (each dot represents one mouse, *p<0.05, t-test). FIG. 10D is a graph of ELISA for IL-6 in serum collected 3 hours after LPS injection (each dot represents one mouse, *p<0.05, t-test). FIGS. 10E and 10F are plots of gating strategy and quantification of immunophenotyping on peritoneal contents, respectively. CD11c+ gate was drawn on CD11c FMO. n=4, each dot represents data from one animal, all comparisons were not significant, t-test. FIGS. 10G-10I are plots of gating strategy and quantification, respectively, of immunophenotyping on spleen and lymph node. CD11c+ gate was drawn on CD11c FMO. n=4, each dot represents data from one animal, all comparisons were not significant, t-test. FIG. 10J is a bar graph of qPCR analysis for XBP1 splicing ratio (i.e., GAPDH-normalized spliced XBP1 transcript/GAPDH-normalized unspliced XBP1 transcript) in primary lung fibroblasts stimulated for 6 hours with 100 ng/mL LPS and vehicle (DMSO) or IRE1 inhibitor (5 μM 4μ8C; representative of N=3 independent experiments). FIG. 10K is a bar graph of ELISA on supernatant from primary lung fibroblasts stimulated for 6 hours with 1 μg/mL LPS and vehicle (<1% DMSO) or IRE1 inhibitor (5 μM 4μ8C), normalized to total protein in well (representative of n=3 independent experiment is shown, ***p<0.001, two-way ANOVA with post-hoc Tukey test). FIG. 10L is a survival curve of WT and S1R KO mice following administration of fecal content (n=10-13 mice/group, fecal slurry=1 g/kg, *p<0.05, log-rank test). FIG. 10M is a graph of ELISA for IL-6 in serum collected 3 hours after fecal slurry injection (each dot represents one mouse, *p<0.05, t-test). FIG. 10N is a graph of rectal temperatures of animals presented in FIG. 10L (n=10-13 mice/group, **p<0.01, ***p<0.001, two-way repeated measures ANOVA with post-hoc Sidak test). FIGS. 10O-10R are graphs showing the results of mice injected with 5 mg/kg LPS or 1 mg/kg fecal slurry and, 24 hours later, serum was analyzed for amount of alanine aminotransferase (ALT; FIG. 10O), aspartate aminotransferase (AST; FIG. 10P), creatinine (FIG. 10Q), and and creatine kinase (CK; FIG. 10R). Each dot represents one mouse (n=10-12 per group. *p<0.05, **p<0.01, ***p<0.001, two-way ANOVA).

FIGS. 11A-11H: Pharmacological modulation of S1R and IRE1 function in sepsis models. FIG. 11A is a graph of XBP1 splicing on liver homogenate from mice challenged with 5 mg/kg LPS for 3 hours. Data shown are ratio of XBP1 spliced transcript/XBP1 unspliced transcript (each dot represents one mouse, **p<0.01, two-way ANOVA with post-hoc Sidak test). FIG. 11B is a schematic of an exemplary experimental design. FIG. 11C is a bar graph of ELISA on supernatant from BMDM stimulated for 6 hours with 1 μg/mL LPS and vehicle (<1% DMSO) or two IRE1 inhibitors (5 μM 4μ8C or 20 μM STF), representative of n=3 independent experiment is shown, ***p<0.001, two-way ANOVA with post-hoc Tukey test). FIG. 11D is a survival curve of WT and S1R deficient mice treated with vehicle (33% Kolliphor in saline) or STF (30 mg/kg) following administration of LPS as indicated in FIG. 11B (n=15-16 mice/group, LPS=2 mg/kg, **p<0.01, ***p<0.001, log-rank test). FIG. 11E is a graph of IL-6 peritoneal exudate 3 hours after LPS injection in mice (each dot represents one mouse, *p<0.05, two-way ANOVA with post-hoc Sidak test). FIG. 11F is a graph of ELISA on serum from mice challenged with 2 mg/kg LPS and 33% Kolliphor (vehicle) or 30 mg/kg of the IRE1 inhibitor (STF) as in FIG. 10P. n=9-11 mice/group, each dot represents one mouse. Genotype-treatment interaction *p<0.05, two-way ANOVA). FIG. 11G is a graph of ELISA for IL-6 in serum of WT mice treated with 9 μL/g of 33% Kolliphor in saline (Kolliphor) or an equivalent volume of saline (n=5-6 mice/group, each dot represents one mouse, **p<0.001, t-test). FIG. 11H is a survival curve of Saline+33% Kolliphor, no LPS, controls, challenged as in FIG. 11D (n=6-7, n.s., log-rank test).

FIGS. 12A-12O: Therapeutic administration of the S1R agonist fluvoxamine is protective during models of sepsis. FIG. 12A is a schematic of an exemplary experimental design. FIG. 12B is a survival curve of WT and S1R deficient mice treated with vehicle or fluvoxamine (20 mg/kg) following administration of LPS as indicated in E (n=13-17 mice/group, LPS=6 mg/kg *p<0.05, **p<0.01, log-rank test). FIG. 12C is a graph of serum IL-6 ELISA 3 hours after LPS injection in mice challenged as shown in E (each dot represents one mouse, n. s. not significant, **p<0.01, two-way ANOVA with post-hoc Sidak test. Genotype-treatment interaction: *p<0.05, two-way ANOVA). FIGS. 12D and 12E are exemplary experimental designs for LPS challenge (FIG. 12D) or FIP with therapeutic S1R agonist treatment (FIG. 12E). FIG. 12F is a graph of rectal temperatures of mice measured immediately before LPS injection and one hour after (each dot represents one mouse, ***p<0.001, paired t-test). FIG. 12G is a graph of rectal temperatures of mice measured immediately before FIP induction and 0.5 hour after (each dot represents one mouse, ***p<0.001, paired t-test). FIG. 12H is a graph of clinical scores, expressed as total murine sepsis score, of mice treated as in FIG. 12D, **p<0.01, ***p<0.001, two-way ANOVA with post-hoc Sidak test. FIG. 12I is a graph of clinical scores, expressed as total murine sepsis score, of mice treated as in B, (n=14 mice/group, ***p<0.001, two-way ANOVA with post-hoc Sidak test). FIG. 12J is a graph of rectal temperatures of mice 24 hours after I.P. LPS injection, treated with saline vehicle or fluvoxamine (FLV) as indicated in A (each dot represents one mouse, ***p<0.05, t-test). FIG. 12K is a survival curve of mice challenged with 6 mg/kg LPS and given therapeutic FLV or saline as indicated in FIG. 12D (n=16-20, ***p<0.001, log-rank test). FIG. 12L is a graph of rectal temperatures of mice 24 hours after FIP induction, treated with saline vehicle or FLV as indicated in FIG. 12E (each dot represents one mouse, **p<0.01, t-test). FIG. 12M is a survival curve of mice challenged with 1.5 g/kg fecal slurry and given therapeutic FLV or saline as indicated in FIG. 12L (n=14 mice/group **p<0.01, log-rank test). FIG. 12N is an exemplary experimental design for FIP challenge with FLV and the antibiotic ceftriaxone (CRO) treatment. FIG. 12O is a survival of C57/B16 mice treated with fluvoxamine (20 mg/kg) i.p. and/or ceftriaxone (100 mg/kg s.c.) following administration of fecal slurry at 1.5 g/kg (n=10-12 mice/group, **p<0.01, ***p<0.001, log-rank test).

FIGS. 13A-13D: The S1R agonist fluvoxamine is anti-inflammatory in human tissue. FIGS. 13A-13D are graphs of multiplex ELISA on serum from human blood. Heparinized whole blood was stimulated ex vivo with 10 ng/mL LPS and vehicle (RPMI) or 20 μM FLV for 4 hours (n=4, each dot pair represents serum from one participant, *p<0.05, **p<0.01, paired t-test).

FIG. 14 is a series of plots of multiplex ELISA on serum from heparinized human blood stimulated ex vivo with 10 ng/mL LPS and vehicle (RPMI) or 20 μM fluvoxamine for 4 hours (n=4, each dot pair represents serum from one participant, paired t-test).

FIG. 15 is a schematic of a proposed mechanism of action of S1R during LPS-mediated inflammatory response. FLV: fluvoxamine; XBP1 (US): Unspliced XBP1 transcript; XBP1 (S) Spliced XBP1 transcript.

### DETAILED DESCRIPTION

Headings are included herein for reference and to aid in locating certain sections. These headings are not intended to limit the scope of the concepts described therein under, and these concepts may have applicability in other sections throughout the entire specification.

The endoplasmic reticulum (ER) is classically defined as the site of secreted protein synthesis and trafficking. Recently, the ER functions have been extended to a plethora of new biological roles, including inflammation. Activation of the ER stress sensor IRE1 is essential for the normal inflammatory response to stimuli such as LPS. However, the mechanisms by which IRE1 regulates inflammation remain unclear. Disclosed herein is the novel observation of a role of the ER protein Sigma-1 receptor (S1R) as a critical inhibitor of LPS-induced cytokine production. Mice lacking S1R succumb quickly to hypercytokinemia after administration of a sub-lethal dose of LPS. Mechanistically, S1R controls IRE1 endonuclease activity required for cytokine expression and regulates the biosynthesis of IRE1, without an impact on cytosolic inflammatory signaling pathways. Data disclosed herein reveal the contribution of S1R to the restraint of the inflammatory response, and place S1R as a therapeutic target to treat inflammatory disorders.

### I. Abbreviations and Acronyms

ER: endoplasmic reticulum

FLV: fluvoxamine

IRE1: Inositol-requiring enzyme 1, an ER resident signaling protein

LPS: Lipopolysaccharide

MAM: mitochondrial-associated membrane

PAMPs: pathogen associated molecular patterns

S1R: Sigma-1 receptor

STF: STF 083010

### II. Definitions

In describing and claiming the presently disclosed subject matter, the following terminology will be used in accordance with the definitions set forth below.

### II.A. General Definitions

The articles “a” and “an” are used herein to refer to one or to more than one (i.e., to at least one) of the grammatical object of the article. By way of example, “an element” means one element or more than one element.

The term “about”, as used herein, means approximately, in the region of, roughly, or around. When the term “about” is used in conjunction with a numerical range, it modifies that range by extending the boundaries above and below the numerical values set forth. For example, In some embodiments, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

The terms “additional therapeutically active compound” or “additional therapeutic agent”, as used in the context of the presently disclosed subject matter, refers to the use or administration of a compound for an additional therapeutic use for a particular injury, disease, or disorder being treated. Such a compound, for example, could include one being used to treat an unrelated disease or disorder, or a disease or disorder which may not be responsive to the primary treatment for the injury, disease or disorder being treated. Disease and disorders being treated by the additional therapeutically active agent include, for example, hypertension and diabetes. The additional compounds may also be used to treat symptoms associated with the injury, disease, or disorder, including, but not limited to, pain and inflammation.

The term “adult” as used herein, is meant to refer to any non-embryonic or non-juvenile subject. For example, the term “adult adipose tissue stem cell”, refers to an adipose stem cell, other than that obtained from an embryo or juvenile subject.

As used herein, an “agonist” is a composition of matter which, when administered to a mammal such as a human, enhances or extends a biological activity attributable to the level or presence of a target compound or molecule of interest in the subject.

A disease or disorder is “alleviated” if the severity of a symptom of the disease, condition, or disorder, or the frequency with which such a symptom is experienced by a subject, or both, are reduced.

As used herein, amino acids are represented by the full name thereof, by the three letter code corresponding thereto, or by the one-letter code corresponding thereto, as indicated in Table 1:

TABLE 1
Amino Acid Codes and Functionally Equivalent Codons

| | 3-Letter | 1-Letter | |
|---|---|---|---|
| Full Name | Code | Code | Functionally Equivalent Codons |
| Aspartic Acid | Asp | D | GAC; GAU |
| Glutamic Acid | Glu | E | GAA; GAG |
| Lysine | Lys | K | AAA; AAG |
| Arginine | Arg | R | AGA; AGG; CGA; CGC; CGG; |
| | | | CGU |
| Histidine | His | H | CAC; CAU |
| Tyrosine | Tyr | Y | UAC; UAU |
| Cysteine | Cys | C | UGC; UGU |
| Asparagine | Asn | N | AAC; AAU |
| Glutamine | Gln | Q | CAA; CAG |
| Serine | Ser | S | ACG; AGU; UCA; UCC; UCG; |
| | | | UCU |
| Threonine | Thr | T | ACA; ACC; ACG; ACU |
| Glycine | Gly | G | GGA; GGC; GGG; GGU |
| Alanine | Ala | A | GCA; GCC; GCG; GCU |
| Valine | Val | V | GUA; GUC; GUG; GUU |
| Leucine | Leu | L | UUA; UUG; CUA; CUC; CUG; |
| | | | CUU |
| Isoleucine | Ile | I | AUA; AUC; AUU |
| Methionine | Met | M | AUG |
| Proline | Pro | P | CCA; CCC; CCG; CCU |
| Phenylalanine | Phe | F | UUC; UUU |
| Tryptophan | Trp | W | UGG |

The expression “amino acid” as used herein is meant to include both natural and synthetic amino acids, and both D and L amino acids. “Standard amino acid” means any of the twenty standard L-amino acids commonly found in naturally occurring peptides. “Nonstandard amino acid residue” means any amino acid, other than the standard amino acids, regardless of whether it is prepared synthetically or derived from a natural source. As used herein, “synthetic amino acid” also encompasses chemically modified amino acids, including but not limited to salts, amino acid derivatives (such as amides), and substitutions. Amino acids contained within the peptides of the presently disclosed subject matter, and particularly at the carboxy- or amino-terminus, can be modified by methylation, amidation, acetylation or substitution with other chemical groups which can change the peptide's circulating half-life without adversely affecting their activity. Additionally, a disulfide linkage may be present or absent in the peptides of the presently disclosed subject matter.

The term “amino acid” is used interchangeably with “amino acid residue”, and may refer to a free amino acid and to an amino acid residue of a peptide. It will be apparent from the context in which the term is used whether it refers to a free amino acid or a residue of a peptide.

Amino acids may be classified into seven groups on the basis of the side chain R: (1) aliphatic side chains, (2) side chains containing a hydroxylic (OH) group, (3) side chains containing sulfur atoms, (4) side chains containing an acidic or amide group, (5) side chains containing a basic group, (6) side chains containing an aromatic ring, and (7) proline, an imino acid in which the side chain is fused to the amino group.

Amino acids have the following general structure:

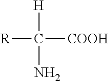

The nomenclature used to describe the peptide compounds of the presently disclosed subject matter follows the conventional practice wherein the amino group is presented to the left and the carboxy group to the right of each amino acid residue. In the formulae representing selected specific embodiments of the presently disclosed subject matter, the amino- and carboxy-terminal groups, although not specifically shown, will be understood to be in the form they would assume at physiologic pH values, unless otherwise specified.

The term “basic” or “positively charged” amino acid, as used herein, refers to amino acids in which the R groups have a net positive charge at pH 7.0, and include, but are not limited to, the standard amino acids lysine, arginine, and histidine.

As used herein, an “analog” of a chemical compound is a compound that, by way of example, resembles another in structure but is not necessarily an isomer (e.g., 5-fluorouracil is an analog of thymine).

An “antagonist” is a composition of matter which when administered to a mammal such as a human, inhibits a biological activity attributable to the level or presence of a compound or molecule of interest in the subject.

The term “antibody”, as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the presently disclosed subject matter may exist in a variety of forms including, for example, polyclonal antibodies, monoclonal antibodies, Fv, Fab and F(ab)₂, as well as single chain antibodies and humanized antibodies.

The term “antimicrobial agents” as used herein refers to any naturally-occurring, synthetic, or semi-synthetic compound or composition or mixture thereof, which is safe for human or animal use as practiced in the methods of the presently disclosed subject matter, and is effective in killing or substantially inhibiting the growth of microbes. “Antimicrobial” as used herein, includes antibacterial, antifungal, and antiviral agents.

As used herein, the term “antisense oligonucleotide” or antisense nucleic acid means a nucleic acid polymer, at least a portion of which is complementary to a nucleic acid which is present in a normal cell or in an affected cell. “Anti sense” refers particularly to the nucleic acid sequence of the non-coding strand of a double stranded DNA molecule encoding a protein, or to a sequence which is substantially homologous to the non-coding strand. As defined herein, an anti sense sequence is complementary to the sequence of a double stranded DNA molecule encoding a protein. It is not necessary that the antisense sequence be complementary solely to the coding portion of the coding strand of the DNA molecule. The antisense sequence may be complementary to regulatory sequences specified on the coding strand of a DNA molecule encoding a protein, which regulatory sequences control expression of the coding sequences. The antisense oligonucleotides of the presently disclosed subject matter include, but are not limited to, phosphorothioate oligonucleotides and other modifications of oligonucleotides.

The term “autologous”, as used herein, refers to something that occurs naturally and normally in a certain type of tissue or in a specific structure of the body. In transplantation, it refers to a graft in which the donor and recipient areas are in the same individual, or to blood that the donor has previously donated and then receives back, usually during surgery.

The term “basal medium”, as used herein, refers to a minimum essential type of medium, such as Dulbecco's Modified Eagle's Medium, Ham's F12, Eagle's Medium, RPMI, ARB, etc., to which other ingredients may be added. The term does not exclude media which have been prepared or are intended for specific uses, but which upon modification can be used for other cell types, etc.

The term “biocompatible”, as used herein, refers to a material that does not elicit a substantial detrimental response in the host.

The term “biodegradable”, as used herein, means capable of being biologically decomposed. A biodegradable material differs from a non-biodegradable material in that a biodegradable material can be biologically decomposed into units which may be either removed from the biological system and/or chemically incorporated into the biological system.

The term “biological sample”, as used herein, refers to samples obtained from a living organism, including skin, hair, tissue, blood, plasma, cells, sweat, and urine.

The term “bioresorbable”, as used herein, refers to the ability of a material to be resorbed in vivo. “Full” resorption means that no significant extracellular fragments remain. The resorption process involves elimination of the original implant materials through the action of body fluids, enzymes, or cells. Resorbed calcium carbonate may, for example, be redeposited as bone mineral, or by being otherwise re-utilized within the body, or excreted. “Strongly bioresorbable”, as the term is used herein, means that at least 80% of the total mass of material implanted is resorbed within one year.

The phrases “cell culture medium”, “culture medium” (plural “media” in each case), and “medium formulation” refer to a nutritive solution for cultivating cells and may be used interchangeably.

The term “clearance”, as used herein refers to the physiological process of removing a compound or molecule, such as by diffusion, exfoliation, removal via the bloodstream, and excretion in urine, or via sweat or other fluid.

A “control” cell, tissue, sample, or subject is a cell, tissue, sample, or subject of the same type as a test cell, tissue, sample, or subject. The control may, for example, be examined at precisely or nearly the same time the test cell, tissue, sample, or subject is examined. The control may also, for example, be examined at a time distant from the time at which the test cell, tissue, sample, or subject is examined, and the results of the examination of the control may be recorded so that the recorded results may be compared with results obtained by examination of a test cell, tissue, sample, or subject. The control may also be obtained from another source or similar source other than the test group or a test subject, where the test sample is obtained from a subject suspected of having a disease or disorder for which the test is being performed.

A “test” cell, tissue, sample, or subject is one being examined or treated.

A “pathoindicative” cell, tissue, or sample is one which, when present, is an indication that the animal in which the cell, tissue, or sample is located (or from which the tissue was obtained) is afflicted with a disease or disorder. By way of example, the presence of one or more breast cells in a lung tissue of an animal is an indication that the animal is afflicted with metastatic breast cancer.

A tissue “normally comprises” a cell if one or more of the cell are present in the tissue in an animal not afflicted with a disease or disorder.

A “compound”, as used herein, refers to any type of substance or agent that is commonly considered a drug, or a candidate for use as a drug, combinations, and mixtures of the above, as well as polypeptides and antibodies of the presently disclosed subject matter.

“Cytokine”, as used herein, refers to intercellular signaling molecules, the best known of which are involved in the regulation of mammalian somatic cells. A number of families of cytokines, both growth promoting and growth inhibitory in their effects, have been characterized including, for example, interleukins, interferons, and transforming growth factors. A number of other cytokines are known to those of skill in the art. The sources, characteristics, targets, and effector activities of these cytokines have been described.

The term “delivery vehicle” refers to any kind of device or material, which can be used to deliver cells in vivo or can be added to a composition comprising cells administered to an animal. This includes, but is not limited to, implantable devices, aggregates of cells, matrix materials, gels, etc.

As used herein, a “derivative” of a compound refers to a chemical compound that may be produced from another compound of similar structure in one or more steps, as in replacement of H by an alkyl, acyl, or amino group.

The use of the word “detect” and its grammatical variants is meant to refer to measurement of the species without quantification, whereas use of the word “determine” or “measure” with their grammatical variants are meant to refer to measurement of the species with quantification. The terms “detect” and “identify” are used interchangeably herein.

As used herein, a “detectable marker” or a “reporter molecule” is an atom or a molecule that permits the specific detection of a compound comprising the marker in the presence of similar compounds without a marker. Detectable markers or reporter molecules include, e.g., radioactive isotopes, antigenic determinants, enzymes, nucleic acids available for hybridization, chromophores, fluorophores, chemiluminescent molecules, electrochemically detectable molecules, and molecules that provide for altered fluorescence-polarization or altered light-scattering.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

As used herein, an “effective amount” means an amount sufficient to produce a selected effect. A “therapeutically effective amount” means an effective amount of an agent being used in treating or preventing a disease or disorder.

As used herein, a “functional” molecule is a molecule in a form in which it exhibits a property or activity by which it is characterized.

As used herein, a “functional biological molecule” is a biological molecule in a form in which it exhibits a property by which it is characterized. A functional enzyme, for example, is one which exhibits the characteristic catalytic activity by which the enzyme is characterized.

The term “growth factor” as used herein means a bioactive molecule that promotes the proliferation of a cell or tissue. Growth factors useful in the presently disclosed subject matter include, but are not limited to, transforming growth factor-alpha (TGF-α), transforming growth factor-beta (TGF-β), platelet-derived growth factors including the AA, AB and BB isoforms (PDGF), fibroblast growth factors (FGF), including FGF acidic isoforms 1 and 2, FGF basic form 2, and FGF 4, 8, 9, and 10, nerve growth factors (NGF) including NGF 2.5 s, NGF 7.0 s, and beta NGF and neurotrophins, brain derived neurotrophic factor, cartilage derived factor, bone growth factors (BGF), basic fibroblast growth factor, insulin-like growth factor (IGF), vascular endothelial growth factor (VEGF), EG-VEGF, VEGF-related protein, Bv8, VEGF-E, granulocyte colony stimulating factor (G-CSF), insulin like growth factor (IGF) I and II, hepatocyte growth factor, glial neurotrophic growth factor, stem cell factor (SCF), keratinocyte growth factor (KGF), skeletal growth factor, bone matrix derived growth factors, and bone derived growth factors and mixtures thereof. Some growth factors may also promote differentiation of a cell or tissue. TGF, for example, may promote growth and/or differentiation of a cell or tissue.

“Homologous” as used herein, refers to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. The homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 5′-ATTGCC-3′ and 5′-TATGGC-3′ share 50% homology.

As used herein, “homology” is used synonymously with “identity”.

The determination of percent identity between two nucleotide or amino acid sequences can be accomplished using a mathematical algorithm. For example, a mathematical algorithm useful for comparing two sequences is the algorithm of Karlin & Altschul, 1990, modified as in Karlin & Altschul, 1993). This algorithm is incorporated into the NBLAST and)(BLAST programs (see Altschul et al., 1990a; Altschul et al., 1990b), and can be accessed, for example at the National Center for Biotechnology Information (NCBI) world wide web site. BLAST nucleotide searches can be performed with the NBLAST program (designated “blastn” at the NCBI web site), using the following parameters: gap penalty=5; gap extension penalty=2; mismatch penalty=3; match reward=1; expectation value 10.0; and word size=11 to obtain nucleotide sequences homologous to a nucleic acid described herein. BLAST protein searches can be performed with the) (BLAST program (designated “blastn” at the NCBI web site) or the NCBI “blastp” program, using the following parameters: expectation value 10.0, BLOSUM62 scoring matrix to obtain amino acid sequences homologous to a protein molecule described herein. To obtain gapped alignments for comparison purposes, Gapped BLAST can be utilized as described in Altschul et al., 1997. Alternatively, PSI-Blast or PHI-Blast can be used to perform an iterated search which detects distant relationships between molecules (Id.) and relationships between molecules which share a common pattern. When utilizing BLAST, Gapped BLAST, PSI-Blast, and PHI-Blast programs, the default parameters of the respective programs (e.g., XBLAST and NBLAST) can be used.

The percent identity between two sequences can be determined using techniques similar to those described above, with or without allowing gaps. In calculating percent identity, typically exact matches are counted.

As used herein, the term “hybridization” is used in reference to the pairing of complementary nucleic acids. Hybridization and the strength of hybridization (i.e., the strength of the association between the nucleic acids) is impacted by such factors as the degree of complementarity between the nucleic acids, stringency of the conditions involved, the length of the formed hybrid, and the G:C ratio within the nucleic acids.

The term “ingredient” refers to any compound, whether of chemical or biological origin, that can be used in cell culture media to maintain or promote the proliferation, survival, or differentiation of cells. The terms “component”, “nutrient”, “supplement”, and ingredient” can be used interchangeably and are all meant to refer to such compounds. Typical non-limiting ingredients that are used in cell culture media include amino acids, salts, metals, sugars, lipids, nucleic acids, hormones, vitamins, fatty acids, proteins, and the like. Other ingredients that promote or maintain cultivation of cells ex vivo can be selected by those of skill in the art, in accordance with the particular need.

The term “inhibit”, as used herein, refers to the ability of a compound, agent, or method to reduce or impede a described function, level, activity, rate, etc., based on the context in which the term “inhibit” is used. In some embodiments, inhibition is by at least 10%, in some embodiments by at least 25%, in some embodiments by at least 50%, and in some embodiments, the function is inhibited by at least 75%. The term “inhibit” is used interchangeably with “reduce” and “block”.

The term “inhibitor” as used herein, refers to any compound or agent, the application of which results in the inhibition of a process or function of interest, including, but not limited to, differentiation and activity. Inhibition can be inferred if there is a reduction in the activity or function of interest.

As used herein “injecting or applying” includes administration of a compound of the presently disclosed subject matter by any number of routes and means including, but not limited to, topical, oral, buccal, intravenous, intramuscular, intra arterial, intramedullary, intrathecal, intraventricular, transdermal, subcutaneous, intraperitoneal, intranasal, enteral, topical, sublingual, vaginal, ophthalmic, pulmonary, or rectal means.

As used herein, “injury” generally refers to damage, harm, or hurt; usually applied to damage inflicted on the body by an external force.

As used herein, an “instructional material” includes a publication, a recording, a diagram, or any other medium of expression, which can be used to communicate the usefulness of the peptide of the presently disclosed subject matter in the kit for effecting alleviation of the various diseases or disorders recited herein. Optionally, or alternately, the instructional material may describe one or more methods of alleviating the diseases or disorders in a cell or a tissue of a mammal. The instructional material of the kit of the presently disclosed subject matter may, for example, be affixed to a container, which contains the identified compound presently disclosed subject matter, or be shipped together with a container, which contains the identified compound. Alternatively, the instructional material may be shipped separately from the container with the intention that the instructional material and the compound be used cooperatively by the recipient.

Used interchangeably herein are the terms “isolate” and “select”.

The term “isolated”, when used in reference to cells, refers to a single cell of interest, or population of cells of interest, at least partially isolated from other cell types or other cellular material with which it naturally occurs in the tissue of origin (e.g., adipose tissue). A sample of stem cells is “substantially pure” when it is in some embodiments at least 60%, in some embodiments at least 75%, in some embodiments at least 90%, and, in certain cases, in some embodiments at least 99% free of cells other than cells of interest. Purity can be measured by any appropriate method, for example, by fluorescence-activated cell sorting (FACS), or other assays, which distinguish cell types.

An “isolated nucleic acid” refers to a nucleic acid segment or fragment, which has been separated from sequences, which flank it in a naturally occurring state, e.g., a DNA fragment that has been removed from the sequences, which are normally adjacent to the fragment, e.g., the sequences adjacent to the fragment in a genome in which it naturally occurs. The term also applies to nucleic acids, which have been substantially purified, from other components, which naturally accompany the nucleic acid, e.g., RNA or DNA, or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (e.g., as a cDNA or a genomic or cDNA fragment produced by PCR or restriction enzyme digestion) independent of other sequences. It also includes a recombinant DNA, which is part of a hybrid gene encoding additional polypeptide sequence.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

As used herein, a “ligand” is a compound that specifically binds to a target compound. A ligand (e.g., an antibody) “specifically binds to” or “is specifically immunoreactive with” a compound when the ligand functions in a binding reaction which is determinative of the presence of the compound in a sample of heterogeneous compounds. Thus, under designated assay (e.g., immunoassay) conditions, the ligand binds preferentially to a particular compound and does not bind to a significant extent to other compounds present in the sample. For example, an antibody specifically binds under immunoassay conditions to an antigen bearing an epitope against which the antibody was raised. A variety of immunoassay formats may be used to select antibodies specifically immunoreactive with a particular antigen. For example, solid-phase ELISA immunoassays are routinely used to select monoclonal antibodies specifically immunoreactive with an antigen. See Harlow & Lane, 1988 for a description of immunoassay formats and conditions that can be used to determine specific immunoreactivity.

A “receptor” is a compound that specifically or selectively binds to a ligand.

As used herein, the term “linkage” refers to a connection between two groups. The connection can be either covalent or non-covalent, including but not limited to ionic bonds, hydrogen bonding, and hydrophobic/hydrophilic interactions.

As used herein, the term “linker” refers to either a molecule that joins two other molecules covalently or noncovalently, e.g., through ionic or hydrogen bonds or van der Waals interactions.

The term “measuring the level of expression” or “determining the level of expression” as used herein refers to any measure or assay which can be used to correlate the results of the assay with the level of expression of a gene or protein of interest. Such assays include measuring the level of mRNA, protein levels, etc. and can be performed by assays such as northern and western blot analyses, binding assays, immunoblots, etc. The level of expression can include rates of expression and can be measured in terms of the actual amount of an mRNA or protein present. Such assays are coupled with processes or systems to store and process information and to help quantify levels, signals, etc. and to digitize the information for use in comparing levels.

Micro-RNAs are generally about 16-25 nucleotides in length. In some embodiments, miRNAs are RNA molecules of 22 nucleotides or less in length. These molecules have been found to be highly involved in the pathology of several types of cancer. Although the miRNA molecules are generally found to be stable when associated with blood serum and its components after EDTA treatment, introduction of locked nucleic acids (LNAs) to the miRNAs via PCR further increases stability of the miRNAs. LNAs are a class of nucleic acid analogues in which the ribose ring is “locked” by a methylene bridge connecting the 2′-O atom and the 4′-C atom of the ribose ring, which increases the molecule's affinity for other molecules. miRNAs are species of small non-coding single-stranded regulatory RNAs that interact with the 3′-untranslated region (3′-UTR) of target mRNA molecules through partial sequence homology. They participate in regulatory networks as controlling elements that direct comprehensive gene expression. Bioinformatics analysis has predicted that a single miRNA can regulate hundreds of target genes, contributing to the combinational and subtle regulation of numerous genetic pathways.

The term “modulate”, as used herein, refers to changing the level of an activity, function, or process. The term “modulate” encompasses both inhibiting and stimulating an activity, function, or process. The term “modulate” is used interchangeably with the term “regulate” herein.

The term “musculoskeletal” as used herein encompasses the general broad meaning of the term, i.e., an organ system that gives a subject the ability to physically move, by using the muscles and skeletal system. Apart from locomotion, the skeleton also lends support and protects internal organs. Musculoskeletal diseases include, but are not limited to, diseases of the muscles and their associated ligaments, and other connective tissue and of the bones and cartilage viewed collectively. Musculoskeletal disorders include, for example, problems such as low back pain, joint injuries, and repetitive strain injuries of various sorts.

The term “nucleic acid” typically refers to large polynucleotides. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil).

As used herein, the term “nucleic acid” encompasses RNA as well as single and double stranded DNA and cDNA. Furthermore, the terms, “nucleic acid”, “DNA”, “RNA” and similar terms also include nucleic acid analogs, i.e. analogs having other than a phosphodiester backbone. For example, the so called “peptide nucleic acids”, which are known in the art and have peptide bonds instead of phosphodiester bonds in the backbone, are considered within the scope of the presently disclosed subject matter. By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine, and uracil). Conventional notation is used herein to describe polynucleotide sequences: the left-hand end of a single-stranded polynucleotide sequence is the 5′-end; the left-hand direction of a double-stranded polynucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand”; sequences on the DNA strand which are located 5′ to a reference point on the DNA are referred to as “upstream sequences”; sequences on the DNA strand which are 3′ to a reference point on the DNA are referred to as “downstream sequences”.

The term “nucleic acid construct”, as used herein, encompasses DNA and RNA sequences encoding the particular gene or gene fragment desired, whether obtained by genomic or synthetic methods.

Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

The term “oligonucleotide” typically refers to short polynucleotides, generally, no greater than about 50 nucleotides. It will be understood that when a nucleotide sequence is represented by a DNA sequence (i.e., A, T, G, C), this also includes an RNA sequence (i.e., A, U, G, C) in which “U” replaces “T”.

By describing two polynucleotides as “operably linked” is meant that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intraperitoneal, intramuscular, intrasternal injection, and kidney dialytic infusion techniques.

“Permeation enhancement” and “permeation enhancers” as used herein relate to the process and added materials which bring about an increase in the permeability of skin to a poorly skin permeating pharmacologically active agent, i.e., so as to increase the rate at which the drug permeates through the skin and enters the bloodstream. “Permeation enhancer” is used interchangeably with “penetration enhancer”.

The term “pharmaceutical composition” shall mean a composition comprising at least one active ingredient, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

As used herein, the term “pharmaceutically-acceptable carrier” means a chemical composition with which an appropriate compound or derivative can be combined and which, following the combination, can be used to administer the appropriate compound to a subject.

As used herein, the term “physiologically acceptable” ester or salt means an ester or salt form of the active ingredient which is compatible with any other ingredients of the pharmaceutical composition, which is not deleterious to the subject to which the composition is to be administered.

“Plurality” means at least two.

A “polynucleotide” means a single strand or parallel and anti-parallel strands of a nucleic acid. Thus, a polynucleotide may be either a single-stranded or a double-stranded nucleic acid.

“Polypeptide” refers to a polymer composed of amino acid residues, related naturally occurring structural variants, and synthetic non-naturally occurring analogs thereof linked via peptide bonds, related naturally occurring structural variants, and synthetic non-naturally occurring analogs thereof.

“Synthetic peptides or polypeptides” means a non-naturally occurring peptide or polypeptide. Synthetic peptides or polypeptides can be synthesized, for example, using an automated polypeptide synthesizer. Various solid phase peptide synthesis methods are known to those of skill in the art.

The term “prevent”, as used herein, means to stop something from happening, or taking advance measures against something possible or probable from happening. In the context of medicine, “prevention” generally refers to action taken to decrease the chance of getting a disease or condition.

“Primer” refers to a polynucleotide that is capable of specifically hybridizing to a designated polynucleotide template and providing a point of initiation for synthesis of a complementary polynucleotide. Such synthesis occurs when the polynucleotide primer is placed under conditions in which synthesis is induced, i.e., in the presence of nucleotides, a complementary polynucleotide template, and an agent for polymerization such as DNA polymerase. A primer is typically single-stranded, but may be double-stranded. Primers are typically deoxyribonucleic acids, but a wide variety of synthetic and naturally occurring primers are useful for many applications. A primer is complementary to the template to which it is designed to hybridize to serve as a site for the initiation of synthesis, but need not reflect the exact sequence of the template. In such a case, specific hybridization of the primer to the template depends on the stringency of the hybridization conditions. Primers can be labeled with, e.g., chromogenic, radioactive, or fluorescent moieties and used as detectable moieties.

A “prophylactic” treatment is a treatment administered to a subject who does not exhibit signs of a disease or injury or exhibits only early signs of the disease or injury for the purpose of decreasing the risk of developing pathology associated with the disease or injury.

As used herein, the term “promoter/regulatory sequence” means a nucleic acid sequence which is required for expression of a gene product operably linked to the promoter/regulator sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a promoter which drives expression of a gene to which it is operably linked, in a constant manner in a cell. By way of example, promoters which drive expression of cellular housekeeping genes are considered to be constitutive promoters.

An “inducible” promoter is a nucleotide sequence which, when operably linked with a polynucleotide which encodes or specifies a gene product, causes the gene product to be produced in a living cell substantially only when an inducer which corresponds to the promoter is present in the cell.

A “tissue-specific” promoter is a nucleotide sequence which, when operably linked with a polynucleotide which encodes or specifies a gene product, causes the gene product to be produced in a living cell substantially only if the cell is a cell of the tissue type corresponding to the promoter.

As used herein, “protecting group” with respect to a terminal amino group refers to a terminal amino group of a peptide, which terminal amino group is coupled with any of various amino-terminal protecting groups traditionally employed in peptide synthesis. Such protecting groups include, for example, acyl protecting groups such as formyl, acetyl, benzoyl, trifluoroacetyl, succinyl, and methoxysuccinyl; aromatic urethane protecting groups such as benzyloxycarbonyl; and aliphatic urethane protecting groups, for example, tert-butoxycarbonyl or adamantyloxycarbonyl. See Gross & Mienhofer, 1981 for suitable protecting groups.

As used herein, “protecting group” with respect to a terminal carboxy group refers to a terminal carboxyl group of a peptide, which terminal carboxyl group is coupled with any of various carboxyl-terminal protecting groups. Such protecting groups include, for example, tert-butyl, benzyl, or other acceptable groups linked to the terminal carboxyl group through an ester or ether bond.

The term “protein” typically refers to large polypeptides. Conventional notation is used herein to portray polypeptide sequences: the left-hand end of a polypeptide sequence is the amino-terminus; the right-hand end of a polypeptide sequence is the carboxyl-terminus.

The term “protein regulatory pathway”, as used herein, refers to both the upstream regulatory pathway which regulates a protein, as well as the downstream events which that protein regulates. Such regulation includes, but is not limited to, transcription, translation, levels, activity, posttranslational modification, and function of the protein of interest, as well as the downstream events which the protein regulates.

The terms “protein pathway” and “protein regulatory pathway” are used interchangeably herein.

As used herein, the term “purified” and like terms relate to an enrichment of a molecule or compound relative to other components normally associated with the molecule or compound in a native environment. The term “purified” does not necessarily indicate that complete purity of the particular molecule has been achieved during the process. A “highly purified” compound as used herein refers to a compound that is greater than 90% pure.

“Recombinant polynucleotide” refers to a polynucleotide having sequences that are not naturally joined together. An amplified or assembled recombinant polynucleotide may be included in a suitable vector, and the vector can be used to transform a suitable host cell.

A recombinant polynucleotide may serve a non-coding function (e.g., promoter, origin of replication, ribosome-binding site, etc.) as well.

A host cell that comprises a recombinant polynucleotide is referred to as a “recombinant host cell”. A gene which is expressed in a recombinant host cell wherein the gene comprises a recombinant polynucleotide, produces a “recombinant polypeptide”.

A “recombinant polypeptide” is one which is produced upon expression of a recombinant polynucleotide.

The term “regulate” refers to either stimulating or inhibiting a function or activity of interest.

As used herein, term “regulatory elements” is used interchangeably with “regulatory sequences” and refers to promoters, enhancers, and other expression control elements, or any combination of such elements.

A “reversibly implantable” device is one which may be inserted (e.g., surgically or by insertion into a natural orifice of the animal) into the body of an animal and thereafter removed without great harm to the health of the animal.

A “sample”, as used herein, refers in some embodiments to a biological sample from a subject, including, but not limited to, normal tissue samples, diseased tissue samples, biopsies, blood, saliva, feces, semen, tears, and urine. A sample can also be any other source of material obtained from a subject which contains cells, tissues, or fluid of interest. A sample can also be obtained from cell or tissue culture.

A “significant detectable level” is an amount of contaminate that would be visible in the presented data and would need to be addressed/explained during analysis of the forensic evidence.

By the term “signal sequence” is meant a polynucleotide sequence which encodes a peptide that directs the path a polypeptide takes within a cell, i.e., it directs the cellular processing of a polypeptide in a cell, including, but not limited to, eventual secretion of a polypeptide from a cell. A signal sequence is a sequence of amino acids which are typically, but not exclusively, found at the amino terminus of a polypeptide which targets the synthesis of the polypeptide to the endoplasmic reticulum. In some instances, the signal peptide is proteolytically removed from the polypeptide and is thus absent from the mature protein.

By “small interfering RNAs (siRNAs)” is meant, inter alia, an isolated dsRNA molecule comprised of both a sense and an anti-sense strand. In some embodiments, it is greater than 10 nucleotides in length. siRNA also refers to a single transcript which has both the sense and complementary antisense sequences from the target gene, e.g., a hairpin. siRNA further includes any form of dsRNA (proteolytically cleaved products of larger dsRNA, partially purified RNA, essentially pure RNA, synthetic RNA, recombinantly produced RNA) as well as altered RNA that differs from naturally occurring RNA by the addition, deletion, substitution, and/or alteration of one or more nucleotides.

The terms “solid support”, “surface” and “substrate” are used interchangeably and refer to a structural unit of any size, where said structural unit or substrate has a surface suitable for immobilization of molecular structure or modification of said structure and said substrate is made of a material such as, but not limited to, metal, metal films, glass, fused silica, synthetic polymers, and membranes.

By the term “specifically binds”, as used herein, is meant a molecule which recognizes and binds a specific molecule, but does not substantially recognize or bind other molecules in a sample, or it means binding between two or more molecules as in part of a cellular regulatory process, where said molecules do not substantially recognize or bind other molecules in a sample.

The term “standard”, as used herein, refers to something used for comparison. For example, it can be a known standard agent or compound which is administered and used for comparing results when administering a test compound, or it can be a standard parameter or function which is measured to obtain a control value when measuring an effect of an agent or compound on a parameter or function. “Standard” can also refer to an “internal standard”, such as an agent or compound which is added at known amounts to a sample and which is useful in determining such things as purification or recovery rates when a sample is processed or subjected to purification or extraction procedures before a marker of interest is measured. Internal standards are often but are not limited to, a purified marker of interest which has been labeled, such as with a radioactive isotope, allowing it to be distinguished from an endogenous substance in a sample.

The term “stimulate” as used herein, means to induce or increase an activity or function level such that it is higher relative to a control value. The stimulation can be via direct or indirect mechanisms. In some embodiments, the activity or function is stimulated by at least 10% compared to a control value, in some embodiments by at least 25%, and in some embodiments by at least 50%. The term “stimulator” as used herein, refers to any composition, compound or agent, the application of which results in the stimulation of a process or function of interest, including, but not limited to, wound healing, angiogenesis, bone healing, osteoblast production and function, and osteoclast production, differentiation, and activity.

A “subject” of diagnosis or treatment is an animal, including a human. It also includes pets and livestock.

As used herein, a “subject in need thereof” is a patient, animal, mammal, or human, who will benefit from the method of the presently disclosed subject matter.

As used herein, “substantially homologous amino acid sequences” includes those amino acid sequences which have at least about 95% homology, in some embodiments at least about 96% homology, more in some embodiments at least about 97% homology, in some embodiments at least about 98% homology, and most in some embodiments at least about 99% or more homology to an amino acid sequence of a reference sequence. Amino acid sequence similarity or identity can be computed by using the BLASTP and TBLASTN programs which employ the BLAST (basic local alignment search tool) 2.0.14 algorithm. The default settings used for these programs are suitable for identifying substantially similar amino acid sequences for purposes of the presently disclosed subject matter.

“Substantially homologous nucleic acid sequence” means a nucleic acid sequence corresponding to a reference nucleic acid sequence wherein the corresponding sequence encodes a peptide having substantially the same structure and function as the peptide encoded by the reference nucleic acid sequence; e.g., where only changes in amino acids not significantly affecting the peptide function occur. In some embodiments, the substantially identical nucleic acid sequence encodes the peptide encoded by the reference nucleic acid sequence. The percentage of identity between the substantially similar nucleic acid sequence and the reference nucleic acid sequence is at least about 50%, 65%, 75%, 85%, 95%, 99% or more. Substantial identity of nucleic acid sequences can be determined by comparing the sequence identity of two sequences, for example by physical/chemical methods (i.e., hybridization) or by sequence alignment via computer algorithm. Suitable nucleic acid hybridization conditions to determine if a nucleotide sequence is substantially similar to a reference nucleotide sequence are: 7% sodium dodecyl sulfate SDS, 0.5 M NaPO4, 1 mM EDTA at 50° C. with washing in 2× standard saline citrate (SSC), 0.1% SDS at 50° C.; in some embodiments in 7% (SDS), 0.5 M NaPO4, 1 mM EDTA at 50° C. with washing in 1×SSC, 0.1% SDS at 50° C.; in some embodiments 7% SDS, 0.5 M NaPO4, 1 mM EDTA at 50° C. with washing in 0.5×SSC, 0.1% SDS at 50° C.; and more in some embodiments in 7% SDS, 0.5 M NaPO4, 1 mM EDTA at 50° C. with washing in 0.1×SSC, 0.1% SDS at 65° C. Suitable computer algorithms to determine substantial similarity between two nucleic acid sequences include, GCS program package (Devereux et al., 1984), and the BLASTN or FASTA programs (Altschul et al., 1990a; Altschul et al., 1990b; Altschul et al., 1997). The default settings provided with these programs are suitable for determining substantial similarity of nucleic acid sequences for purposes of the presently disclosed subject matter.

The term “substantially pure” describes a compound, e.g., a protein or polypeptide which has been separated from components which naturally accompany it. Typically, a compound is substantially pure when at least 10%, more in some embodiments at least 20%, more in some embodiments at least 50%, more in some embodiments at least 60%, more in some embodiments at least 75%, more in some embodiments at least 90%, and most in some embodiments at least 99% of the total material (by volume, by wet or dry weight, or by mole percent or mole fraction) in a sample is the compound of interest. Purity can be measured by any appropriate method, e.g., in the case of polypeptides by column chromatography, gel electrophoresis, or HPLC analysis. A compound, e.g., a protein, is also substantially purified when it is essentially free of naturally associated components or when it is separated from the native contaminants which accompany it in its natural state.

A “surface active agent” or “surfactant” is a substance that has the ability to reduce the surface tension of materials and enable penetration into and through materials.

The term “symptom”, as used herein, refers to any morbid phenomenon or departure from the normal in structure, function, or sensation, experienced by the patient and indicative of disease. In contrast, a “sign” is objective evidence of disease. For example, a bloody nose is a sign. It is evident to the patient, doctor, nurse, and other observers.

A “therapeutic” treatment is a treatment administered to a subject who exhibits signs of pathology for the purpose of diminishing or eliminating those signs.

A “therapeutically effective amount” of a compound is that amount of compound which is sufficient to provide a beneficial effect to the subject to which the compound is administered.

“Tissue” means (1) a group of similar cell united perform a specific function; (2) a part of an organism consisting of an aggregate of cells having a similar structure and function; or (3) a grouping of cells that are similarly characterized by their structure and function, such as muscle or nerve tissue.

The term “tissue injury-associated decreased blood flow”, as used herein, refers to the decrease in blood flow which occurs following an injury, such as a wound, a fracture, a surgical procedure, or a thermal injury. The decrease in blood flow includes, but is not limited to, decreased volume, rate, stasis, or sludging. One of ordinary skill in the art will appreciate that there are multiple parameters which can be used as measures or signs of decreased blood flow, as well as multiple techniques to determine decreased blood flow.

The term “topical application”, as used herein, refers to administration to a surface, such as the skin. This term is used interchangeably with “cutaneous application” in the case of skin. A “topical application” is a “direct application”.

By “transdermal” delivery is meant delivery by passage of a drug through the skin or mucosal tissue and into the bloodstream. Transdermal also refers to the skin as a portal for the administration of drugs or compounds by topical application of the drug or compound thereto. “Transdermal” is used interchangeably with “percutaneous”.

The term “transfection” is used interchangeably with the terms “gene transfer”, “transformation”, and “transduction”, and means the intracellular introduction of a polynucleotide. “Transfection efficiency” refers to the relative amount of the transgene taken up by the cells subjected to transfection. In practice, transfection efficiency is estimated by the amount of the reporter gene product expressed following the transfection procedure.

As used herein, the term “transgene” means an exogenous nucleic acid sequence comprising a nucleic acid which encodes a promoter/regulatory sequence operably linked to nucleic acid which encodes an amino acid sequence, which exogenous nucleic acid is encoded by a transgenic mammal.

As used herein, the term “treating” may include prophylaxis of the specific injury, disease, disorder, or condition, or alleviation of the symptoms associated with a specific injury, disease, disorder, or condition and/or preventing or eliminating said symptoms. A “prophylactic” treatment is a treatment administered to a subject who does not exhibit signs of a disease or exhibits only early signs of the disease for the purpose of decreasing the risk of developing pathology associated with the disease. “Treating” is used interchangeably with “treatment” herein.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer or delivery of nucleic acid to cells, such as, for example, polylysine compounds, liposomes, and the like. Examples of viral vectors include, but are not limited to, adenoviral vectors, adeno-associated virus vectors, retroviral vectors, recombinant viral vectors, and the like. Examples of non-viral vectors include, but are not limited to, liposomes, polyamine derivatives of DNA and the like.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses that incorporate the recombinant polynucleotide.

As used herein “wound” or “wounds” may refer to any detectable break in the tissues of the body, such as injury to skin or to an injury or damage, or to a damaged site associated with a disease or disorder. As used herein, the term “wound” relates to a physical tear, break, or rupture to a tissue or cell layer. A wound may occur by any physical insult, including a surgical procedure or as a result of a disease, disorder condition. Although the terms “wound” and “injury” are not always defined exactly the same way, the use of one term herein, such as “injury”, is not meant to exclude the meaning of the other term.

### II.B. Chemical Definitions

As used herein, the term “halogen” or “halo” includes bromo, chloro, fluoro, and iodo.

The term “haloalkyl” as used herein refers to an alkyl radical bearing at least one halogen substituent, for example, chloromethyl, fluoroethyl or trifluoromethyl and the like.

The term “C₁-Cₙ alkyl” wherein n is an integer, as used herein, represents a branched or linear alkyl group having from one to the specified number of carbon atoms. Typically, C₁-C₆ alkyl groups include, but are not limited to, methyl, ethyl, n-propyl, iso-propyl, butyl, iso-butyl, sec-butyl, tert-butyl, pentyl, hexyl, and the like.

The term “C₂-Cₙ alkenyl” wherein n is an integer, as used herein, represents an olefinically unsaturated branched or linear group having from two to the specified number of carbon atoms and at least one double bond. Examples of such groups include, but are not limited to, 1-propenyl, 2-propenyl, 1,3-butadienyl, 1-butenyl, hexenyl, pentenyl, and the like.

The term “C₂-Cₙ alkynyl” wherein n is an integer refers to an unsaturated branched or linear group having from two to the specified number of carbon atoms and at least one triple bond. Examples of such groups include, but are not limited to, 1-propynyl, 2-propynyl, 1-butynyl, 2-butynyl, 1-pentynyl, and the like.

The term “C₃-Cₙ cycloalkyl” wherein n=8, represents cyclopropyl, cyclobutyl, cyclopentyl, cyclohexyl, cycloheptyl, and cyclooctyl.

As used herein the term “aryl” refers to an optionally substituted mono- or bicyclic carbocyclic ring system having one or two aromatic rings including, but not limited to, phenyl, benzyl, naphthyl, tetrahydronaphthyl, indanyl, indenyl, and the like. AOptionally substituted aryl @ includes aryl compounds having from zero to four substituents, and Asubstituted aryl @ includes aryl compounds having one or more substituents. The term (C₅-C₈ alkyl)aryl refers to any aryl group which is attached to the parent moiety via the alkyl group.

The term “bicyclic” represents either an unsaturated or saturated stable 7- to 12-membered bridged or fused bicyclic carbon ring. The bicyclic ring may be attached at any carbon atom which affords a stable structure. The term includes, but is not limited to, naphthyl, dicyclohexyl, dicyclohexenyl, and the like.

The term “heterocyclic group” refers to an optionally substituted mono- or bicyclic carbocyclic ring system containing from one to three heteroatoms wherein the heteroatoms are selected from the group consisting of oxygen, sulfur, and nitrogen.

As used herein the term “heteroaryl” refers to an optionally substituted mono- or bicyclic carbocyclic ring system having one or two aromatic rings containing from one to three heteroatoms and includes, but is not limited to, furyl, thienyl, pyridyl and the like.

As used herein, the term “optionally substituted” refers to from zero to four substituents, wherein the substituents are each independently selected. Each of the independently selected substituents may be the same or different than other substituents.

The compounds of the presently disclosed subject matter contain one or more asymmetric centers in the molecule. In accordance with the presently disclosed subject matter a structure that does not designate the stereochemistry is to be understood as embracing all the various optical isomers, as well as racemic mixtures thereof.

The compounds of the presently disclosed subject matter may exist in tautomeric forms and the presently disclosed subject matter includes both mixtures and separate individual tautomers. For example the following structure:

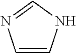

is understood to represent a mixture of the structures:

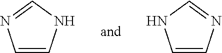 [SMILES: c1c[nH]cn1 ; c1c[nH]cn1]

The terminology used herein is for the purpose of describing the particular versions or embodiments only, and is not intended to limit the scope of the presently disclosed subject matter. All publications mentioned herein are incorporated by reference in their entirety.

### III. Embodiments

### III.A. Generally

It is disclosed herein that S1R is a novel inhibitor of inflammation in a model of bacterial LPS-induced septic shock. Mice lacking S1R are highly susceptible to an otherwise non-lethal dose of LPS in vivo, with concentrations of circulating inflammatory cytokines rising to critical levels shortly after LPS administration. S1R deficient bone marrow derived macrophages also exhibit increased LPS-induced production of pro-inflammatory cytokines. Mechanistically, it is disclosed that S1R controls ER mediated inflammation by specific regulation of the IRE1 endonuclease activity. Finally, administration of an IRE1 specific endonuclease inhibitor was able to protect S1R deficient mice from death in a model of septic shock, confirming in vivo the relevance of the S1R-IRE1 interaction during LPS exposure.

Without wishing to be bound by any particular theory, it is hypothesized that the presently disclosed subject matter has uncovered a novel contribution of sigma-1 during inflammatory responses. In some embodiments, the modulation of Sigma-1 receptor activity can be used to treat conditions when the inflammatory response is detrimental or impaired.

In some embodiments, a method for treating inflammation in a subject in need thereof is provided in accordance with the presently disclosed subject matter. In some embodiments, the method comprising administering to the subject an effective amount of a Sigma-1 receptor (S1R) activity modulator to thereby treat inflammation in the subject. In some embodiments, modulation of S1R activity treats conditions where an inflammatory response in a subject is detrimental or impaired. In some embodiments, the inflammation is associated with septic shock.

In some embodiments, the presently disclosed subject matter provides a pharmaceutical composition comprising, consisting essentially of, or consisting of an effective amount of a Sigma-1 receptor (S1R) activity modulator to treat inflammation in a subject in need thereof. In some embodiments, the presently disclosed subject matter provide the use of an effective amount of a Sigma-1 receptor (S1R) activity modulator for the preparation of a medicament to treat inflammation in a subject in need thereof. In some embodiments, the presently disclosed subject matter, the presently disclosed subject matter provides a pharmaceutical composition comprising, consisting essentially of, or consisting of an effective amount of a Sigma-1 receptor (S1R) activity modulator to treat inflammation in a subject in need thereof.

The term “modulate”, as used herein, refers to changing the level of an activity, function, or process. The term “modulate” encompasses both agonizing and antagonizing, and/or inhibiting and stimulating an activity, function, or process. The term “modulate” is used interchangeably with the term “regulate” herein. By “modulate” is intended an increase, decrease, or other alteration of any or all biological activities or properties of S1R. A composition that has an ability to modulate S1R biological activity has utility in the treatment of disorders and conditions associated with the biological activity of S1R, including modulating IRE1 biological activity and/or modulating inflammation.

In some embodiments, the S1R activity modulator is a S1R agonist. In some embodiments, the S1R agonist is selected from the group consisting of wherein is a S1R agonist is selected from the group consisting of PRS-013, SA-4503, siramesine, (+)-pentazocine, (+)-SKF10,047, PRE084 (2-morpholin-4-ylethyl 1-phenylcyclohexane-1-carboxylate), SA4503 (1-[2-(3,4-dimethoxyphenyl)ethyl]-4-(3-phenylpropyl)piperazine), (±)-PPCC oxalate, PRE-084 hydrochloride, SA 4503 dihydrochloride, (+)-SK&F 10047 hydrochloride, and a compound commercially available under the following trade names ANAVEX® 2-73, ANAVEX® 3-71, ANAVEX® 1-51, ANAVEX® 1079, ANAVEX® 1067, ANAVEX® 1037, ANAVEX® 1519, and ANAVEX® 1066.

In some embodiments, the S1R activity modulator is a composition that increases a level of S1R in the subject. In some embodiments, the composition that increases a level of S1R in the subject comprises an expression vector that expresses S1R.

In some embodiments, the S1R activity modulator comprises an oligonucleotide.

In some embodiments, two or more S1R activity modulators are administered in combination. In some embodiments, an additional therapeutic agent is administered. In some embodiments, the additional therapeutic agent is an IRE1 specific endonuclease inhibitor. In some embodiments, the IRE1 specific endonuclease inhibitor is selected from the group consisting of 4μ8C (7-Hydroxy-4-methyl-2-oxo-2H-1-benzopyran-8-carboxaldehyde), STF 083010 (N-[(2-Hydroxy-1-naphthalenyl)methylene]-2-thiophenesulfonamide), MKC8866 (CAS #1338934-59-0), Kira 6 (CAS #1589527-65-0), Kira 8 (CAS #1630086-20-2), MKC3946 (CAS #1093119-54-0), GSK2850163 (CAS #2121989-91-9), 6-Bromo-2-hydroxy-3-methoxybenzaldehyde (CAS #20035-41-0), 3-methoxy-6-bromosalicylaldehyde salicylaldimines, toyocamycin, N⁹-(3-(dimethylamino) propyl)-N³,N³,N⁶,N⁶-tetramethylacridine-3,6,9-triamine (3,6-DMAD), Hydroxy-aryl-aldehydes (HAA), and irestatin.

Thus, it is disclosed herein that S1R is a novel inhibitor of inflammation. In one aspect, methods of the presently disclosed subject matter are useful for preventing and treating septic shock. It is disclosed herein that S1R controls ER mediated inflammation by specific regulation of the IRE1 endonuclease activity. It is disclosed herein that administration of an IRE1 specific endonuclease inhibitor protects S1R deficient mice from death in a model of septic shock, confirming in vivo the relevance of the S1R-IRE1 interaction during LPS exposure. Further support for the role of S1R in inflammation is the present disclosure that lack of S1R increases susceptibility to septic shock. It is also disclosed herein that S1R-deficient macrophages have increased production of inflammatory cytokines. The disclosed results further suggest that, while S1R acts a robust inhibitor of inflammation, it does not regulate the inflammatory response to LPS via the canonical inflammatory signaling pathways: NF-κB, ERK1/2, and JNK.

The presently disclosed subject matter provides that overexpression of S1R results in a significant decrease in IRE1 abundance relative to the empty vector control and that inhibition of IRE1 endonuclease activity rescues S1R KO mice from septic mortality. The presently disclosed subject matter provides compositions and methods useful for regulating LPS-induced cytokine production.

The presently disclosed subject matter provides compositions and method useful for inhibiting inflammation. In one aspect, the inflammation is associated with septic shock.

The presently disclosed subject matter further provides for regulation of S1R to regulate inflammation. Compositions and methods are provided to stimulate S1R activity or to increase S1R activity or levels. In one aspect, compositions and methods are provided to stimulate S1R expression. In another aspect, compositions and methods are provided to increase S1R levels. In one aspect, stimulating S1R activity, levels, or expression inhibits IRE1 activity or levels.

S1R activity, levels, and expression can be increased or stimulated using various kinds of S1R activity modulator compounds, including but not limited to drugs, expression vectors comprising nucleic acids encoding S1R, oligonucleotides, S1R protein, and upstream stimulators of S1R. In some embodiments, the S1R activity modulator comprises an oligonucleotide. Exemplary oligonucleotides that can be employed as S1R activity modulators include, but are not limited to miRNAs, which in some embodiments can interact with S1R gene products (e.g., S1R mRNAs) to stabilize the S1R gene products, thereby permitting increased expression of the S1R polypeptides. By way of example and not limitation, the human S1R gene product represented by Accession No. NM 005866.4 of the GENBANK® biosequence database (SEQ ID NO: 1) includes at least two highly conserved miRNA sequences that can be employed for increasing expression of human S1R gene products and/or polypeptides. These miRNA sequences include hsa-miR-153-3p (5′-uugcauagucacaaaagugauc-3′; SEQ ID NO: 5) and hsa-miR-124-3p (5′-cguguucacagcggaccuugau-3′; SEQ ID NO: 6), although other miRNAs that are predicted to interact with human S1R gene products include hsa-miR-574-5p (5′-ugagugugugugugugagugugu-3′; SEQ ID NO: 7), hsa-miR-8485 (5′-cacacacacacacacacguau-3′; SEQ ID NO: 8), and hsa-miR-6724-5p (5′-cugggcccgcggcgggcgugggg-3′; SEQ ID NO: 9). See the miRbase website managed by the Griffiths-Jones lab at the Faculty of Biology, Medicine and Health, University of Manchester, Manchester, United Kingdom. See also Griffiths-Jones, 2004; Griffiths-Jones et al., 2006; Griffiths-Jones et al., 2008; Kozomara et al., 2011; Kozomara et al., 2014; and Kozomara et al., 2019; each of which is incorporated by reference in its entirety.

The presently disclosed subject matter provides for the use of pharmaceutical compositions comprising an effective amount of a useful drug or compound with the activity disclosed herein. In one aspect, the compound is an agonist of S1R activity. Useful compounds include, for example, tetrahydro-N,N-dimethyl-2,2-diphenyl-3-furanmethanamine hydrochloride, and tetrahydro-N,N-dimethyl-5,5-diphenyl-3-furanmethanamine hydrochloride.

Useful compounds of the presently disclosed subject matter for regulating S1R include, but are not limited to, ANAVEX® 2-73, ANAVEX® 3-71, ANAVEX® 1-51, ANAVEX® 1079, ANAVEX® 1067, ANAVEX® 1037, ANAVEX® 1519, and ANAVEX® 1066.

ANAVEX® 2-73 (tetrahydro-N,N-dimethyl-2,2-diphenyl-3-furanmethanamine hydrochloride), has been indicated for treatment of conditions such as Alzheimer's Disease. It is an agonist of the intracellular sigma chaperone protein and is a mixed ligand for sigma1/muscarinic receptors. it is expressed in most tissues and located at focal contacts between mitochondria and the endoplasmic reticulum. The sigma-1 receptor forms heterodimers with many other membrane receptors, and influences multiple cellular pathways and physiological processes. it appears that ANAVEX® 2-73 binds the sigma-1 receptor in the high nanomolar range and the muscarinic receptor in the low micromolar range. It has been reported to have memory-preserving and neuroprotective effects in mice treated with the muscarinic receptor antagonist scopolamine, with synthetic Aβ oligomer injection, or with the NMDA receptor agonist dizocilpine. A recent study suggested that ANAVEX® 2-73 may block tau hyperphosphorylation.

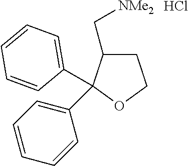 [SMILES: CN(C)CC1CCOC1(c1ccccc1)c1ccccc1 ; Cl]

ANAVEX® 3-71 (1-(2,8-dimethyl-1-thia-3,8-diazaspiro(4.5)dec-3-yl)-3-(1H-indol-3-yl)propan-1-one), previously named AF710B, elicits its effects using a distinct mechanism of action via sigma-1 receptor activation and M1 muscarinic allosteric modulation, which has shown to enhance neuroprotection and cognition in Alzheimer's disease. ANAVEX® 3-71 is a CNS-penetrable mono-therapy that bridges treatment of both cognitive impairments with disease modifications. It is highly effective in very small doses against the major Alzheimer's hallmarks in transgenic (3×Tg-AD) mice, including cognitive deficits, amyloid and tau pathologies, and also has beneficial effects on inflammation and mitochondrial dysfunctions.

ANAVEX® 1-41 (tetrahydro-N,N-dimethyl-5,5-di phenyl-3-furanmethanamine hydrochloride) is a sigma-1 agonist. Pre-clinical tests revealed significant neuroprotective benefits through the modulation of endoplasmic reticulum, mitochondrial and oxidative stress. It has also been shown to prevent expression of caspase-3, an enzyme that plays a key role in apoptosis and loss of cells in the hippocampus, the part of the brain that regulates learning, emotion, and memory.

ANAVEX® 1037 is a low molecular weight, synthetic compound exhibiting high affinity for sigma-1 receptors at nanomolar levels and moderate affinity for sigma-2 receptors and sodium channels at micromolar levels. In advanced preclinical studies, this compound had no toxic side effects.

ANAVEX® PLUS is a combination of ANAVEX® 2-73 and donepezil (ARICEPT®).

The presently disclosed subject matter encompasses the use of one or more of the drugs described herein to regulate S1R as well as additional therapeutic agents. In one aspect, two or more drugs regulating inflammation are used in combination therapy.

The presently disclosed subject matter further provides compositions and methods for regulating inflammation by inhibiting IRE1 activity or levels. In one aspect, IRE1 is inhibited directly. In one aspect, the inhibitor blocks an upstream stimulator of IRE1. In one aspect, the inhibitor of IRE1 activity or levels stimulates and upstream inhibitor of IRE1 activity or levels. In one aspect, an inhibitor of IRE1 is selected from the group consisting of 4μ8C (7-Hydroxy-4-methyl-2-oxo-2H-1-benzopyran-8-carboxaldehyde), STF 083010 (N-[(2-Hydroxy-1-naphthalenyl)methylene]-2-thiophenesulfonamide), MKC8866 (CAS #1338934-59-0), Kira 6 (CAS #1589527-65-0), Kira 8 (CAS #1630086-20-2), MKC3946 (CAS #1093119-54-0), GSK2850163 (CAS #2121989-91-9), 6-Bromo-2-hydroxy-3-methoxybenzaldehyde (CAS #20035-41-0), 3-methoxy-6-bromosalicylaldehyde salicylaldimines, toyocamycin, N⁹-(3-(dimethylamino) propyl)-N³,N³,N⁶,N⁶-tetramethylacridine-3,6,9-triamine (3,6-DMAD), Hydroxy-aryl-aldehydes (HAA), and irestatin.

Compounds binding to the sigma receptor as defined herein, may be antagonists, inverse agonists, agonists, partial antagonists and/or partial agonists.

Methods and concentrations for dosing with compounds such as ANAVEX® 2-73 are provided herein or are known in the art (although for other uses), for example, see U.S. Pat. No. 9,750,746, incorporated herein by reference in its entirety. See also U.S. Patent Application Publication Nos. 2015/0073046, 2016/0009674, and 2017/0360798, each of which is incorporated herein by reference in its entirety.

Examples of other potentially useful compounds, include, but are not limited to, ANAVEX-27-1041, PRS-013, SA-4503, siramesine, ANAVEX-7-1037, (+)-pentazocine, (+)-SKF 10,047, PRE084 (2-morpholin-4-ylethyl 1-phenylcyclohexane-1-carboxylate), SA4503 (1-[2-(3,4-dimethoxyphenyl)ethyl]-4-(3-phenylpropyl)piperazine), (±)-PPCC oxalate, PRE-084 hydrochloride, SA 4503 dihydrochloride, and (+)-SK&F 10047 hydrochloride.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

The compounds of the presently disclosed subject matter may be administered to, for example, a cell, a tissue, or a subject by any of several methods described herein and by others which are known to those of skill in the art.

The relative amounts of the active ingredient, the pharmaceutically acceptable carrier, and any additional ingredients in a pharmaceutical composition of the presently disclosed subject matter will vary, depending upon the identity, sex, age, size, and condition of the subject treated and further depending upon the route by which the composition is to be administered.

In addition to the active ingredient, a composition of the presently disclosed subject matter may further comprise one or more additional pharmaceutically active or therapeutic agents. Particularly contemplated additional agents include anti-emetics and scavengers such as cyanide and cyanate scavengers.

Controlled- or sustained-release formulations of a composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Gennaro, 1990 and/or Gennaro, 2003, each of which is incorporated herein by reference.

Other components such as preservatives, antioxidants, surfactants, absorption enhancers, viscosity enhancers or film forming polymers, bulking agents, diluents, coloring agents, flavoring agents, pH modifiers, sweeteners or taste-masking agents may also be incorporated into the composition. Suitable coloring agents include red, black, and yellow iron oxides and FD&C dyes such as FD&C Blue No. 2, FD&C Red No. 40, and the like. Suitable flavoring agents include mint, raspberry, licorice, orange, lemon, grapefruit, caramel, vanilla, cherry grape flavors, combinations thereof, and the like. Suitable pH modifiers include citric acid, tartaric acid, phosphoric acid, hydrochloric acid, maleic acid, sodium hydroxide, and the like. Suitable sweeteners include aspartame, acesulfame K, thaumatic, and the like. Suitable taste-masking agents include sodium bicarbonate, ion-exchange resins, cyclodextrin inclusion compounds, adsorbates, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, and birds including commercially relevant birds such as chickens, ducks, geese, and turkeys.

The pharmaceutical compositions of the presently disclosed subject matter can be administered in any suitable formulation, by any suitable means, and by any suitable route of administration. Formulations suitable for topical administration include, but are not limited to, liquid or semi-liquid preparations such as liniments, lotions, oil in water or water in oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

In accordance with the presently disclosed subject matter, as described above or as discussed in the Examples below, there can be employed conventional chemical, cellular, histochemical, biochemical, molecular biology, microbiology, recombinant DNA, and clinical techniques which are known to those of skill in the art. Such techniques are explained fully in the literature. See for example, Sambrook et al., 1989; Glover, 1985; Gait, 1984; Harlow & Lane, 1988; Roe et al., 1996; and Ausubel et al., 1995.

The presently disclosed subject matter may be embodied in other specific forms without departing from the spirit or essential attributes thereof. The presently disclosed subject matter encompasses all combinations of the different aspects of the presently disclosed subject matter noted herein. It is understood that any and all embodiments of the presently disclosed subject matter may be taken in conjunction with any other embodiment or embodiments to describe additional more preferred embodiments. It is also to be understood that each individual element of the preferred embodiments is intended to be taken individually as its own independent preferred embodiment. Furthermore, any element of an embodiment is meant to be combined with any and all other elements from any embodiment to describe an additional embodiment.

Typically, dosages of the compounds of the presently disclosed subject matter which may be administered to an animal, in some embodiments a human, range in amount from about 1.0 μg to about 100 g per kilogram of body weight of the animal. The precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In some embodiments, the dosage will vary from about 10 mg to about 1 g per kilogram of body weight of the animal.

The compounds may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

### III.B. Gene Therapy Formats and Preparation Thereof

Gene therapy constructs against proteins, polypeptides, or peptide fragments thereof of the presently disclosed subject matter may be generated using methods that are well known in the art. By way of example and not limitation, S1R genes can be used for expression of S1R in accordance with the presently disclosed subject matter. Exemplary methods are described in U.S. Patent Application Publication Nos. 2019/0000991 and 2019/0008909, each of which is herein incorporated by reference in its entirety.

Briefly, vector therapy directed toward modulation of S1R levels, to thereby affect or modulate the biological activity of IRE1 in a target cell or tissue is described. In some embodiments, a therapeutic method of the presently disclosed subject matter a process for modulation of S1R levels comprising the steps of: (a) delivering to the cell an effective amount of a DNA molecule comprising a polynucleotide that encodes a polypeptide that modulates the biological activity of S1R; and (b) maintaining the cell under conditions sufficient for expression of said polypeptide.

In accordance with the presently disclosed subject matter a S1R gene sequence itself is employed to introduce a S1R gene product, a convenient method of introduction will be through the use of a recombinant vector that incorporates the desired gene, together with its associated control sequences. The preparation of recombinant vectors is well known to those of skill in the art and described in many references, such as, for example, Green et al., 2014, incorporated herein in its entirety.

It is understood that the DNA coding sequences to be expressed, in this case those encoding the S1R gene products, are positioned in a vector adjacent to and under the control of a promoter. It is understood in the art that to bring a coding sequence under the control of such a promoter, one generally positions the 5′ end of the transcription initiation site of the transcriptional reading frame of the gene product to be expressed between about 1 and about 50 nucleotides “downstream” of (i.e., 3′ of) the chosen promoter.

Thus, a promoter is a region of a DNA molecule typically within about 100 nucleotide pairs upstream of (i.e., 5′ to) the point at which transcription begins (i.e., a transcription start site). That region typically contains several types of DNA sequence elements that are located in similar relative positions in different genes. Another type of discrete transcription regulatory sequence element is an enhancer. An enhancer imposes specificity of time, location and expression level on a particular coding region or gene. A major function of an enhancer is to increase the level of transcription of a coding sequence in a cell that contains one or more transcription factors that bind to that enhancer. An enhancer can function when located at variable distances from transcription start sites so long as a promoter is present.

As used herein, the phrase “enhancer-promoter” means a composite unit that contains both enhancer and promoter elements. An enhancer-promoter is operatively linked to a coding sequence that encodes at least one gene product. As used herein, the phrase “operatively linked” means that an enhancer-promoter is connected to a coding sequence in such a way that the transcription of that coding sequence is controlled and regulated by that enhancer-promoter. Approaches for operatively linking an enhancer-promoter to a coding sequence are well known in the art; the precise orientation and location relative to a coding sequence of interest is dependent, inter alia, upon the specific nature of the enhancer-promoter. An enhancer-promoter used in a vector construct of the presently disclosed subject matter can be any enhancer-promoter that drives expression in a cell to be transfected. By employing an enhancer-promoter with well-known properties, the level and pattern of gene product expression can be optimized.

For introduction of, for example, a human S1R gene, a vector construct that will deliver the gene to the affected cells is desired. Viral vectors can be used. These vectors can be an adenoviral, a retroviral, a vaccinia viral vector, adeno-associated virus, or lentivirus; these vectors are preferred because they have been successfully used to deliver desired sequences to cells and tend to have a high infection efficiency. Suitable vector-S1R gene constructs are adapted for administration as pharmaceutical compositions, as described herein below. Viral promoters can also be of use in vectors of the presently disclosed subject matter, and are known in the art.

Upon a review of the instant disclosure, a therapeutically effective amount of a gene of interest is well within the reach of the skilled person. By way example with regard to dosing of adenoviral vectors, a representative dosage corresponds to at least 1×10¹² capsids/kg of body weight, at least 5×10¹² capsids/kg of body weight, or at least 1×10¹³ capsids/kg of body weight. AAV Quantification of AAV capsid particle titers is easily determined and is well known in the art (see e.g., Kohlbrenner et al., 2012; Grimm et al., 1999).

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

Representative S1R amino acid and nucleic acid sequences are disclosed in Accession No. NP_005857.1 of the GENBANK® biosequence database (223 amino acids; SEQ ID NO: 2, which is also disclosed in Accession No. Q99720 of the UniProt biosequence database) corresponding to the human S1R protein. In some embodiments, the presently disclosed subject matter provides a composition comprising a substance capable of modulating activity of S1R in a vertebrate subject. In some embodiments, the substance is selected from the group consisting of (a) a S1R polypeptide; (b) an effective amount of a siRNA that modulates expression of a S1R-encoding nucleic acid molecule, a vector encoding the siRNA, or combinations thereof; and (c) a construct comprising a nucleic acid sequence encoding a S1R polypeptide operatively linked to a promoter.

In some embodiments, the S1R polypeptide comprises a polypeptide having an amino acid sequence as set forth in SEQ ID NO: 2, which is the full length amino acid sequence of S1R. In some embodiments, fragments and homologs of SEQ ID NO: 2 are provided. In some embodiments, the nucleic acid sequence is selected from the group consisting of (a) a nucleic acid sequence encoding a polypeptide having an amino acid sequence as set forth in SEQ ID NO: 2, or a fragment or homolog thereof, (b) a nucleic acid sequence as set forth in SEQ ID NO: 1 (nucleic acid sequence encoding full length S1R) or its complementary strands; (c) a homologous nucleic acid sequence to a nucleic acid sequence as set forth in SEQ ID NO: 1, and which encodes a S1R polypeptide; and (d) a nucleic acid sequence differing from an isolated nucleic acid molecule of (a), (b), or (c) above due to degeneracy of the genetic code, and which encodes a S1R polypeptide encoded by the isolated nucleic acid molecule of (a), (b), or (c) above.

In some embodiments, the vector encoding the siRNA comprises: a promoter operatively linked to a nucleic acid molecule encoding the siRNA molecule; and a transcription termination sequence. Antisense oligonucleotide-based approaches and miRNA based approaches can also be employed.

The presently disclosed subject matter provides S1R polypeptides and biologically active fragments and homologs thereof as well as methods for preparing and testing new polypeptides for the properties disclosed herein. In some embodiments, the fragments are mammalian. In some embodiments, the fragments are human.

In some embodiments, a S1R polypeptide or biologically active fragment or homolog thereof is useful for treating inflammation, such as inflammation associated with the IRE1 pathway disclosed herein.

In some embodiments, the presently disclosed subject matter uses a biologically active S1R polypeptide or biologically active fragment or homolog thereof, or a nucleic acid sequence encoding a S1R polypeptide or a biologically active fragment or homolog thereof.

In some embodiments, the isolated polypeptide or nucleic acid sequence comprises a mammalian molecule at least about 30% homologous to a polypeptide having the amino acid sequence of at least one of the sequences disclosed herein or to a nucleic acid sequence having the amino acid sequence of at least one of the sequences disclosed herein. In some embodiments, the isolated polypeptide is at least about 35% homologous, more in some embodiments, about 40% homologous, more in some embodiments, about 45% homologous, in some embodiments, about 50% homologous, more in some embodiments, about 55% homologous, in some embodiments, about 60% homologous, more in some embodiments, about 65% homologous, in some embodiments, more in some embodiments, about 70% homologous, more in some embodiments, about 75% homologous, in some embodiments, about 80% homologous, more in some embodiments, about 85% homologous, more in some embodiments, about 90% homologous, in some embodiments, about 95% homologous, more in some embodiments, about 96% homologous, more in some embodiments, about 97% homologous, more in some embodiments, about 98% homologous, and most in some embodiments, about 99% homologous to at least one of the peptide sequences disclosed herein or to one of the nucleic acid sequences disclosed herein.

The presently disclosed subject matter further encompasses modification of the S1R and fragments thereof disclosed herein, including amino acid deletions, additions, and substitutions, particularly conservative substitutions. The presently disclosed subject matter also encompasses modifications to increase in vivo half-life and decrease degradation in vivo. Substitutions, additions, and deletions can include, for example, 1, 2, 3, 4, 5, 6, 7, 8, 9, 10, 11, 12, 13, 14, 15, 16, 17, 18, 19, 20, 21, 22, 23, 24, and 25 changes as long as the activity disclosed herein remains substantially the same.

The presently disclosed subject matter includes an isolated nucleic acid comprising a nucleic acid sequence encoding a S1R polypeptide of the presently disclosed subject matter, or a fragment or homolog thereof. In some embodiments, the nucleic acid sequence encodes a peptide comprising a S1R polypeptide sequence of the presently disclosed subject matter, or a biologically active fragment of homolog thereof.

In some embodiments, a homolog of a polypeptide (full length or fragment) of the presently disclosed subject matter is one with one or more amino acid substitutions, deletions, or additions, and with the sequence identities described herein. In some embodiments, the substitution, deletion, or addition is conservative.

In some embodiments, the subject is a mammal. In some embodiments, the mammal is a human.

The presently disclosed subject matter encompasses the use of purified isolated, recombinant, and synthetic polypeptides. The presently disclosed subject matter also provides in some embodiments recombinant nucleic acids and substantially homologous nucleic acid sequences thereto. In some embodiments, the peptide or nucleic acid is present in the pharmacologically acceptable carrier. In some embodiments, the presently disclosed polypeptides, fragments, and homologs thereof can comprise a tag sequence, linker sequence, spacer sequence and/or other additional sequence that can be used in to facilitate expression, stability, purification, isolation, or other desired feature or aspect. Multiple copies of such sequences can be employed. Such sequences can be added to the N-terminus, the C-terminus, or both of a polypeptide, fragment, or homolog thereof of the presently disclosed subject matter.

One of ordinary skill in the art will appreciate that based on the sequences of the components of the S1R polypeptides disclosed herein they can be modified independently of one another with conservative amino acid changes, including, insertions, deletions, and substitutions.

The presently disclosed subject matter further provides compositions and methods for regulating inflammation by inhibiting IRE1 activity or levels. In one aspect, IRE1 is inhibited directly. In one aspect, the inhibitor blocks an upstream stimulator of IRE1. In one aspect, the inhibitor of IRE1 activity or levels stimulates and upstream inhibitor of IRE1 activity or levels. In one aspect, an inhibitor of IRE1 is a composition comprising a substance capable of inhibiting activity of IRE1 in a vertebrate subject. In some embodiments, the substance is an oligonucleotide that modulates expression of an IRE1-encoding nucleic acid molecule, a vector encoding the oligonucleotide, or combinations thereof. In some embodiments, the IRE1-encoding nucleic acid molecule encodes a IRE1 polypeptide that comprises a polypeptide having an amino acid sequence as set forth in amino acids 19-977 of SEQ ID NO: 4, which is the amino acid sequence of the mature form of the human IRE1 polypeptide. In some embodiments, fragments and homologs of amino acids 19-977 of SEQ ID NO: 4 are provided. In some embodiments, the nucleic acid sequence is selected from the group consisting of (a) a nucleic acid sequence encoding a polypeptide having an amino acid sequence as set forth in amino acids 19-977 of SEQ ID NO: 4, or a fragment or homolog thereof, (b) a nucleic acid sequence as set forth in SEQ ID NO: 3 (nucleic acid sequence encoding full length IRE1 precursor, of which nucleotides 170-3046 encode amino acids 19-977 of SEQ ID NO: 4) or its complementary strands; (c) a homologous nucleic acid sequence to a nucleic acid sequence as set forth in SEQ ID NO: 3 (or optionally nucleotides 170-3046 thereof), and which encodes a IRE1 polypeptide; and (d) a nucleic acid sequence differing from an isolated nucleic acid molecule of (a), (b), or (c) above due to degeneracy of the genetic code, and which encodes a IRE1-encoding nucleic acid molecule polypeptide encoded by the isolated nucleic acid molecule of (a), (b), or (c) above.

In some embodiments, the vector encoding the oligonucleotide is a vector encoding a siRNA. In some embodiments the vector encoding the siRNA comprises: a promoter operatively linked to a nucleic acid molecule encoding the siRNA molecule; and a transcription termination sequence. Antisense oligonucleotide-based approaches and miRNA based approaches can also be employed.

The presently disclosed subject matter provides IRE1 polypeptides and biologically active fragments and homologs thereof as well as methods for preparing and testing new polypeptides for the properties disclosed herein. In some embodiments, the fragments are mammalian. In some embodiments, the fragments are human.

In some embodiments, the presently disclosed subject matter targets a nucleic acid sequence encoding a biologically active IRE1 polypeptide or biologically active fragment or homolog thereof. In some embodiments, the isolated polypeptide or nucleic acid sequence comprises a mammalian molecule at least about 30% homologous to a polypeptide having the amino acid sequence of at least one of the sequences disclosed herein or to a nucleic acid sequence having the amino acid sequence of at least one of the sequences disclosed herein. In some embodiments, the isolated polypeptide is at least about 35% homologous, more in some embodiments, about 40% homologous, more in some embodiments, about 45% homologous, in some embodiments, about 50% homologous, more in some embodiments, about 55% homologous, in some embodiments, about 60% homologous, more in some embodiments, about 65% homologous, in some embodiments, more in some embodiments, about 70% homologous, more in some embodiments, about 75% homologous, in some embodiments, about 80% homologous, more in some embodiments, about 85% homologous, more in some embodiments, about 90% homologous, in some embodiments, about 95% homologous, more in some embodiments, about 96% homologous, more in some embodiments, about 97% homologous, more in some embodiments, about 98% homologous, and most in some embodiments, about 99% homologous to at least one of the peptide sequences disclosed herein or to one of the nucleic acid sequences disclosed herein.

The presently disclosed subject matter further encompasses modification of the IRE1 and fragments thereof disclosed herein, including amino acid deletions, additions, and substitutions, particularly conservative substitutions.

The presently disclosed subject matter includes an isolated nucleic acid comprising a nucleic acid sequence encoding an IRE1 polypeptide of the presently disclosed subject matter, or a fragment or homolog thereof. In some embodiments, the nucleic acid sequence encodes a peptide comprising an IRE1 polypeptide sequence of the presently disclosed subject matter, or a biologically active fragment of homolog thereof.

In some embodiments, a homolog of a polypeptide (full length or fragment) of the presently disclosed subject matter is one with one or more amino acid substitutions, deletions, or additions, and with the sequence identities described herein. In some embodiments, the substitution, deletion, or addition is conservative.

In some embodiments, the subject is a mammal. In some embodiments, the mammal is a human.

The presently disclosed subject matter encompasses the use of purified isolated, recombinant, and synthetic polypeptides. The presently disclosed subject matter also provides in some embodiments recombinant nucleic acids and substantially homologous nucleic acid sequences thereto. In some embodiments, the peptide or nucleic acid is present in the pharmacologically acceptable carrier. In some embodiments, the presently disclosed polypeptides, fragments, and homologs thereof can comprise a tag sequence, linker sequence, spacer sequence and/or other additional sequence that can be used in to facilitate expression, stability, purification, isolation, or other desired feature or aspect. Multiple copies of such sequences can be employed. Such sequences can be added to the N-terminus, the C-terminus, or both of a polypeptide, fragment, or homolog thereof of the presently disclosed subject matter. The presently disclosed subject matter further encompasses modification of the IRE1 nucleic acids and fragments thereof disclosed herein, modifications to increase in vivo half-life and decrease degradation in vivo.

One of ordinary skill in the art will appreciate that based on the sequences of the components of the IRE1 polypeptides and nucleic acid sequences disclosed herein they can be modified independently of one another with conservative amino acid changes, including, insertions, deletions, and substitutions.

Peptide Modification and Preparation

Peptide preparation is described in the Examples. It will be appreciated, of course, that the proteins or peptides of the presently disclosed subject matter may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C1-C5 branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH₂), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Acid addition salts of the presently disclosed subject matter are also contemplated as functional equivalents. Thus, a peptide in accordance with the presently disclosed subject matter treated with an inorganic acid such as hydrochloric, hydrobromic, sulfuric, nitric, phosphoric, and the like, or an organic acid such as an acetic, propionic, glycolic, pyruvic, oxalic, malic, malonic, succinic, maleic, fumaric, tataric, citric, benzoic, cinnamie, mandelic, methanesulfonic, ethanesulfonic, p-toluenesulfonic, salicyclic and the like, to provide a water soluble salt of the peptide is suitable for use in the presently disclosed subject matter.

The presently disclosed subject matter also provides for analogs of proteins. Analogs can differ from naturally occurring proteins or peptides by conservative amino acid sequence differences or by modifications which do not affect sequence, or by both. For example, conservative amino acid changes may be made, which although they alter the primary sequence of the protein or peptide, do not normally alter its function. To that end, 10 or more conservative amino acid changes typically have no effect on peptide function.

Modifications (which do not normally alter primary sequence) include in vivo, or in vitro chemical derivatization of polypeptides, e.g., acetylation, or carboxylation. Also included are modifications of glycosylation, e.g., those made by modifying the glycosylation patterns of a polypeptide during its synthesis and processing or in further processing steps; e.g., by exposing the polypeptide to enzymes which affect glycosylation, e.g., mammalian glycosylating or deglycosylating enzymes. Also embraced are sequences which have phosphorylated amino acid residues, e.g., phosphotyrosine, phosphoserine, or phosphothreonine.

Also included are polypeptides which have been modified using ordinary molecular biological techniques so as to improve their resistance to proteolytic degradation or to optimize solubility properties or to render them more suitable as a therapeutic agent.

Analogs of such polypeptides include those containing residues other than naturally occurring L-amino acids, e.g., D-amino acids or non-naturally occurring or non-standard synthetic amino acids. The peptides of the presently disclosed subject matter are not limited to products of any of the specific exemplary processes listed herein.

The presently disclosed subject matter includes the use of beta-alanine (also referred to as β-alanine, β-Ala, bA, and βA, having the structure:

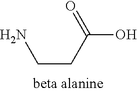

It will be appreciated, of course, that the polypeptides, derivatives, or fragments thereof may incorporate amino acid residues which are modified without affecting activity. For example, the termini may be derivatized to include blocking groups, i.e. chemical substituents suitable to protect and/or stabilize the N- and C-termini from “undesirable degradation”, a term meant to encompass any type of enzymatic, chemical or biochemical breakdown of the compound at its termini which is likely to affect the function of the compound, i.e. sequential degradation of the compound at a terminal end thereof.

Blocking groups include protecting groups conventionally used in the art of peptide chemistry which will not adversely affect the in vivo activities of the peptide. For example, suitable N-terminal blocking groups can be introduced by alkylation or acylation of the N-terminus. Examples of suitable N-terminal blocking groups include C1-C5 branched or unbranched alkyl groups, acyl groups such as formyl and acetyl groups, as well as substituted forms thereof, such as the acetamidomethyl (Acm) group. Desamino analogs of amino acids are also useful N-terminal blocking groups, and can either be coupled to the N-terminus of the peptide or used in place of the N-terminal reside. Suitable C-terminal blocking groups, in which the carboxyl group of the C-terminus is either incorporated or not, include esters, ketones or amides. Ester or ketone-forming alkyl groups, particularly lower alkyl groups such as methyl, ethyl and propyl, and amide-forming amino groups such as primary amines (—NH2), and mono- and di-alkylamino groups such as methylamino, ethylamino, dimethylamino, diethylamino, methylethylamino and the like are examples of C-terminal blocking groups. Descarboxylated amino acid analogues such as agmatine are also useful C-terminal blocking groups and can be either coupled to the peptide's C-terminal residue or used in place of it. Further, it will be appreciated that the free amino and carboxyl groups at the termini can be removed altogether from the peptide to yield desamino and descarboxylated forms thereof without affect on peptide activity.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

Substantially pure protein obtained as described herein may be purified by following known procedures for protein purification, wherein an immunological, enzymatic or other assay is used to monitor purification at each stage in the procedure. Protein purification methods are well known in the art, and are described, for example in Deutscher et al., 1990.

As discussed, modifications or optimizations of peptide ligands of the presently disclosed subject matter are within the scope of the application. Modified or optimized peptides are included within the definition of peptide binding ligand. Specifically, a peptide sequence identified can be modified to optimize its potency, pharmacokinetic behavior, stability and/or other biological, physical and chemical properties.

### III.C. Amino Acid Substitutions

In certain embodiments, the disclosed methods and compositions may involve preparing polypeptides with one or more substituted amino acid residues.

In various embodiments, the structural, physical and/or therapeutic characteristics of peptide sequences may be optimized by replacing one or more amino acid residues.

Other modifications can also be incorporated without adversely affecting the activity and these include, but are not limited to, substitution of one or more of the amino acids in the natural L-isomeric form with amino acids in the D-isomeric form. Thus, the peptide may include one or more D-amino acid resides, or may comprise amino acids which are all in the D-form. Retro-inverso forms of peptides in accordance with the presently disclosed subject matter are also contemplated, for example, inverted peptides in which all amino acids are substituted with D-amino acid forms.

The skilled artisan will be aware that, in general, amino acid substitutions in a peptide typically involve the replacement of an amino acid with another amino acid of relatively similar properties (i.e., conservative amino acid substitutions). The properties of the various amino acids and effect of amino acid substitution on protein structure and function have been the subject of extensive study and knowledge in the art.

For example, one can make the following isosteric and/or conservative amino acid changes in the parent polypeptide sequence with the expectation that the resulting polypeptides would have a similar or improved profile of the properties described above:

Substitution of alkyl-substituted hydrophobic amino acids: including alanine, leucine, isoleucine, valine, norleucine, S-2-aminobutyric acid, S-cyclohexylalanine or other simple alpha-amino acids substituted by an aliphatic side chain from C1-10 carbons including branched, cyclic and straight chain alkyl, alkenyl or alkynyl substitutions.

Substitution of aromatic-substituted hydrophobic amino acids: including phenylalanine, tryptophan, tyrosine, biphenylalanine, 1-naphthylalanine, 2-naphthylalanine, 2-benzothienylalanine, 3-benzothienylalanine, histidine, amino, alkylamino, dialkylamino, aza, halogenated (fluoro, chloro, bromo, or iodo) or alkoxy-substituted forms of the previous listed aromatic amino acids, illustrative examples of which are: 2-,3- or 4-aminophenylalanine, 2-,3- or 4-chlorophenylalanine, 2-,3- or 4-methylphenylalanine, 2-,3- or 4-methoxyphenylalanine, 5-amino-, 5-chloro-, 5-methyl- or 5-methoxytryptophan, 2′-, 3′-, or 4′-amino-, 2′-, 3′-, or 4′-chloro-, 2,3, or 4-biphenylalanine, 2′,-3′,- or 4′-methyl-2, 3 or 4-biphenylalanine, and 2- or 3-pyridylalanine.

Substitution of amino acids containing basic functions: including arginine, lysine, histidine, ornithine, 2,3-diaminopropionic acid, homoarginine, alkyl, alkenyl, or aryl-substituted (from C1-C10 branched, linear, or cyclic) derivatives of the previous amino acids, whether the substituent is on the heteroatoms (such as the alpha nitrogen, or the distal nitrogen or nitrogens, or on the alpha carbon, in the pro-R position for example. Compounds that serve as illustrative examples include: N-epsilon-isopropyl-lysine, 3-(4-tetrahydropyridyl)-glycine, 3-(4-tetrahydropyridyl)-alanine, N,N-gamma, gamma′-diethyl-homoarginine. Included also are compounds such as alpha methyl arginine, alpha methyl 2,3-diaminopropionic acid, alpha methyl histidine, alpha methyl ornithine where alkyl group occupies the pro-R position of the alpha carbon. Also included are the amides formed from alkyl, aromatic, heteroaromatic (where the heteroaromatic group has one or more nitrogens, oxygens, or sulfur atoms singly or in combination) carboxylic acids or any of the many well-known activated derivatives such as acid chlorides, active esters, active azolides and related derivatives) and lysine, ornithine, or 2,3-diaminopropionic acid.

Substitution of acidic amino acids: including aspartic acid, glutamic acid, homoglutamic acid, tyrosine, alkyl, aryl, arylalkyl, and heteroaryl sulfonamides of 2,4-diaminopriopionic acid, ornithine or lysine and tetrazole-substituted alkyl amino acids.

Substitution of side chain amide residues: including asparagine, glutamine, and alkyl or aromatic substituted derivatives of asparagine or glutamine.

Substitution of hydroxyl containing amino acids: including serine, threonine, homoserine, 2,3-diaminopropionic acid, and alkyl or aromatic substituted derivatives of serine or threonine. It is also understood that the amino acids within each of the categories listed above can be substituted for another of the same group.

For example, the hydropathic index of amino acids may be considered (Kyte & Doolittle, 1982, J. Mol. Biol., 157:105-132). The relative hydropathic character of the amino acid contributes to the secondary structure of the resultant protein, which in turn defines the interaction of the protein with other molecules. Each amino acid has been assigned a hydropathic index on the basis of its hydrophobicity and charge characteristics (Kyte & Doolittle, 1982), these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). In making conservative substitutions, the use of amino acids whose hydropathic indices are within +/−2 is preferred, within +/−1 are more preferred, and within +1-0.5 are even more preferred.

Amino acid substitution may also take into account the hydrophilicity of the amino acid residue (e.g., U.S. Pat. No. 4,554,101). Hydrophilicity values have been assigned to amino acid residues: arginine (+3.0); lysine (+3.0); aspartate (+3.0); glutamate (+3.0); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). Replacement of amino acids with others of similar hydrophilicity is preferred.

Other considerations include the size of the amino acid side chain. For example, it would generally not be preferred to replace an amino acid with a compact side chain, such as glycine or serine, with an amino acid with a bulky side chain, e.g., tryptophan or tyrosine. The effect of various amino acid residues on protein secondary structure is also a consideration. Through empirical study, the effect of different amino acid residues on the tendency of protein domains to adopt an alpha-helical, beta-sheet or reverse turn secondary structure has been determined and is known in the art (see e.g., Chou & Fasman, 1974; Chou & Fasman, 1978; Chou & Fasman, 1979).

Based on such considerations and extensive empirical study, tables of conservative amino acid substitutions have been constructed and are known in the art. For example: arginine and lysine; glutamate and aspartate; serine and threonine; glutamine and asparagine; and valine, leucine and isoleucine. Alternatively: Ala (A) Leu, Ile, Val; Arg (R) Gln, Asn, Lys; Asn (N) His, Asp, Lys, Arg, Gln; Asp (D) Asn, Glu; Cys (C) Ala, Ser; Gln (Q) Glu, Asn; Glu (E) Gln, Asp; Gly (G) Ala; His (H) Asn, Gln, Lys, Arg; Ile (I) Val, Met, Ala, Phe, Leu; Leu (L) Val, Met, Ala, Phe, Ile; Lys (K) Gln, Asn, Arg; Met (M) Phe, Ile, Leu; Phe (F) Leu, Val, Ile, Ala, Tyr; Pro (P) Ala; Ser (S), Thr; Thr (T) Ser; Trp (W) Phe, Tyr; Tyr (Y) Trp, Phe, Thr, Ser; Val (V) Ile, Leu, Met, Phe, Ala.

Other considerations for amino acid substitutions include whether or not the residue is located in the interior of a protein or is solvent exposed. For interior residues, conservative substitutions would include: Asp and Asn; Ser and Thr; Ser and Ala; Thr and Ala; Ala and Gly; Ile and Val; Val and Leu; Leu and Ile; Leu and Met; Phe and Tyr; Tyr and Trp. (See e.g., PROWL Rockefeller University website). For solvent exposed residues, conservative substitutions would include: Asp and Asn; Asp and Glu; Glu and Gln; Glu and Ala; Gly and Asn; Ala and Pro; Ala and Gly; Ala and Ser; Ala and Lys; Ser and Thr; Lys and Arg; Val and Leu; Leu and Ile; Ile and Val; Phe and Tyr. Various matrices have been constructed to assist in selection of amino acid substitutions, such as the PAM250 scoring matrix, Dayhoff matrix, Grantham matrix, McLachlan matrix, Doolittle matrix, Henikoff matrix, Miyata matrix, Fitch matrix, Jones matrix, Rao matrix, Levin matrix and Risler matrix (Idem.)

In determining amino acid substitutions, one may also consider the existence of intermolecular or intramolecular bonds, such as formation of ionic bonds (salt bridges) between positively charged residues (e.g., His, Arg, Lys) and negatively charged residues (e.g., Asp, Glu) or disulfide bonds between nearby cysteine residues.

Methods of substituting any amino acid for any other amino acid in an encoded peptide sequence are well known and a matter of routine experimentation for the skilled artisan, for example by the technique of site-directed mutagenesis or by synthesis and assembly of oligonucleotides encoding an amino acid substitution and splicing into an expression vector construct.

### III.D. Pharmaceutical Compositions and Administration

The presently disclosed subject matter is also directed to methods of administering the compounds of the presently disclosed subject matter to a subject.

Pharmaceutical compositions comprising the present compounds are administered to a subject in need thereof by any number of routes including, but not limited to, topical, oral, intravenous, intramuscular, intra-arterial, intramedullary, intrathecal, intraventricular, transdermal, subcutaneous, intraperitoneal, intranasal, enteral, topical, sublingual, or rectal approaches.

In accordance with one embodiment, a method for treating a subject in need of such treatment is provided. The method comprises administering a pharmaceutical composition comprising at least one composition of the presently disclosed subject matter to a subject in need thereof. Compositions provided by the methods of the presently disclosed subject matter can be administered with known compounds or other medications as well.

The pharmaceutical compositions useful for practicing the presently disclosed subject matter may be administered to deliver a dose of between 1 ng/kg/day and 100 mg/kg/day.

The presently disclosed subject matter encompasses the preparation and use of pharmaceutical compositions comprising a compound useful for treatment of the diseases and disorders disclosed herein as an active ingredient. Such a pharmaceutical composition may consist of the active ingredient alone, in a form suitable for administration to a subject, or the pharmaceutical composition may comprise the active ingredient and one or more pharmaceutically acceptable carriers, one or more additional ingredients, or some combination of these. The active ingredient may be present in the pharmaceutical composition in the form of a physiologically acceptable ester or salt, such as in combination with a physiologically acceptable cation or anion, as is well known in the art.

As used herein, the term “physiologically acceptable” ester or salt means an ester or salt form of the active ingredient which is compatible with any other ingredients of the pharmaceutical composition, which is not deleterious to the subject to which the composition is to be administered.

The compositions of the presently disclosed subject matter may comprise at least one active polypeptide, one or more acceptable carriers, and optionally other polypeptides or therapeutic agents.

For in vivo applications, the compositions of the presently disclosed subject matter may comprise a pharmaceutically acceptable salt. Suitable acids which are capable of forming such salts with the compounds of the presently disclosed subject matter include inorganic acids such as hydrochloric acid, hydrobromic acid, perchloric acid, nitric acid, thiocyanic acid, sulfuric acid, phosphoric acid and the like; and organic acids such as formic acid, acetic acid, propionic acid, glycolic acid, lactic acid, anthranilic acid, cinnamic acid, naphthalene sulfonic acid, sulfanilic acid and the like.

Pharmaceutically acceptable carriers include physiologically tolerable or acceptable diluents, excipients, solvents, or adjuvants. The compositions are in some embodiments sterile and nonpyrogenic. Examples of suitable carriers include, but are not limited to, water, normal saline, dextrose, mannitol, lactose or other sugars, lecithin, albumin, sodium glutamate, cysteine hydrochloride, ethanol, polyols (propylene glycol, polyethylene glycol, glycerol, and the like), vegetable oils (such as olive oil), injectable organic esters such as ethyl oleate, ethoxylated isosteraryl alcohols, polyoxyethylene sorbitol and sorbitan esters, microcrystalline cellulose, aluminum methahydroxide, bentonite, kaolin, agar-agar and tragacanth, or mixtures of these substances, and the like.

The pharmaceutical compositions may also contain minor amounts of nontoxic auxiliary pharmaceutical substances or excipients and/or additives, such as wetting agents, emulsifying agents, pH buffering agents, antibacterial and antifungal agents (such as parabens, chlorobutanol, phenol, sorbic acid, and the like). Suitable additives include, but are not limited to, physiologically biocompatible buffers (e.g., tromethamine hydrochloride), additions (e.g., 0.01 to 10 mole percent) of chelants (such as, for example, DTPA or DTPA-bisamide) or calcium chelate complexes (as for example calcium DTPA or CaNaDTPA-bisamide), or, optionally, additions (e.g., 1 to 50 mole percent) of calcium or sodium salts (for example, calcium chloride, calcium ascorbate, calcium gluconate or calcium lactate). If desired, absorption enhancing or delaying agents (such as liposomes, aluminum monostearate, or gelatin) may be used. The compositions can be prepared in conventional forms, either as liquid solutions or suspensions, solid forms suitable for solution or suspension in liquid prior to injection, or as emulsions. Pharmaceutical compositions according to the presently disclosed subject matter can be prepared in a manner fully within the skill of the art.

The compositions of the presently disclosed subject matter or pharmaceutical compositions comprising these compositions may be administered so that the compositions may have a physiological effect. Administration may occur enterally or parenterally; for example, orally, rectally, intracisternally, intravaginally, intraperitoneally, locally (e.g., with powders, ointments or drops), or as a buccal or nasal spray or aerosol. Parenteral administration is an approach. Particular parenteral administration methods include intravascular administration (e.g., intravenous bolus injection, intravenous infusion, intra-arterial bolus injection, intra-arterial infusion and catheter instillation into the vasculature), peri- and intra-target tissue injection, subcutaneous injection or deposition including subcutaneous infusion (such as by osmotic pumps), intramuscular injection, and direct application to the target area, for example by a catheter or other placement device.

Where the administration of the composition is by injection or direct application, the injection or direct application may be in a single dose or in multiple doses. Where the administration of the compound is by infusion, the infusion may be a single sustained dose over a prolonged period of time or multiple infusions.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

It will be understood by the skilled artisan that such pharmaceutical compositions are generally suitable for administration to animals of all sorts. Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, birds including commercially relevant birds such as chickens, ducks, geese, and turkeys.

A pharmaceutical composition of the presently disclosed subject matter may be prepared, packaged, or sold in bulk, as a single unit dose, or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject or a convenient fraction of such a dosage such as, for example, one-half or one-third of such a dosage.

The relative amounts of the active ingredient, the pharmaceutically acceptable carrier, and any additional ingredients in a pharmaceutical composition of the presently disclosed subject matter will vary, depending upon the identity, size, and condition of the subject treated and further depending upon the route by which the composition is to be administered. By way of example, the composition may comprise between 0.1% and 100% (w/w) active ingredient.

In addition to the active ingredient, a pharmaceutical composition of the presently disclosed subject matter may further comprise one or more additional pharmaceutically active agents. Particularly contemplated additional agents include anti-emetics and scavengers such as cyanide and cyanate scavengers.

Controlled- or sustained-release formulations of a pharmaceutical composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Gennaro, 1990 and/or Gennaro, 2003, each of which is incorporated herein by reference.

Typically, dosages of the compound of the presently disclosed subject matter which may be administered to an animal, in some embodiments a human, range in amount from 1 μg to about 100 g per kilogram of body weight of the animal. While the precise dosage administered will vary depending upon any number of factors, including but not limited to, the type of animal and type of disease state being treated, the age of the animal and the route of administration. In some embodiments, the dosage of the compound will vary from about 1 mg to about 10 g per kilogram of body weight of the animal. In another aspect, the dosage will vary from about 10 mg to about 1 g per kilogram of body weight of the animal.

The compositions may be administered to an animal as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type of cancer being diagnosed, the type and severity of the condition or disease being treated, the type and age of the animal, etc.

Suitable preparations include injectables, either as liquid solutions or suspensions, however, solid forms suitable for solution in, suspension in, liquid prior to injection, may also be prepared. The preparation may also be emulsified, or the compositions encapsulated in liposomes. The active ingredients are often mixed with excipients which are pharmaceutically acceptable and compatible with the active ingredient. Suitable excipients are, for example, water saline, dextrose, glycerol, ethanol, or the like and combinations thereof. In addition, if desired, the preparation may also include minor amounts of auxiliary substances such as wetting or emulsifying agents, pH buffering agents, and/or adjuvants.

The presently disclosed subject matter also includes a kit comprising the composition of the presently disclosed subject matter and an instructional material which describes adventitially administering the composition to a cell or a tissue of a subject. In some embodiments, this kit comprises a (in some embodiments sterile) solvent suitable for dissolving or suspending the composition of the presently disclosed subject matter prior to administering the compound to the subject and/or a device suitable for administering the composition such as a syringe, injector, or the like or other device as would be apparent to one of ordinary skill in the art upon a review of the instant disclosure.

As used herein, an “instructional material” includes a publication, a recording, a diagram, or any other medium of expression which can be used to communicate the usefulness of the peptide of the presently disclosed subject matter in the kit for effecting alleviation of the various diseases or disorders recited herein. Optionally, or alternately, the instructional material may describe one or more methods of using the compositions for diagnostic or identification purposes or of alleviation the diseases or disorders in a cell or a tissue of a mammal. The instructional material of the kit of the presently disclosed subject matter may, for example, be affixed to a container which contains the multimeric peptide of the presently disclosed subject matter or be shipped together with a container which contains the peptide. Alternatively, the instructional material may be shipped separately from the container with the intention that the instructional material and the composition be used cooperatively by the recipient.

Various aspects and embodiments of the presently disclosed subject matter are described in further detail below.

Compounds binding to the sigma receptor as defined herein, may be antagonists, inverse agonists, agonists, partial antagonists, and/or partial agonists.

Methods and concentrations for dosing with compounds such as ANAVEX® 2-73 are provided herein or are known in the art (although for other uses), for example, see U.S. Patent Application Publication No. 2014/0296211, incorporated herein by reference in its entirety.

Examples of other potentially useful compounds, include, but are not limited to, ANAVEX-27-1041, PRS-013, SA-4503, siramesine, ANAVEX-7-1037, (+)-pentazocine, (+)-SKF 10,047, PRE084 (2-morpholin-4-yl-ethyl 1-phenylcyclohexane-1-carboxylate), SA4503 (1-[2-(3,4-dimethoxyphenyl)ethyl]-4-β-phenylpropyl)piperazine), (±)-PPCC oxalate, PRE-084 hydrochloride, SA 4503 dihydrochloride, and (+)-SK&F 10047 hydrochloride.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

The compounds of the presently disclosed subject matter may be administered to, for example, a cell, a tissue, or a subject by any of several methods described herein and by others which are known to those of skill in the art.

The relative amounts of the active ingredient, the pharmaceutically acceptable carrier, and any additional ingredients in a pharmaceutical composition of the presently disclosed subject matter will vary, depending upon the identity, sex, age, size, and condition of the subject treated and further depending upon the route by which the composition is to be administered.

In addition to the active ingredient, a composition of the presently disclosed subject matter may further comprise one or more additional pharmaceutically active or therapeutic agents. Particularly contemplated additional agents include anti-emetics and scavengers such as cyanide and cyanate scavengers.

Controlled- or sustained-release formulations of a composition of the presently disclosed subject matter may be made using conventional technology.

As used herein, “additional ingredients” include, but are not limited to, one or more of the following: excipients; surface active agents; dispersing agents; inert diluents; granulating and disintegrating agents; binding agents; lubricating agents; sweetening agents; flavoring agents; coloring agents; preservatives; physiologically degradable compositions such as gelatin; aqueous vehicles and solvents; oily vehicles and solvents; suspending agents; dispersing or wetting agents; emulsifying agents, demulcents; buffers; salts; thickening agents; fillers; emulsifying agents; antioxidants; antibiotics; antifungal agents; stabilizing agents; and pharmaceutically acceptable polymeric or hydrophobic materials. Other “additional ingredients” which may be included in the pharmaceutical compositions of the presently disclosed subject matter are known in the art and described, for example in Gennaro, 1990 and/or Gennaro, 2003, each of which is incorporated herein by reference.

Other components such as preservatives, antioxidants, surfactants, absorption enhancers, viscosity enhancers or film forming polymers, bulking agents, diluents, coloring agents, flavoring agents, pH modifiers, sweeteners or taste-masking agents may also be incorporated into the composition. Suitable coloring agents include red, black, and yellow iron oxides and FD&C dyes such as FD&C Blue No. 2, FD&C Red No. 40, and the like. Suitable flavoring agents include mint, raspberry, licorice, orange, lemon, grapefruit, caramel, vanilla, cherry grape flavors, combinations thereof, and the like. Suitable pH modifiers include citric acid, tartaric acid, phosphoric acid, hydrochloric acid, maleic acid, sodium hydroxide, and the like. Suitable sweeteners include aspartame, acesulfame K, thaumatic, and the like. Suitable taste-masking agents include sodium bicarbonate, ion-exchange resins, cyclodextrin inclusion compounds, adsorbates, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

Although the descriptions of pharmaceutical compositions provided herein are principally directed to pharmaceutical compositions which are suitable for ethical administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and perform such modification with merely ordinary, if any, experimentation. Subjects to which administration of the pharmaceutical compositions of the presently disclosed subject matter is contemplated include, but are not limited to, humans and other primates, mammals including commercially relevant mammals such as cattle, pigs, horses, sheep, cats, and dogs, and birds including commercially relevant birds such as chickens, ducks, geese, and turkeys.

The pharmaceutical compositions of the presently disclosed subject matter can be administered in any suitable formulation, by any suitable means, and by any suitable route of administration. Formulations suitable for topical administration include, but are not limited to, liquid or semi-liquid preparations such as liniments, lotions, oil in water or water in oil emulsions such as creams, ointments or pastes, and solutions or suspensions. Topically-administrable formulations may, for example, comprise from about 1% to about 10% (w/w) active ingredient, although the concentration of the active ingredient may be as high as the solubility limit of the active ingredient in the solvent. Formulations for topical administration may further comprise one or more of the additional ingredients described herein.

In accordance with the presently disclosed subject matter, as described above or as discussed in the Examples below, there can be employed conventional chemical, cellular, histochemical, biochemical, molecular biology, microbiology, recombinant DNA, and clinical techniques which are known to those of skill in the art. Such techniques are explained fully in the literature. See for example, Sambrook et al., 1989; Glover, 1985; Gait, 1984; Harlow & Lane, 1988; Roe et al., 1996; and Ausubel et al., 1995.

The presently disclosed subject matter may be embodied in other specific forms without departing from the spirit or essential attributes thereof. The presently disclosed subject matter encompasses all combinations of the different aspects of the presently disclosed subject matter noted herein. It is understood that any and all embodiments of the presently disclosed subject matter may be taken in conjunction with any other embodiment or embodiments to describe additional more preferred embodiments. It is also to be understood that each individual element of the preferred embodiments is intended to be taken individually as its own independent preferred embodiment. Furthermore, any element of an embodiment is meant to be combined with any and all other elements from any embodiment to describe an additional embodiment.

Typically, dosages of the compounds of the presently disclosed subject matter which may be administered to an animal, preferably a human, range in amount from about 1.0 μg to about 100 g per kilogram of body weight of the animal. The precise dosage administered will vary depending upon any number of factors, including but not limited to, the type of animal and type of disease state being treated, the age of the animal and the route of administration. Preferably, the dosage of the compound will vary from about 1 mg to about 10 g per kilogram of body weight of the animal. More preferably, the dosage will vary from about 10 mg to about 1 g per kilogram of body weight of the animal.

The compounds may be administered to a subject as frequently as several times daily, or it may be administered less frequently, such as once a day, once a week, once every two weeks, once a month, or even less frequently, such as once every several months or even once a year or less. The frequency of the dose will be readily apparent to the skilled artisan and will depend upon any number of factors, such as, but not limited to, the type and severity of the disease being treated, the type and age of the animal, etc.

### EXAMPLES

The presently disclosed subject matter will be now be described more fully hereinafter with reference to the accompanying EXAMPLES, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

### Materials and Methods for Examples 1-7

Mice. The S1R knockout mouse strain B6.129S5-Sigmar1^{Gt(OST422756)Lex}/Mmucd was acquired from MMRRC (Sabino et al., 2009; Ha et al., 2011). All animal experiments were approved and complied with regulations of the Institutional Animal Care and Use Committee at University of Virginia.

Endotoxin challenge. In vivo endotoxin challenge was performed on mice at 8-12 weeks of age. LPS from E. coli 0111:B4 (Sigma-Aldrich, L2630) was injected intraperitoneally as described in the manuscript. STF 083010 (Medchem Express, HY-15845) was resuspended in 33% Kolliphor-EL (Sigma, C5135) and administered intraperitoneally at 30 mg/kg immediately after and again 24 hours after LPS injection.

Tissue culture conditions and Reagents. HEK293 cells, primary lung fibroblasts and BMDM were isolated and maintained as described (41-43). Cells were treated with LPS (Sigma, L4391), 4μ8C (Tocris, 4479), tunicamycin (Tocris, 3516), PD98059 (Medchem Express HY-12028), JSH-23 (Medchem Express, Hy-13982), SP600125 (Medchem Express, HY-12041), cycloheximide (Tocris, 0970), lactacystin (Cayman Chemical, 70980), chloroquine (Fisher, ICN19391925), and STF 083010 (Medchem Express, HY-15845) as described in the text.

ELISA. ELISA for IL-6 and TNFα were performed as previously described (Remick et al., 2002). Antibodies used were: anti-mouse IL-6 MP5-20F3 (Biolegend, 504501) 0.5 μg/mL; biotin anti-mouse IL-6 MP5-32C₁₁ (Biolegend, 504601) 1 μg/mL; anti-mouse TNFα (R&D systems, AF-410-NA) 0.5 μg/mL; biotin anti-mouse TNFα (R&D systems, BAF410) 0.25 μg/mL.

Peritoneal contents collection. Three hours after LPS injection, peritoneal cavities were washed with 2.5 mL of ice-cold PBS+5 mM EDTA, and then centrifuged to pellet cells. Supernatants were collected for ELISA, and the remaining cells were washed and stored as previously described (Gaultier et al., 2008).

Western blot. Protein extraction and western blot were performed as previously described (Gaultier et al., 2010). Antibodies were used according to manufacturer's instruction: Bax (CST, 14796), Bcl-2 (CST, 2870), FLAG (Sigma, F7425) 1:1000; total IkBα (CST, 4814) 1:1000; total ERK1/2 (CST, 9102) 1:1000; phospho-ERK1/2 (CST, 4370) 1:1000; total IRE1α (CST, 3294) 1:1000; phospho-IRE1α (Abcam, 48187) 1:1000; total JNK (CST, 9252) 1:1000; phospho-JNK (CST, 9251) 1:1000; total p65 NF-κB (CST, 8242) 1:1000; phospho p65 NF-κB (CST, 3033) 1:1000; sigma-1 receptor (Santa Cruz, 137075) 1:500. Linear level adjustments were applied to entire images to enhance visualization.

Immunofluorescence. Cells were fixed in 4% PFA and stained with anti-p65 NF-κB at 1:250, then goat anti rabbit ALEXAFLUOR® 546 (Thermo, A-11035) at 1:1000. Slides were mounted in ProLong Gold (Thermo, P36931) and imaged with an EVOS FL Auto Imaging System.

Transfection of proteins for overexpression. HEK293 cells were transfected using XTremegene HP transfection reagent (Roche, 06366244001). Overexpression plasmids used were the following: IRE1 alpha-pcDNA3.EGFP (Addgene #13009; Lipson et al., 2006), MESD-FLAG (Hsieh et al., 2003), MGC Mouse Sigmar1 cDNA (GE Life Sciences, MMM1013-202768624).

cDNA synthesis and quantitative RT-PCR. Total RNA was extracted using an ISOLATE II RNA (Bioline, 52073) and cDNA synthesis was performed with the ISCRIPT™ kit (Bio-Rad, 170-8891). TaqMan Probes were obtained from Life Technologies (GAPDH: Mm99999915_g1; IL-6: Mm00446190_m1; pro-IL-1β: Mm00434228_m1; TNFα: Mm00443258_m1). Primers for IRE1 detection were GCACGUGAAUUGAUAGAGA (SEQ ID NO: 10) and UCUCUAUCAAUUCACGUGC (SEQ ID NO: 11). Primers for the detection of XBP1 were previously published (Lipson et al., 2008). RT-PCR was performed as described previously (Gaultier et al., 2009).

Data analysis and statistics. Densitometry was performed using ImageJ software. Statistical analyses, as indicated in each figure legend, were performed using GraphPad Prism.

### Example 1

### Lack of S1R Increases Susceptibility to Septic Shock

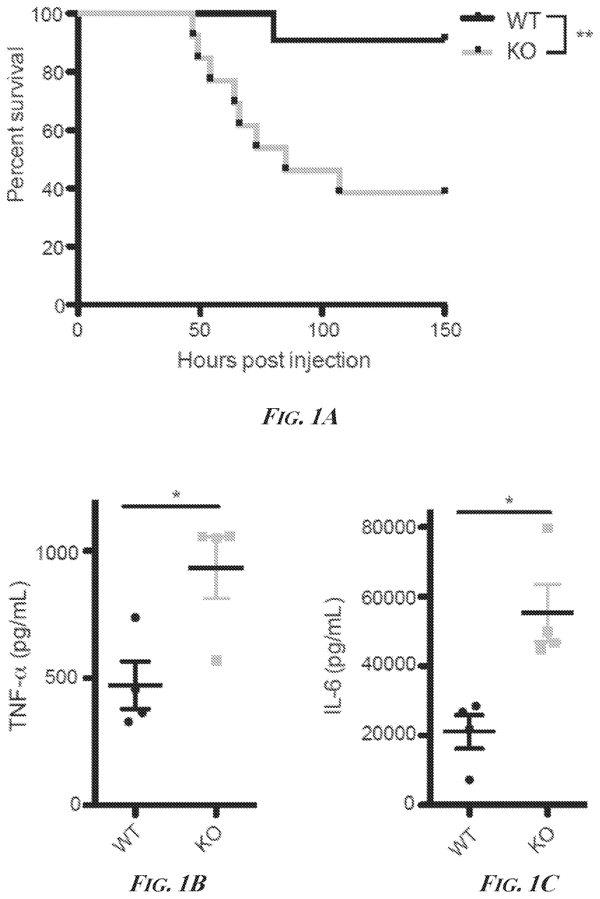

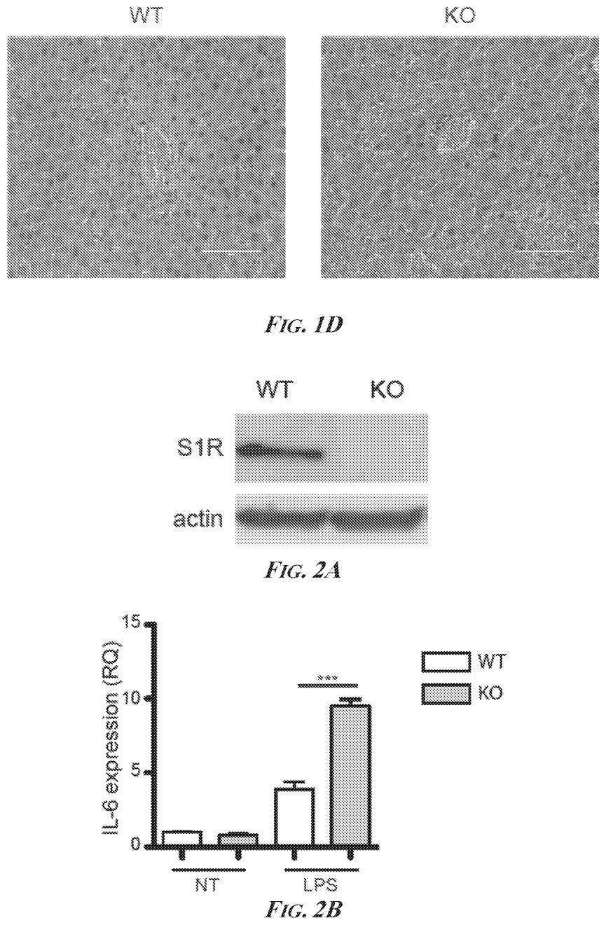

To begin to understand the function of S1R during the inflammatory response, we used LPS challenge, a well-accepted model of septic shock. Control (WT) and S1R knockout (KO) mice were injected with a sub-lethal dose of LPS (5 mg/kg) and survival was monitored for 6 days. While WT animals, as expected, experienced very low mortality (9%), 62% of S1R KO mice succumbed to LPS induced death (FIG. 1A). To determine the potential cause of mortality in S1R KO, we analyzed the circulating levels of pro-inflammatory cytokines TNF-α and IL-6 at the early time points of the inflammatory response. For both cytokines, degree of induction at early time points has been shown to correlate with mortality during sepsis (Remick et al., 2002; Copeland et al., 2005). We found that the serum levels of TNF-α and IL-6 at 90 and 180 min, respectively, were significantly increased in the S1R KO mice, when compared to controls (FIGS. 1B and 1C). Importantly, TNF-α and IL-6 were not detectable in the serum of unchallenged S1R KO mice, suggesting that S1R specifically regulates LPS induced secretion of these cytokines. In support of the hypothesis that LPS challenged S1R KO mice likely perish due to the development of a cytokine storm and septic shock, we found no evidence of increased liver necrosis in S1R KO mice, when compared to controls (FIG. 1D). These data collectively show that the lack of S1R results in increased pro-inflammatory cytokine response and LPS-induced mortality.

### Example 2

### S1R Deficient Macrophages have Increased Production of Inflammatory Cytokines

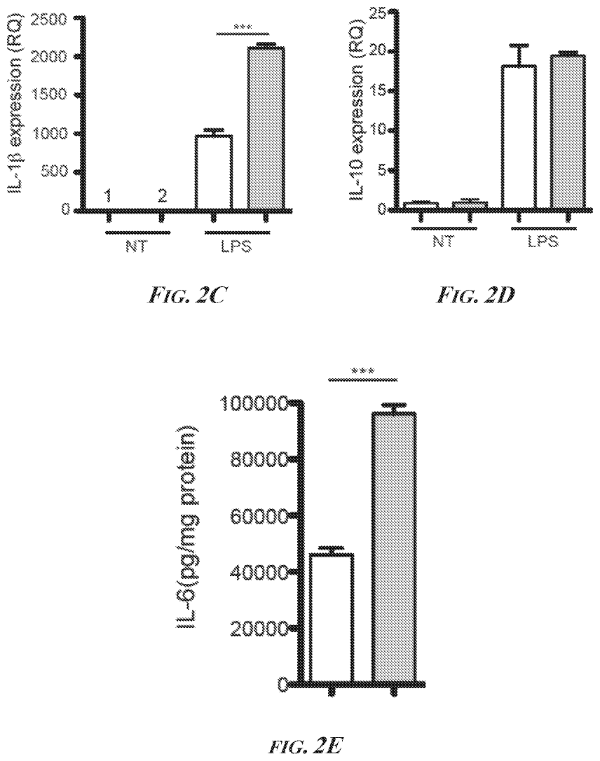

During sepsis and LPS challenge, macrophages are the major producers of inflammatory cytokines, and thus they are well suited for the mechanistic dissection of the inflammatory response to LPS (Glauser et al., 1991). We prepared bone marrow derived macrophages (BMDM) from WT and S1R KO mice and examined expression of S1R at the protein level by immunoblot analysis of the cellular lysates. As expected, S1R expression is observed in WT, but not in S1R KO BMDM (FIG. 2A). Next, using qPCR, we examined expression of pro-inflammatory cytokines in BMDM exposed to LPS. Analysis of IL-6 and IL-1β transcripts revealed that S1R deficient cells had significantly increased expression of these inflammatory cytokines, when compared to control BMDM (FIGS. 2B and 2C). Importantly, S1R deficient cells do not exhibit an indiscriminate cytokine expression increase, as we did not observe elevated levels of the transcript encoding IL-10 upon LPS stimulation of WT and S1R KO BMDM (FIG. 2D). We also monitored IL-6 protein levels in the supernatants of LPS treated BMDM by ELISA. In agreement with our qPCR results, S1R deficient BMDM secreted significantly more IL-6 than WT BMDM (FIG. 2E). This effect was not limited to IL-6, as revealed by multiplex analysis of cytokine expression; IL-1α, IL-1β, IL-12 and TNF-α were also elevated in S1R KO BMDM after LPS treatment (FIG. 2F). Therefore, S1R deficiency in macrophages is associated with increased production of pro-inflammatory cytokines after treatment with LPS.

### Example 3

### Pro-Inflammatory Phenotype of S1R-Deficient Macrophages does not Require the Canonical Inflammatory Signaling Pathways

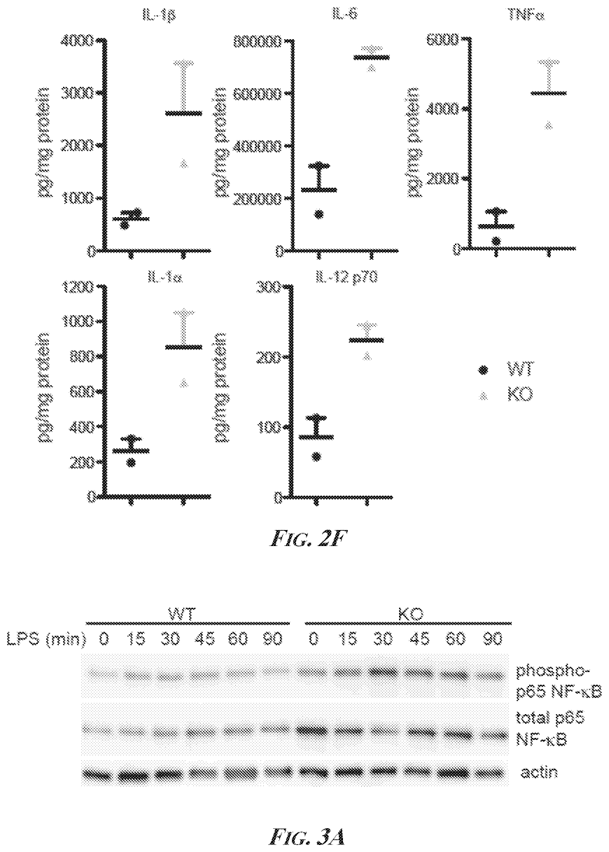

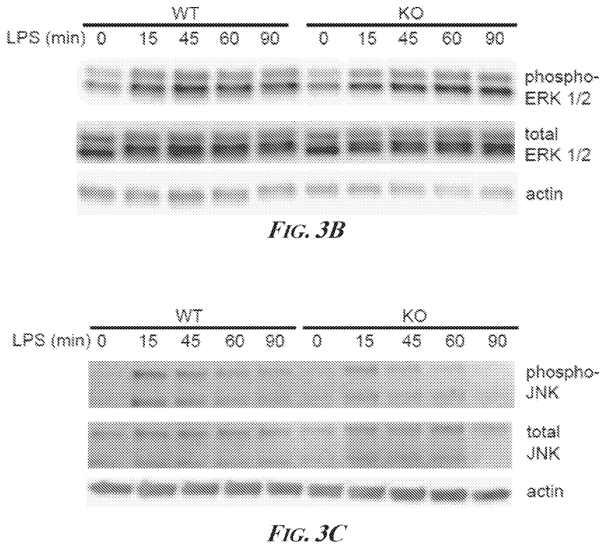

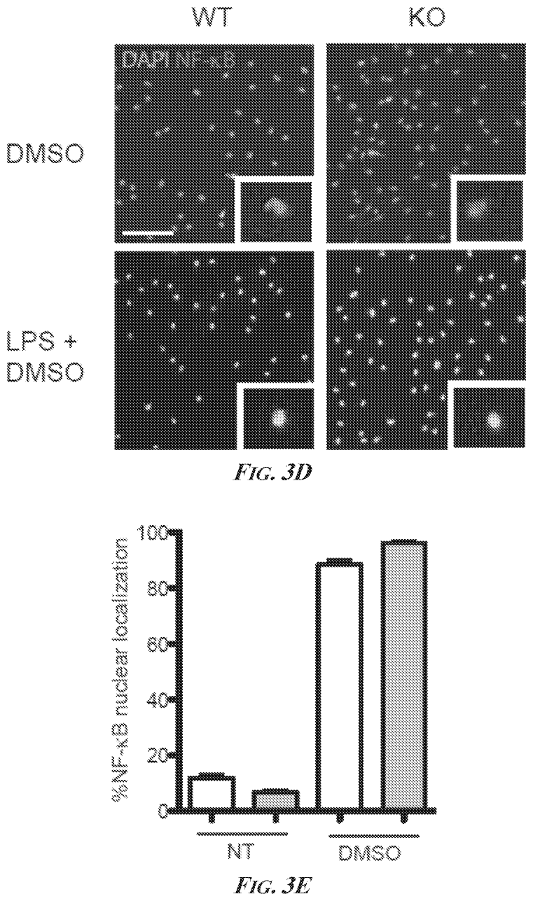

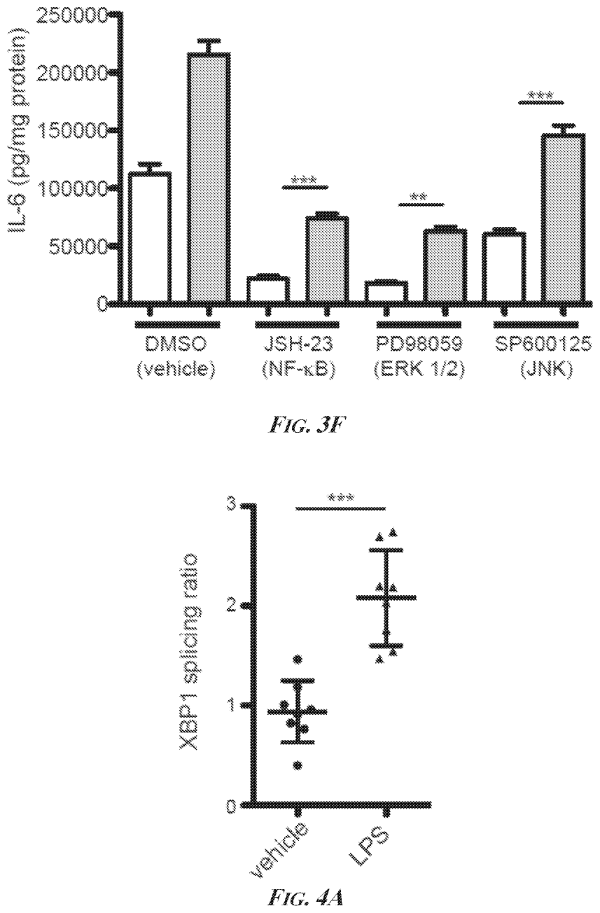

We next examined the activation status of proteins known to participate in the inflammatory response to LPS. A central signaling event in the induction of pro-inflammatory cytokine expression is the phosphorylation of NF-κB, a transcriptional master regulator of inflammatory cytokine expression (Lawrence, 2009). We examined whether NF-κB activation was different in S1R KO BMDM when compared to control cells, by monitoring phosphorylation of the p65 NF-κB subunit. Under the basal culture conditions, we found that phosphor-p65 NF-κB levels are higher in S1R KO than in control cells, potentially indicating a more inflammation-prone basal state (FIG. 3A). As expected, phosphorylation of p65 NF-κB is rapidly induced after LPS administration in both cell types; however, LPS-induced phosphorylation of p65 is also potentiated in S1R KO cells in comparison to LPS stimulated WT BMDM (FIG. 3A). Surprisingly, total p65 NF-κB protein levels were also elevated in S1R KO BMDM when compared to control, suggesting that greater p65 phosphorylation in S1R KO cells may be the result of increased protein abundance, instead of potentiated kinase activity. Importantly, the phosphorylation profiles of mitogen activated protein kinase ERK1/2 (FIG. 3B) and the c-Jun N-terminal kinase, JNK (FIG. 3C), were not affected by the absence of S1R, indicating that S1R expression preferentially affects the NF-κB signaling pathway.

NF-κB transcriptional activity requires the translocation of p65 from the cytosol into the nucleus (Vallabhapurapu & Karin, 2009). To determine if the observed increase in the amount of phosphor-p65 NF-κB correlated with an increase in its nuclear localization, in S1R knockout BMDM, we analyzed nuclear translocation of p65 NF-κB by immunofluorescence in LPS stimulated WT and S1R KO cells (FIGS. 3D and 3E). Remarkably, the percentage of cells displaying nuclear localization of p65 NF-κB was not significantly different between the WT and S1R KO cells under either the basal or the LPS stimulated state, suggesting that S1R does not alter the propensity of NF-κB to enter the nucleus after stimulation.

To further test the hypothesis that cytosolic signaling proteins like NF-κB do not drive increased cytokine secretion in S1R KO BMDM, we addressed whether chemical inhibition of NF-κB activity can eliminate the difference in pro-inflammatory cytokine expression between WT and S1R KO cells. We treated WT and S1R KO BMDM with JSH-23, a specific inhibitor of NF-κB (Shin et al., 2004), and monitored IL-6 protein secretion by ELISA. Treatment with JSH-23 reduced LPS-induced IL-6 secretion in comparison to vehicle-treated cells of both genotypes (FIG. 3F). However, JSH-23 did not eliminate the difference between the WT and S1R KO cells, with S1R KO BMDM secreting higher amounts of IL-6 even in the presence of this NF-κB inhibitor. Similarly, chemical inhibition of ERK1/2 (PRESENTLY DISCLOSED 98059) and JNK (SP600125) pathways also resulted in a significant decrease in IL-6 production in both genotypes without altering the ability of S1R KO BMDM to produce more IL-6, when compared to WT cells (FIG. 3F). Taken together, our results suggest that, while S1R acts a robust inhibitor of inflammation, it does not regulate the inflammatory response to LPS via the canonical inflammatory signaling pathways: NF-κB, ERK1/2, and JNK.

### Example 4

### S1R Controls the Endonuclease Activity of IRE1 During Inflammation

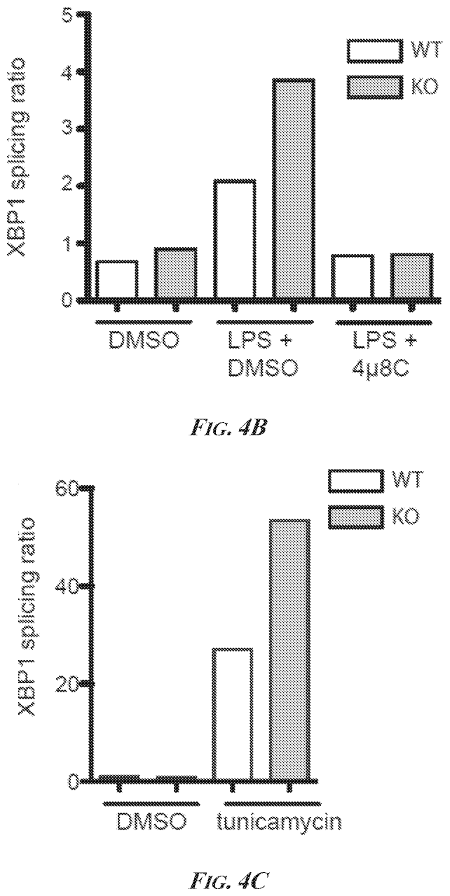

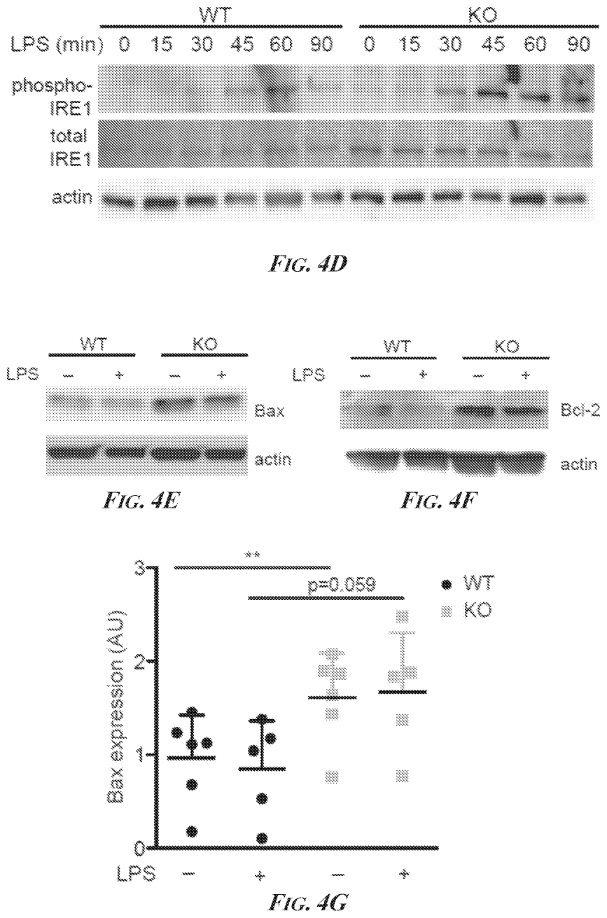

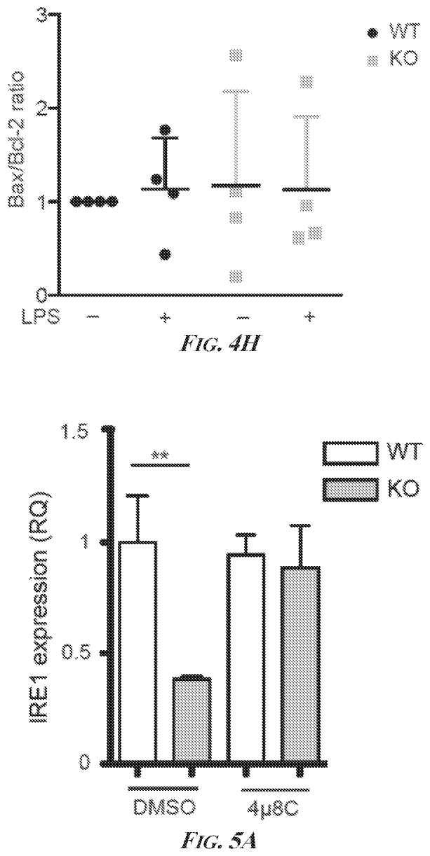

LPS stimulation has been shown to lead to activation of IRE1, an ER resident signaling protein, through redox dysregulation (Martinon et al., 2010). Upon activation, IRE1 gains the ability to cleave mRNAs, leading to their degradation. Endonuclease activity of IRE1 also leads to the expression of the transcription factor XBP1 (Chen & Brandizzi, 2013). In the context of inflammation, XBP1 regulates expression of numerous pro-inflammatory cytokines, including IL-6 and TNF-α (Martinon et al., 2010). Therefore, we next asked if S1R could inhibit LPS induced inflammation via regulation of IRE1 signaling. We began by examining IRE1 endonuclease activity in vivo during endotoxin challenge, by monitoring XBP1 splicing. XBP1 splicing is significantly elevated in peritoneal cells 3 hours after LPS administration (FIG. 4A). Next, we examined LPS-induced splicing of XBP1 mRNA in S1R KO BMDM. Exposure to LPS causes splicing of XBP1 in both WT and S1R KO BMDM, but the induction of XBP1 splicing is greater in BMDM lacking S1R, indicating that IRE1 endonuclease activity is more strongly induced in S1R KO BMDM after exposure to LPS (FIG. 4B). To confirm that XBP1 splicing was mediated by IRE1 endonuclease activity, the selective IRE1 endonuclease inhibitor 4μ8C was co-administered with LPS (Cross et al., 2012). Treatment with 4μ8C completely abolished LPS induced XBP1 splicing in both cell types. The enhanced XBP1 splicing in S1R KO BMDM is not specific to LPS exposure, as treatment with tunicamycin, which disrupts protein secretion and strongly induces ER signaling, also results in elevated XBP1 splicing in S1R KO BMDM (FIG. 4C). We next examined IRE1 phosphorylation in BMDM stimulated with LPS. We observed rapid phosphorylation of IRE1 in response to LPS in both the WT and S1R KO cells, with increased IRE1 phosphorylation in S1R KO BMDM (FIG. 4D). Intriguingly, we also noted that the total amount of IRE1 in S1R KO BMDM was elevated in comparison to WT BMDM, suggesting that S1R may control the IRE1 protein levels (FIG. 4D).

Because S1R has been reported to alter the abundance of the pro-apoptotic protein Bax (Tchedre & Yorio, 2008), which has, in turn, been shown to potentiate IRE1 endonuclease activity in an apoptosis-independent manner (Hetz et al., 2006), we next examined Bax levels in the S1R KO BMDM by western blot analysis (FIGS. 4E and 4G). Indeed, we observed significantly elevated Bax levels in S1R KO cells, which were not altered by LPS stimulation during the course of the treatment. Surprisingly, the level of the anti-apoptotic protein Bcl-2 was also raised in S1R KO BMDM (FIGS. 4F and 4H), leading to a similar ratio Bax/Bcl-2, suggesting that the elevated Bax may drive IRE1 activity without affecting apoptosis, as previously described (Hetz et al., 2006).

### Example 5

### S1R Regulates IRE1 Protein Abundance Post-Translationally

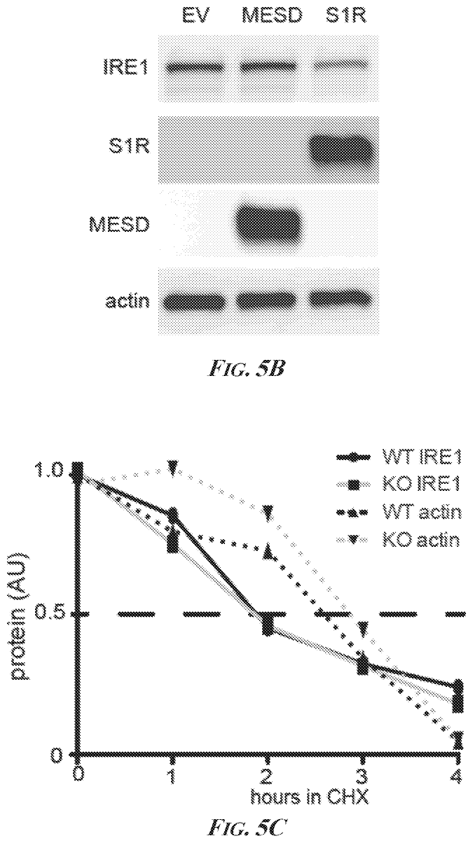

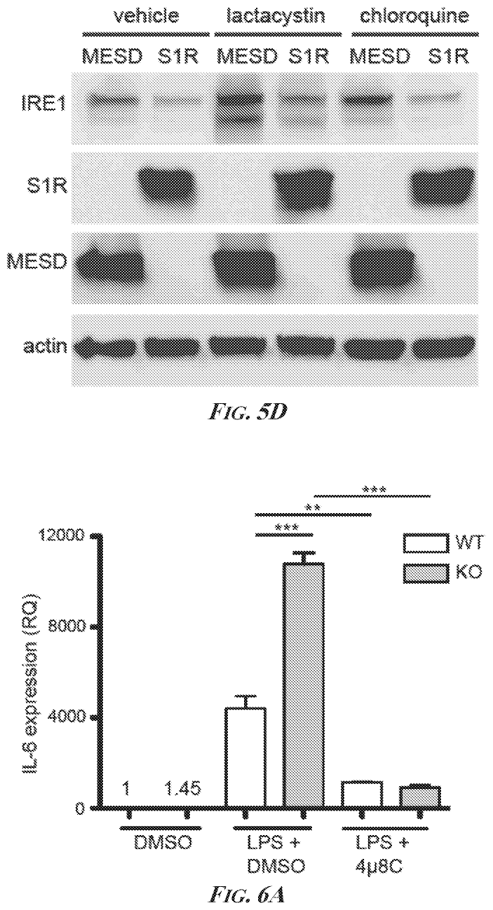

Since we observed an increase in IRE1 protein levels in S1R KO cells, we examined the mechanisms by which S1R might influence IRE1 abundance. First, we measured transcription of IRE1 by qPCR. Remarkably, increased IRE1 protein levels in S1R KO BMDM were not due to an increase in the IRE1 transcript levels. On the contrary, qPCR analysis revealed that the IRE1 mRNA levels were actually lower in S1R KO BMDM (FIG. 5A). However, this surprising result is in agreement with the study by Tirasophon et al. that demonstrated that the IRE1 protein promotes degradation of its own transcript, in a manner directly mediated by the IRE1 endonuclease activity (Tirasophon et al., 2000). Indeed, inhibition of IRE1 endonuclease activity by 4μ8C restores IRE1 transcript levels to those observed in WT BMDM (FIG. 5A). We next tested if S1R over-expression can decrease IRE1 expression, by co-transfecting IRE1 and S1R in HEK293 cells. Overexpression of S1R results in a significant decrease in IRE1 abundance relative to the empty vector control (FIG. 5B).

To ensure that our results were specific for S1R and not the result of non-specific overexpression of an ER resident protein, we also co-expressed IRE1 with another ER-resident protein, MESD (Hsieh et al., 2003). Overexpression of MESD did not alter the levels of IRE1 (FIG. 5B), suggesting that increased S1R expression specifically decreases IRE1, and inversely, S1R deficiency leads to an increase in IRE1. Since S1R does not alter IRE1 expression at the transcript level, we examined whether S1R regulates IRE1 protein degradation. We evaluated the half-life of the IRE1 protein in WT and S1R KO BMDM that were treated with the translation inhibitor cycloheximide. We found that IRE1 protein half-life is not increased in S1R KO BMDM in comparison to control cells, suggesting that S1R does not regulate IRE1 degradation (FIG. 5C).

Finally, we tested whether the S1R-mediated decrease in IRE1 abundance in HEK293 cells can be rescued by treating the cells with lactacystin or chloroquine, which specifically block the proteasomal or lysosomal degradation pathways (Blok et al., 1981; Fenteany & Schreiber, 1998). Neither lactacystin nor chloroquine treatment could rescue IRE1 protein levels in the presence of S1R overexpression (FIG. 5D). Proteasome inhibition leads to an overall increase in IRE1 quantity independently of the transfection conditions, consistent with the reports of IRE1 degradation by the proteasome (Gao et al., 2008). Taken together, our results demonstrate that S1R influences IRE1 protein levels without directly altering IRE1 transcription or protein degradation.

### Example 6

### Unshackled IRE1 Endonuclease Drives Inflammatory Cytokine Expression in S1R KO BMDM

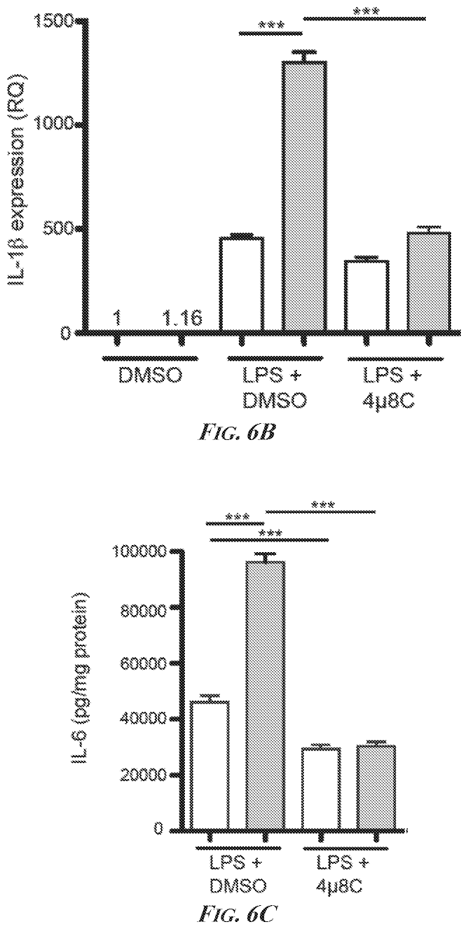

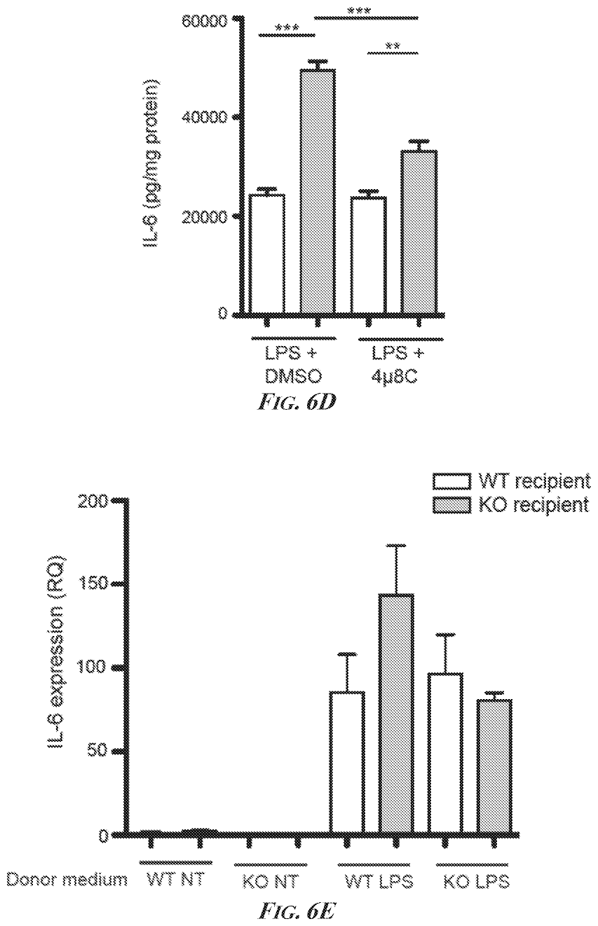

To further test if IRE1 endonuclease activity is directly responsible for the increase in pro-inflammatory cytokine expression, we treated WT and S1R KO BMDM with LPS and 4μ8C and analyzed cytokine expression by qPCR. Inhibition of IRE1 endonuclease activity decreases IL-6 and IL-1β expression in both genotypes and inhibits potentiation of cytokine expression in S1R KO BMDM (FIGS. 6A and 6B). We also examined IL-6 secretion by ELISA. In agreement with the transcript results, 4μ8C rescued IL-6 secretion in S1R KO cells to the levels observed in control BMDM (FIG. 6C). Furthermore, the effect of S1R on inflammatory mediator production does not appear to be limited to macrophages, as S1R KO lung fibroblasts secrete 2-fold higher levels of IL-6 upon LPS stimulation, and the concomitant treatment with 4μ8C also results in a marked decrease of cytokine secretion (FIG. 6D). These data suggest that the S1R dependent inhibition of LPS-inducible cytokines is the result of restraint of the endonuclease activity of the ER stress sensor IRE1. Considering the elevated secretion of inflammatory cytokines by S1R KO BMDM, it is possible that increased inflammatory signaling observed in S1R KO BMDM might be due to the abundance of cytokines like IL-6 and IL-1β, instead of a primary response to LPS itself. To address this, we collected the supernatants of WT or S1R KO BMDM that were cultured under control or LPS stimulated conditions, and used those supernatants to treat WT and S1R KO BMDM. If elevated inflammatory cytokines were driving inflammatory transcription during the in vitro LPS challenge in a feed-forward manner, we would expect to see that the supernatants conditioned by S1R KO BMDM would induce more inflammatory transcripts in the recipients of either genotype. However, transcription of IL-6 and IL-1β was consistent across recipients treated with LPS conditioned supernatants, regardless of the donor cell genotype (FIGS. 6E and 6F). These data indicate that elevated inflammatory cytokine production in S1R KO BMDM depends on the cell intrinsic signaling by LPS and is largely unaffected by the cytokine milieu.

### Example 7

### Inhibition of IRE1 Endonuclease Activity Rescues S1R KO Mice from Septic Mortality

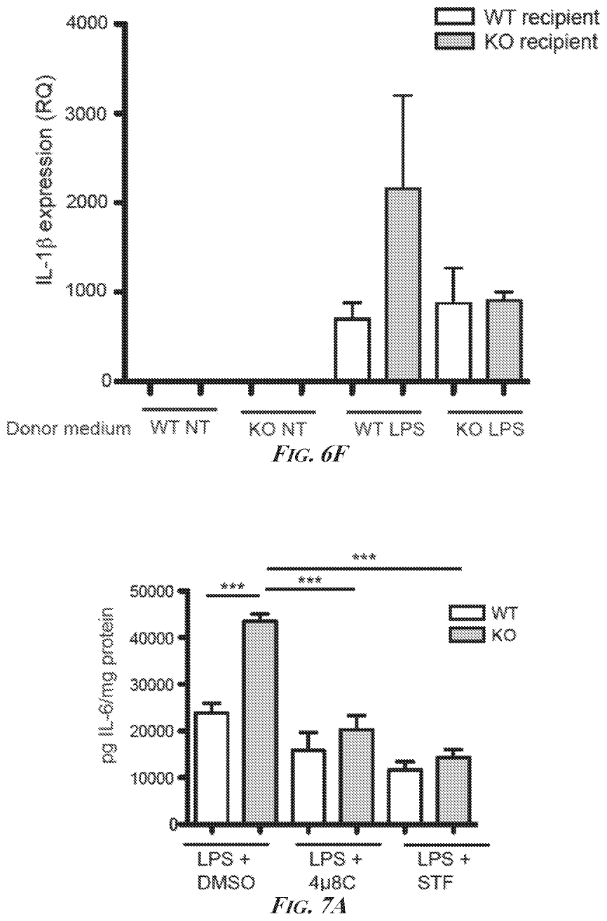

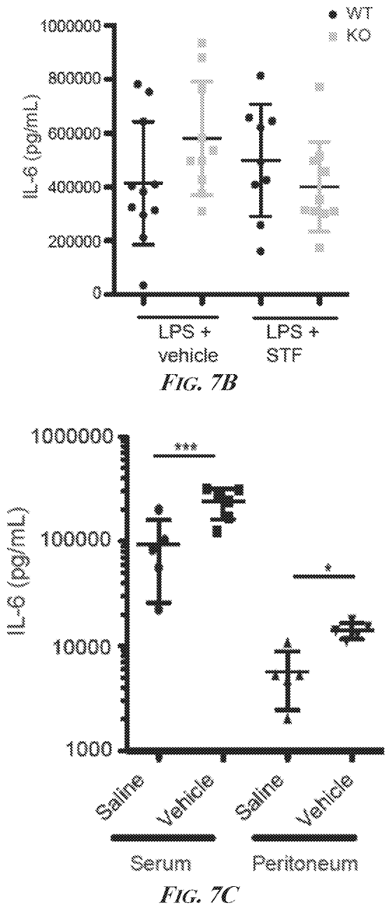

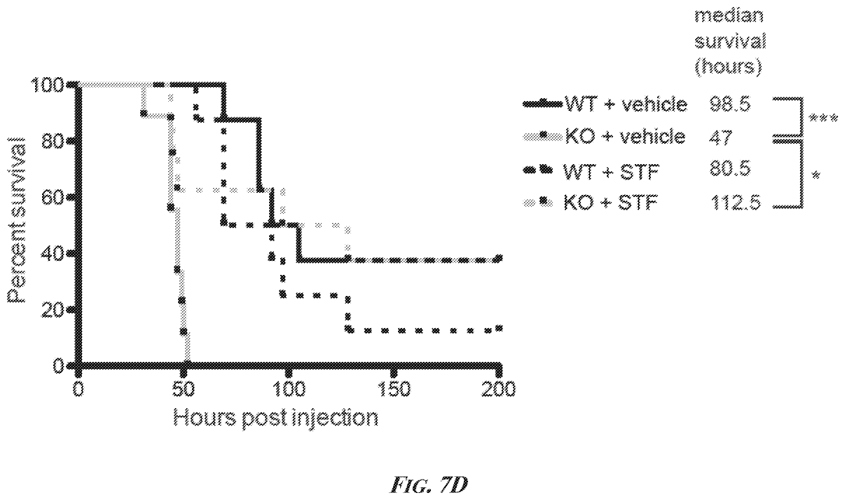

Because lack of S1R potently induces IRE1-dependent cytokine production in macrophages in vitro, and S1R KO mice are susceptible to a normally sub-lethal dose of LPS, we tested whether inhibiting IRE1 could improve the survival of S1R KO mice during the endotoxin challenge. Due to the reported short half-life of 4μ8C in vivo (Cross et al., 2012), we employed an alternative IRE1 inhibitor, STF 083010 (herein referred to as STF), which has been shown to inhibit IRE1 in a mouse model of multiple myeloma (Papandreou et al., 2011; Cross et al., 2012). In our hands, STF was as effective as 4μ8C in blocking IL-6 secretion in S1R KO BMDM stimulated with LPS (FIG. 7A). Next, we co-administered LPS and STF in WT and S1R KO mice and monitored the level of circulating IL-6 in the serum. Three hours post LPS administration, we observed a trend toward increased IL-6 levels in the serum of S1R KO mice compared to WT mice treated with the vehicle control for STF (33% Kolliphor-EL in saline). However, the difference between WT and S1R KO mice treated with LPS and the vehicle for STF did not reach statistical significance, in contrast to our results observed in mice treated with LPS alone (FIG. 1C). Importantly, treatment with STF significantly decreased serum IL-6 in KO mice (FIG. 7B). This may be due to the much higher levels of serum cytokine in mice treated with LPS and vehicle, as we observed that the vehicle treatment robustly potentiated LPS induced IL-6 levels in both the serum and the peritoneal cavity of WT mice (FIG. 7C).

Intraperitoneal injection of vehicle alone did not result in a detectable production of IL-6, nor did it cause any mortality in WT mice, suggesting that the vehicle treatment exacerbates LPS induced inflammation. Therefore, we chose to administer a slightly lower dose of LPS (2 mg/kg) for the in vivo challenge of WT and S1R KO mice, in order to examine whether IRE1 inhibition by STF could improve the survival of S1R KO mice. Paralleling our observation with LPS alone (see FIG. 1A), S1R KO mice that received LPS with the vehicle control for STF experienced rapid mortality (FIG. 7D). Remarkably, despite increased inflammation in mice that received LPS in combination with the vehicle, STF administration largely rescued KO mice from LPS induced mortality (FIG. 7D). Median survival was 47 hours for S1R KO versus 99 hours in WT mice that received vehicle. When LPS was co-administered with STF, S1R KO mice experienced a significantly lower mortality (62.5%), and a median survival of 113 hours, which was similar to the vehicle-treated WT animals. STF treatment did not significantly affect the survival of WT mice (FIG. 7D). We also detected a significantly higher level of IL-6 in the peritoneal exudate in vehicle-treated S1R KO mice compared to WT, which was corrected by STF treatment (FIG. 7E). These findings are in agreement with our hypothesis that cytokine production and endotoxin-induced mortality in S1R KO mice require excessive IRE1 endonuclease activity. Overall, administration of an IRE1 inhibitor (STF) can rescue the phenotype driven by S1R deficiency; development of optimized small molecules for IRE1 inhibition or S1R activation could therefore offer novel treatment options for sepsis in the future.

### Discussion of Examples 1-7

The ER stress sensing protein IRE1 is able to powerfully affect the inflammatory behavior of both immune and non-immune cells in numerous contexts (Cho et al., 2013; Janssens et al., 2014). However, little is yet known about the factors that modulate the extent of IRE1 signaling during inflammation. Here we identify S1R as a novel regulator of IRE1 endonuclease function in macrophages during LPS-induced inflammation. This finding is particularly exciting, since the physiological and pathophysiological relevance of IRE1 is well established. However, drugging IRE1 directly might produce intolerable side effects in humans, as IRE1 activity is essential for a range of normal biological functions, including insulin secretion and lipid maintenance in the liver (Lipson et al., 2006; Fu et al., 2012). Therefore, an alternative route towards affecting IRE1 signaling holds significant therapeutic promise. S1R, a ubiquitously expressed protein with affinity for numerous drugs already in clinical use represents one such alternative route (Hashimoto, 2015).

Here we show that, in the absence of S1R, unshackled IRE1 endonuclease activity leads to increased production of inflammatory mediators. Importantly, the relationship between S1R and IRE1 appears to be independent of inflammatory cytosolic signaling pathways such as NF-κB, ERK, and JNK. Furthermore, lack of S1R appears to selectively enhance activity of IRE1 and not other inflammatory pathways, as inhibition of NF-κB, JNK and ERK did not correct the pro-inflammatory phenotype associated with S1R KO BMDM. This interaction will be important to study, as there are a multitude of inflammatory and degenerative diseases in which S1R and IRE1 dysfunction are potentially implicated, including Alzheimer's disease (Marrazzo et al., 2005; Salminen et al., 2009) and amyotrophic lateral sclerosis (Kikuchi et al., 2006; Mavlyutov et al., 2015). It will be critical to continue to elucidate the full range of situations in which the S1R-IRE1 interaction impacts pathology.

Consistent with previous findings indicating the importance of IRE1 during inflammatory signaling (Martinon et al., 2010), we observe rapid phosphorylation of IRE1 in response to LPS exposure in BMDM. Although S1R has previously been identified as a factor that decreases the rate of IRE1 phosphorylation and degradation in cells exposed to thapsigargin (Hayashi & Su, 2007), we did not observe an S1R-dependent difference in the kinetics of IRE1 phosphorylation of LPS-treated macrophages, perhaps reflecting different modes of IRE1 activation downstream of the two stimuli. In our hands, S1R KO is likely affecting inflammation by Bax dependent increase in IRE1 endonuclease activity (Hetz et al., 2006). Furthermore, S1R seems to control IRE1 protein levels, as overexpression of S1R leads to a significant decrease in IRE1 abundance and, conversely, S1R deficiency correlates with increased IRE1. We propose that the variation in IRE1 quantity can directly affect IRE1 endonuclease activity. Interestingly, the effect of S1R on IRE1 abundance did not occur at the transcriptional level. Indeed, in S1R KO BMDM we could detect a lower amount of the IRE1 transcript, despite protein abundance elevation when compared to WT cells. This discrepancy may be explained by the fact that IRE1 protein degrades its autologous mRNA (Tirasophon et al., 2000). We have also demonstrated that S1R does not affect the IRE1 protein stability, as the protein half-life was similar in WT and S1R KO BMDM, and inhibition of the proteasome or lysosome functions did not correct the difference in IRE1 levels. Taken together, our data suggest that S1R is a novel translational regulator for IRE1. Further experiments will be needed to understand the mechanism by which S1R controls the translation of IRE1.

Previous studies have suggested a possible point of intersection between S1R and IRE1 signaling pathways via Bcl-2 family proteins. Knockdown of S1R has been shown to decrease levels of Bcl-2, an anti-apoptotic protein that can inhibit Bax (38). Bax, in turn, potentiates IRE1 endonuclease activity (Hetz et al., 2006). We have shown here that S1R deficiency raises both Bax and Bcl-2 levels in BMDM. Therefore, in addition to its influence on IRE1 abundance, S1R KO may potentiate IRE1 by increasing the amount of Bax that is available for IRE1 activation.

XBP1 is recognized as a critical factor in the cellular response to LPS-induced inflammation (Martinon et al., 2010). Although we did not pursue this line of inquiry, inhibition of IRE1 endonuclease activity also decreased inflammatory activity in the WT condition in nearly every inflammatory context we examined, both in vitro and in vivo. It is possible that inflammatory challenge and IRE1 inhibitory treatment paradigms could be optimized to show the full extent of the effect that IRE1 inhibition might have on inflammatory functions in WT macrophages and mice in vivo. Pursuit of this exciting prospect is likely to yield further instances in which modulation of IRE1, whether through S1R or otherwise, may be beneficial to human health.

In this work, we identify S1R as a critical regulator of IRE1 driven inflammation. S1R deficiency potently enhances inflammatory cytokine production in a manner dependent on IRE1 activity and reduces survival during models of hyper-inflammation and septic shock in mice. Conversely, forced expression of S1R can dampen the inflammatory response to LPS. Further, we show that the S1R ligand fluvoxamine can enhance survival in mouse models of sepsis and can inhibit the inflammatory response in human peripheral blood cells. Collectively, our data show that S1R is uniquely poised to sensitively control IRE1 activity during inflammation.

### Materials and Methods for Examples 8-12

Study design. The goal of our study was to identify the role of S1R during LPS mediated inflammation. Using animal models of sepsis, we demonstrated the fact that S1R is an inhibitor of cytokine production. We elucidated the mechanism by which S1R controls the inflammatory response via IRE1 with primary BMDM and HEK293. Using pharmacological inhibitors, we used two in vivo models of sepsis to validate our in vitro findings showing the mechanism of action of S1R and IRE1. We finally confirm our results obtained with fluvoxamine using human blood samples. In all experiments, animals were randomly assigned to treatment groups and researchers were blinded during treatment and data collection. Group and sample size for each experiment are indicated in each figure legend. No statistical methods were used to predetermine sample sizes for in vitro experiments. Sample sizes for in vivo and ex vivo experiments were predetermined using G*Power, with 1−β≥0.85. Post-hoc power calculations were performed on in vitro studies (except where representative data is shown) using G*Power to ensure that 1−β≥0.80.

Mice. C57BL/6J (8 weeks old) were purchased from Jackson. The S1R knockout mouse strain was acquired from MMRRC and bred to C57BL/6J at the University of Virginia to generate mice used in the study (Sabino et al., 2009; Ha et al., 2011). All animal experiments were approved and complied with regulations of the Institutional Animal Care and Use Committee at University of Virginia.

Tissue culture conditions and Reagents. HEK293 mTLR4/MD2/CD14 (Invivogen, 293-mtlr4md2cd14), primary lung fibroblasts, and BMDM were isolated and maintained as described (Zhang et al., 2008; Seluanov et al., 2010). Cells and animals were treated with LPS (Sigma, L4391), 4μ8C (Tocris, 4479), APY-29 (Medchem Express HY-17537), PD98059 (Medchem Express HY-12028), JSH-23 (Medchem Express, HY-13982), SP600125 (Medchem Express, HY-12041), STF 083010 (Medchem Express, HY-15845), Fluvoxamine (Medchem Express, HY-B0103A), and Ceftriaxone (Hospira, NDC: 0409-7337-01), as described in the text.

LPS challenge. In vivo LPS challenge was performed on adult mice (8-12 weeks of age). LPS from E. coli 0111:B4 (Sigma-Aldrich, L2630) was injected intraperitoneally, as described in the manuscript. STF083010 (Medchem Express, HY-15845) was resuspended in 33% Kolliphor-EL (Sigma, C5135) and administered intraperitoneally at 30 mg/kg immediately after and again 24 hours after LPS injection. Fluvoxamine was resuspended in saline and administered at 20 mg/kg as indicated in the text. Blood for serum ELISA was collected from facial vein at the peak serum concentration of TNF-α and IL-6 (Copeland et al., 2005a).

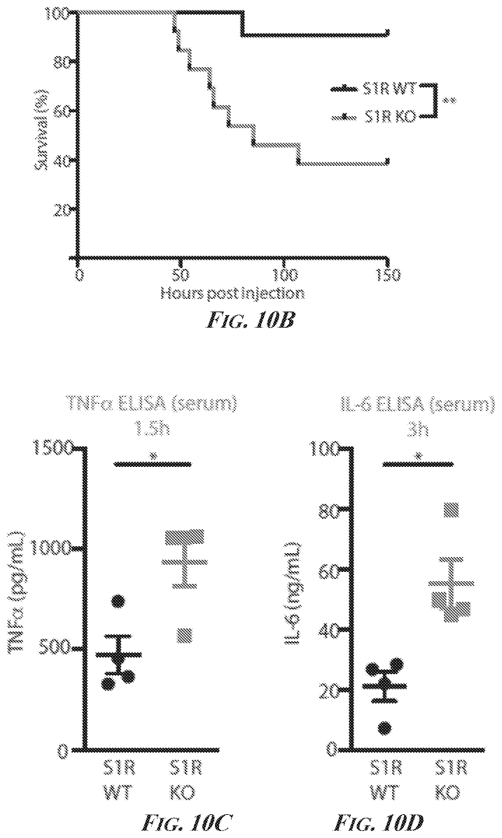

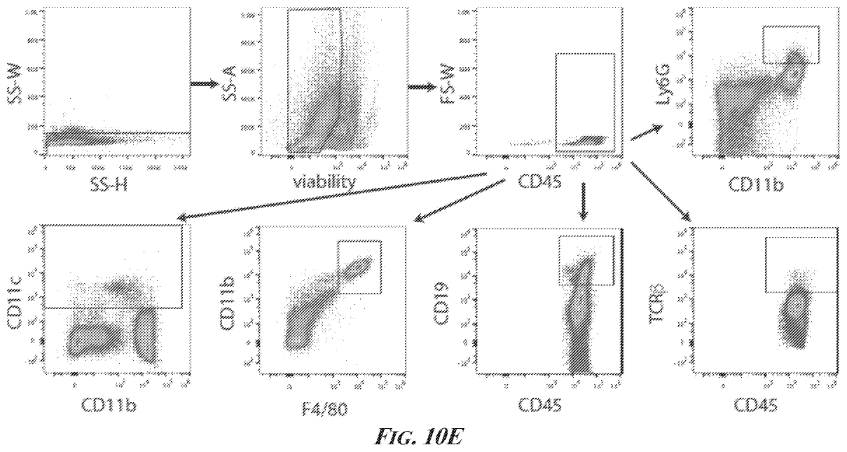

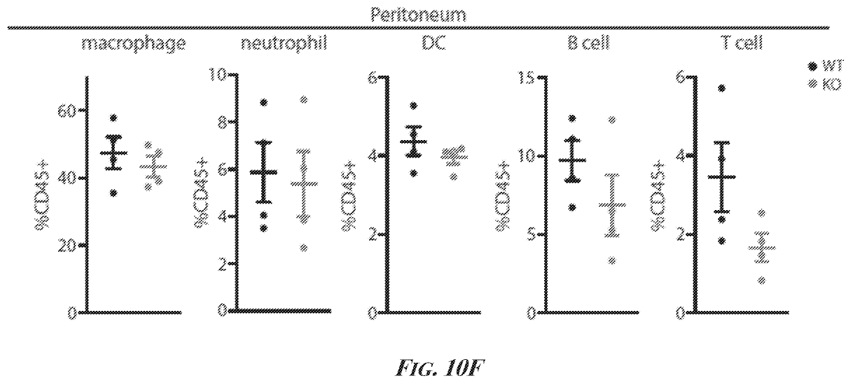

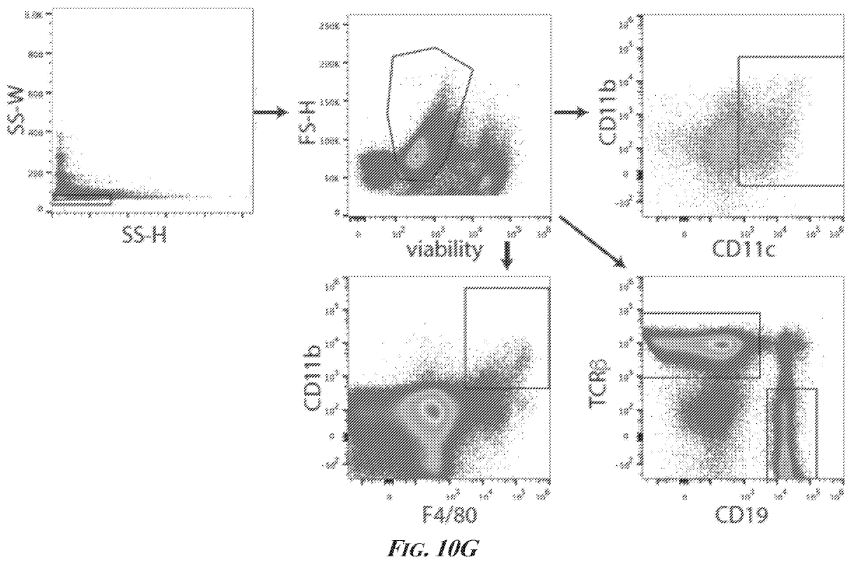

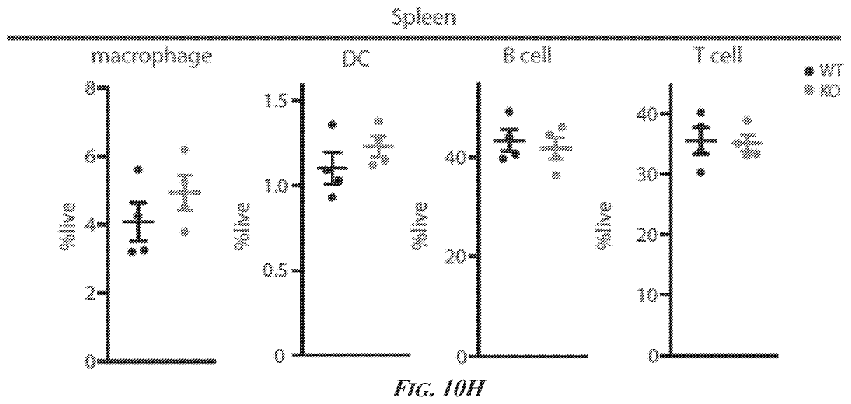

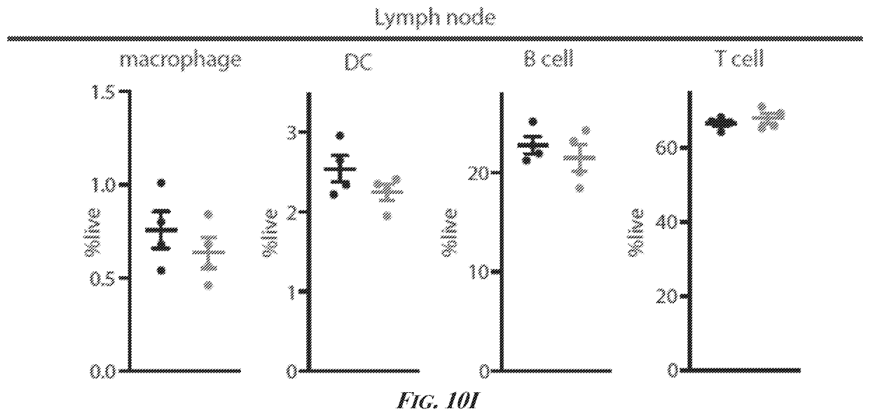

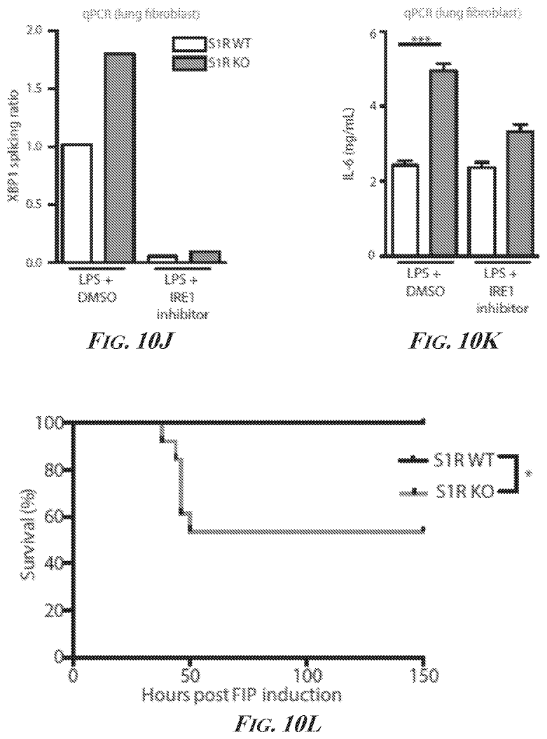

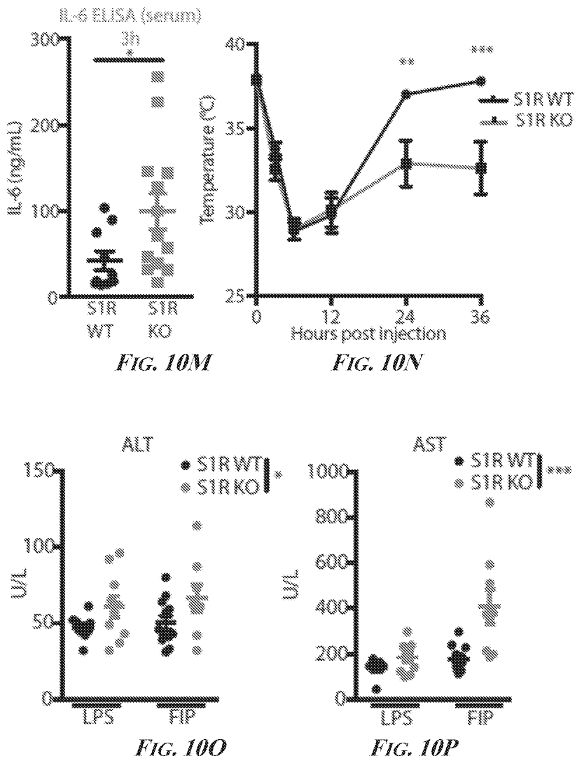

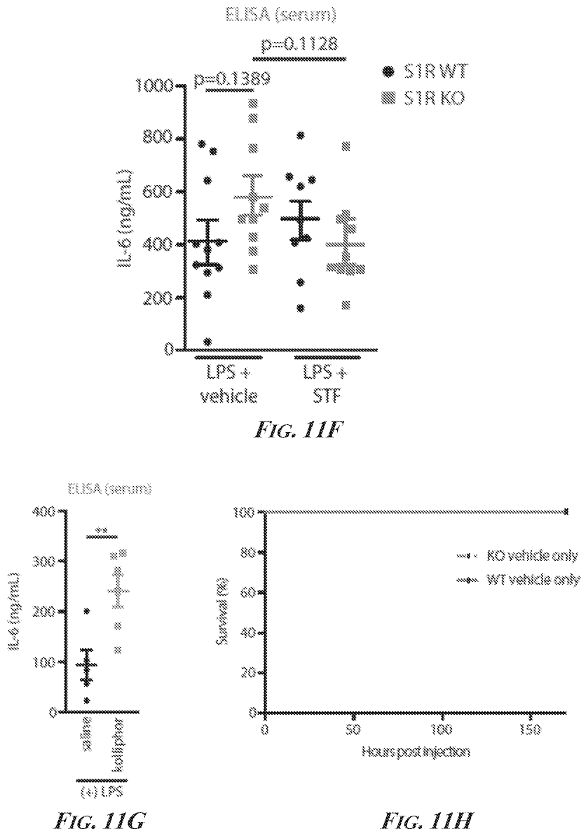

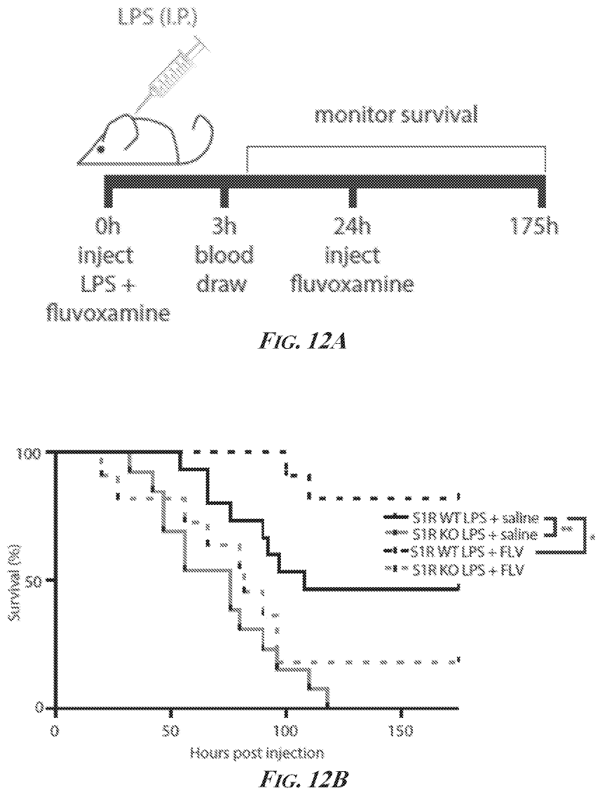

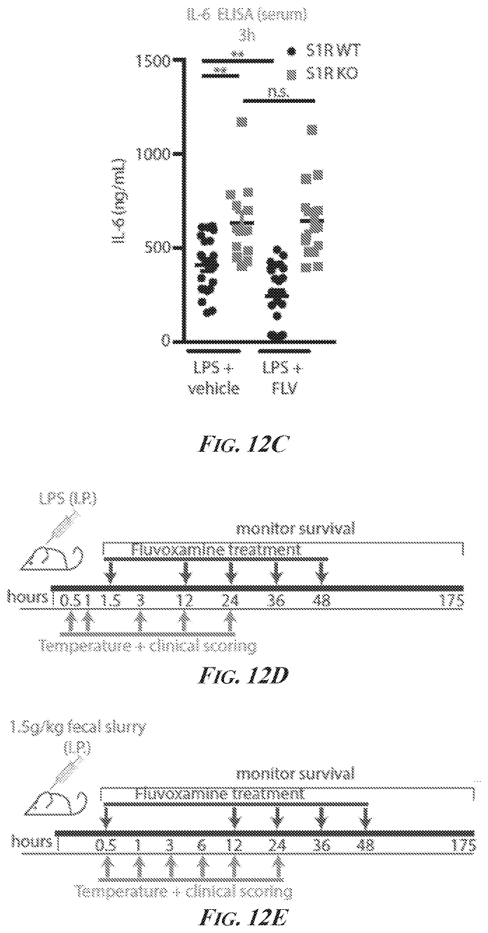

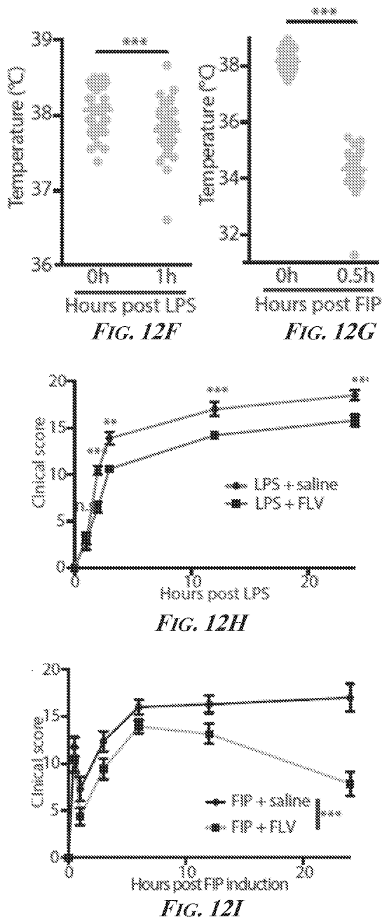

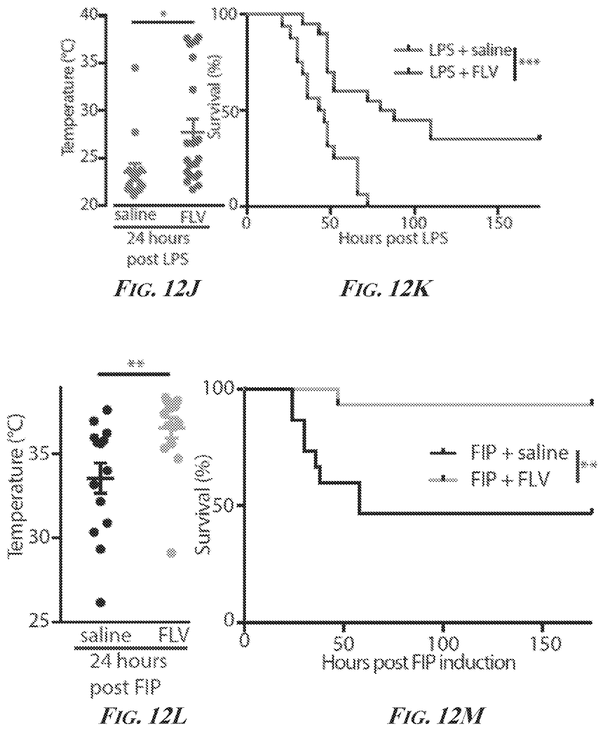

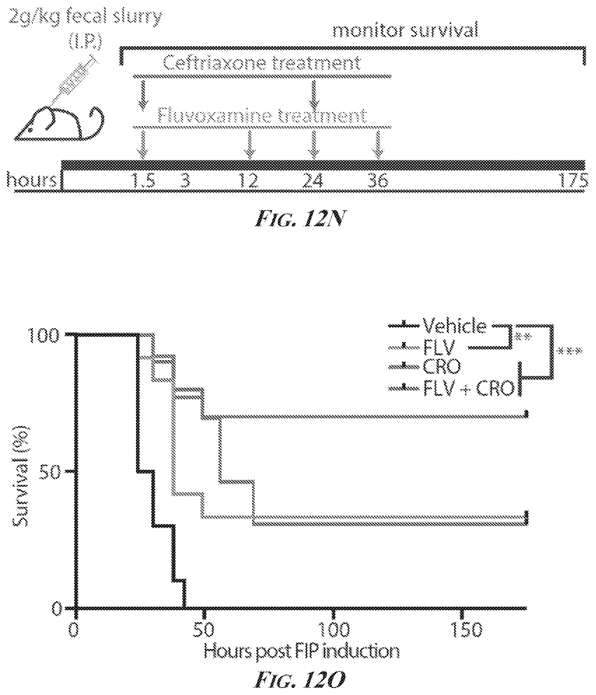

Fecal induced peritonitis (FIP). Fecal material was isolated from the caecum of age- and sex-matched WT animals coming from the UVA vivarium for FIGS. 10A-10D and 10L-10R or from Jackson Laboratory for FIGS. 12D-12O, resuspended in saline and passed through a 70 μM strainer to remove large particles. The slurry was prepared fresh for each experiment. Core body temperature was measured and mice were scored with murine sepsis severity scale by two independent, blinded researchers (Shrum et al., 2014). Blood for serum ELISA was collected from facial vein at 3 hours post FIP induction. Where indicated, fluvoxamine in saline was administered intraperitoneally at a dose of 20 mg/kg, and ceftriaxone in saline was given at a dose of 100 mg/kg subcutaneously.

Serum preparation. Serum was collected 24 hours after injection of LPS or fecal slurry. Serum chemistry analysis was performed by Comparative Clinical Pathology Services LLC. ELISA was performed on serum as described below.

ELISA. ELISA for IL-6 and TNF-α were performed as previously described (Remick et al., 2002). Antibodies used were: anti-mouse IL-6 MP5-20F3 (Biolegend, 504501) 0.5 μg/mL; biotin anti-mouse IL-6 MP5-32C11 (Biolegend, 504601) 1 μg/mL; anti-mouse TNF-α (R&D systems, AF-410-NA) 0.5 μg/mL; biotin anti-mouse TNF-α (R&D systems, BAF410) 0.25 μg/mL.

Peritoneal exudates collection. Peritoneal cavities content were collected 3 hours after LPS injection in PBS+5 mM EDTA, then centrifuged to pellet cells. Supernatants were collected for ELISA, and cells were washed and stored as previously described (Gaultier et al., 2008).

Western blot. Protein extraction and western blot were performed as previously described (Gaultier et al., 2008). Antibodies were used according to manufacturer's instruction: Actin (Sigma Aldrich, A2228) 1:5000; BiP (BD Biosciences, 610798) 1:1000); total ERK1/2 (CST, 9102) 1:1000; phospho-ERK1/2 (CST, 4370) 1:1000; total IRE1α (CST, 3294) 1:1000; 1:1000; total JNK (CST, 9252) 1:1000; phospho-JNK (CST, 9251) 1:1000; total p65 NF-κB (CST, 8242) 1:1000; phospho p65 NF-κB (CST, 3033) 1:1000; PDI (Abcam, 2792) 1:1000; total PERK (CST, 3192) 1:1000. Linear level adjustments were applied to entire images to enhance visualization.

Cloning and transfection. Plasmids used were: MGC Mouse Sigmar1 cDNA (GE Life Sciences, MMM1013-202768624) and pcDNA3.1 MCS-BirA(R118G) HA (addgene #36047) (Roux et al., 2012). S1R-BirA HA construct was generated by cloning Murine S1R ORF upstream of BiRA into pcDNA3.1 MCS-BirA HA. HEK293 mTLR4/MD2/CD14 were transfected using X-tremeGENE HP transfection reagent (Roche, 06366244001) according to the manufacturer's instructions.

Proximity biotinylation. Culture medium was supplemented with 80 μM biotin (Research Products International, B40040) and LPS 18 hours after transfection. Biotinylated protein were purified as described (Gaultier et al., 2008).

cDNA synthesis and quantitative PCR. Total RNA was extracted using an ISOLATE II RNA kit (Bioline, 52073) and cDNA synthesis was performed with the SENSIFAST™ cDNA synthesis kit (Bioline, BIO-65054). TaqMan Probes were obtained from Thermo Fisher (GAPDH: Mm99999915_g1; IL-6: Mm00446190_m1; pro-IL-1β: Mm00434228_m1; IL10: Mm004396). Primers for the detection of XBP1, IL-8, and actin were previously published (Vandesompele et al., 2002; Kumar & Valdivia, 2008; Girard et al., 2009). qPCR was performed as described previously (Gaultier et al., 2008).

Flow cytometry. Flow cytometric analyses were performed as described (Seki et al., 2017). The following Abs were used: TLR4/MD-2 Complex APC (Thermo Fisher Scientific, 17-9924-82), F4/80 Antigen PE (Thermo Fisher Scientific, 12-4801-80), F4/80 Antigen PE Cy7 (Thermo Fisher Scientific, 25-4801-82), CD11b PE Cy7 (Biolegend, 101215), CD11b eFluor 450 (Thermo Fisher Scientific, 48-0112-82), CD11b APC (Thermo Fisher Scientific, 17-0112-82), CD45 PerCP Cy5.5 (Thermo Fisher Scientific, 45-0451-82), CD45 APC (Thermo Fisher Scientific, 17-0451-82), Ly6G APC Cy7 (Tonbo Biosciences, 25-1276), CD19 PE Cy7 (Thermo Fisher Scientific, 25-0193-82), CD4 eFluor 450 (Thermo Fisher Scientific, 48-0042-82), TCRβ (Thermo Fisher Scientific, 12-5961-83), CD8 Alexa Fluor 488 (Thermo Fisher Scientific, 53-0081-82), CD11c PerCP Cy5.5(Thermo Fisher Scientific, 45-0114-82), CD115 APC (Thermo Fisher Scientific, 17-1152-82), Fc Block (Thermo Fisher Scientific, 14-9161-71) and a Zombie Aqua Fixable Viability kit (Biolegend, 423101).

Human whole blood stimulation. Study participants were healthy adults (ages 18-45). The study was approved by the Institutional Review Board at the University of Virginia, and all participants signed informed consent before enrollment. Blood was collected into heparinized vacuum tube, then stimulated with 10 ng/mL LPS+/−20 μM fluvoxamine for 4 hours, as described (Thurm & Halsey, 2005). Cytokine concentrations were determined by multiplex analysis.

Data analysis and statistics. Data are represented as mean+/−SEM. Densitometry was performed using ImageJ software. Statistical analyses, as indicated in each figure legend, were performed using GraphPad Prism 6. All t-tests were two-tailed. ROUT analysis was used to identify outliers, with Q=1%, and outliers identified by this method were excluded from analysis. The D'Agostino & Pearson omnibus normality test was used to assess normality of data sets. Power analyses were performed with G*Power 3.1.

### Example 8

### S1R Controls LPS Induced IRE1 Activity in Macrophages

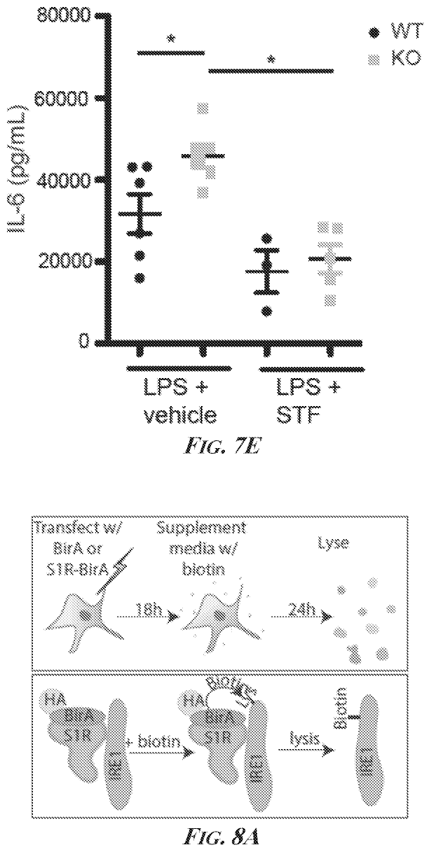

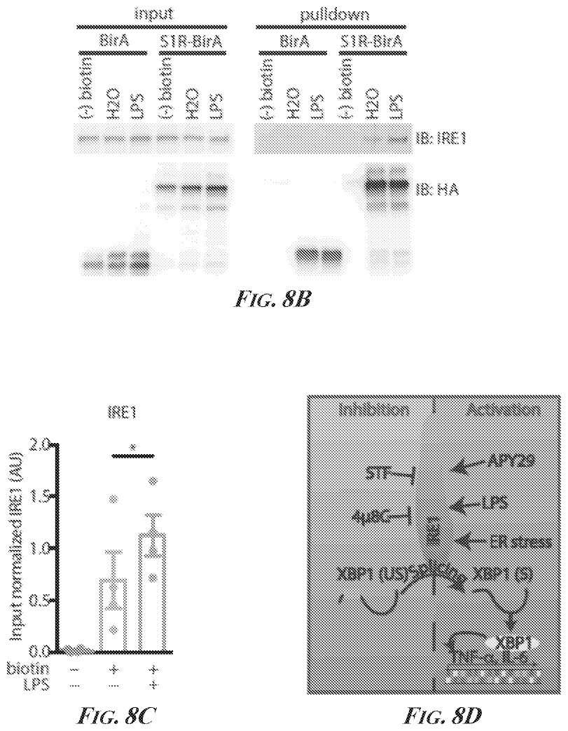

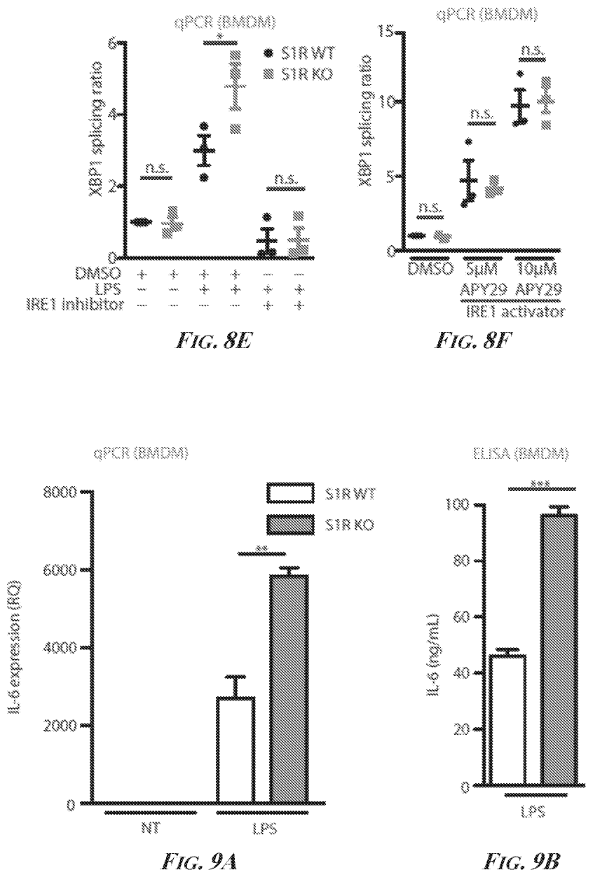

S1R was recently shown to interact with IRE1 under strong ER stress inducing conditions (Mori et al., 2013). Given the newly discovered role for IRE1 during the inflammatory response (Martinon et al., 2010; Qiu et al., 2013), we wanted to test if S1R participates in ER mediated inflammation. We first used the BirA proximity ligation assay to test if S1R interacts with IRE1 during LPS challenge (Roux et al., 2012). For this experiment, we used HEK293 cells that express mTLR4/MD2/CD14 and thereby respond to LPS (Pozzobon et al., 2016). Cells were transfected with S1R conjugated to the bifunctional ligase/repressor BirA (BirA), or BirA alone as control, resulting in the biotinylation of proteins that are in close proximity to S1R (FIG. 8A; Roux et al., 2012). We observed IRE1 biotinylation during homeostasis that was enhanced following LPS treatment (FIGS. 8B and 8C), indicating proximity and possible association (direct or indirect) between S1R and IRE1.

Upon activation with LPS, IRE1 endonuclease activity is triggered and splices the mRNA that encodes the transcription factor X-box binding protein-1 (XBP1; FIG. 8D), resulting in expression of active XBP1 protein. We found increased LPS-induced XBP1 splicing in bone marrow derived macrophages (BMDM) lacking S1R, indicating elevated inducible, but not basal, IRE1 endonuclease activity in S1R knockout (KO) macrophages (FIG. 8E). To confirm that XBP1 splicing was mediated by IRE1 endonuclease activity, the selective IRE1 endonuclease inhibitor 4μ8C was tested (FIG. 8E; Cross et al., 2012). Treatment with 4μ8C abolished LPS induced XBP1 splicing in both genotypes, ruling out IRE1-independent XBP1 splicing (FIG. 8E). Importantly, we ruled out the presence of a larger pool of IRE1 in S1R KO cells by treating cells with APY29, which forces IRE1-dependent XBP1 splicing (Korennykh et al., 2009). In this IRE1 stimulation paradigm, XBP1 slicing amounts were equal in both genotypes (FIG. 8F), indicating that S1R KO affects IRE1 activity, and not IRE1 protein abundance or substrate availability.

### Example 9

### S1R Critically Regulates Inflammatory Cytokine Production Via IRE1

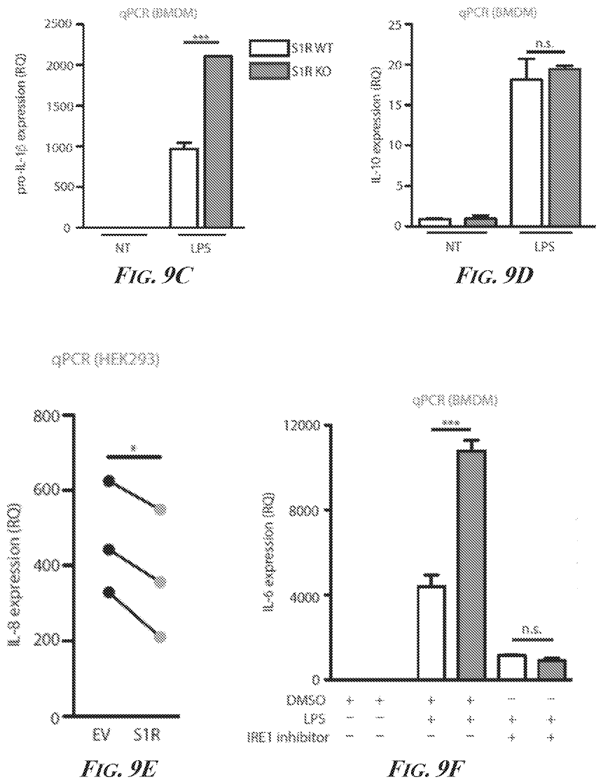

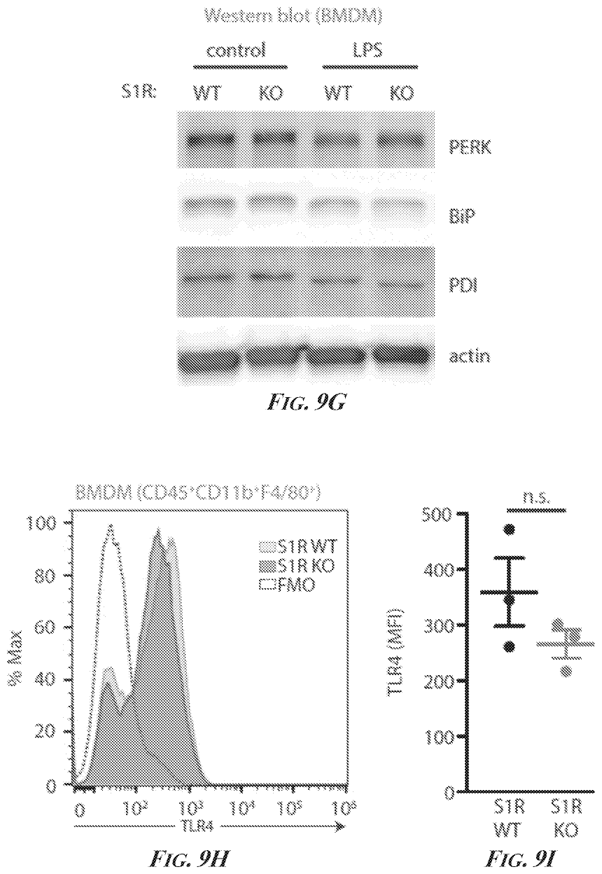

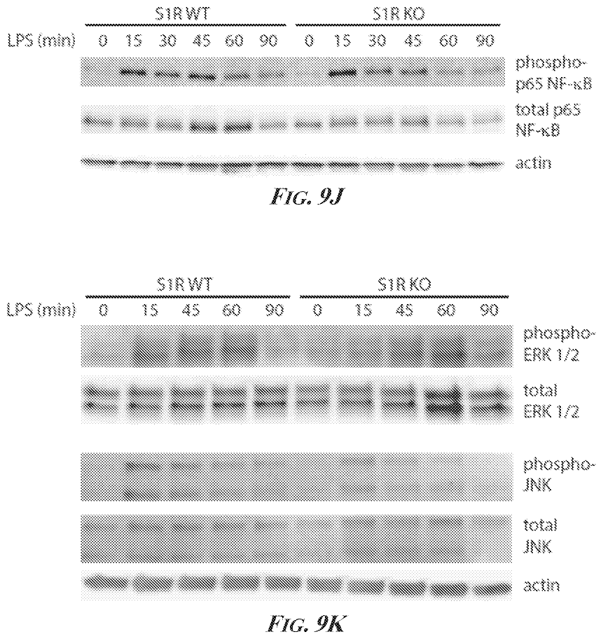

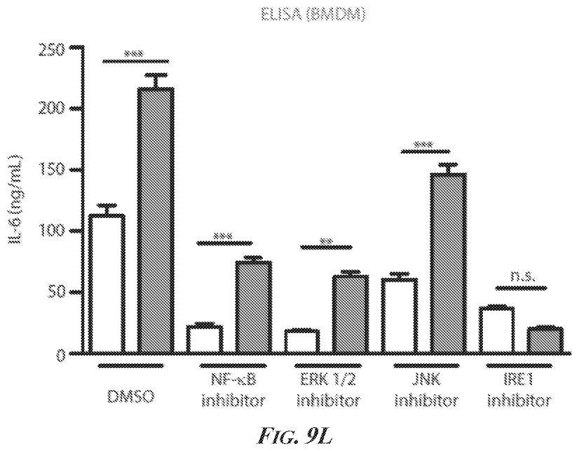

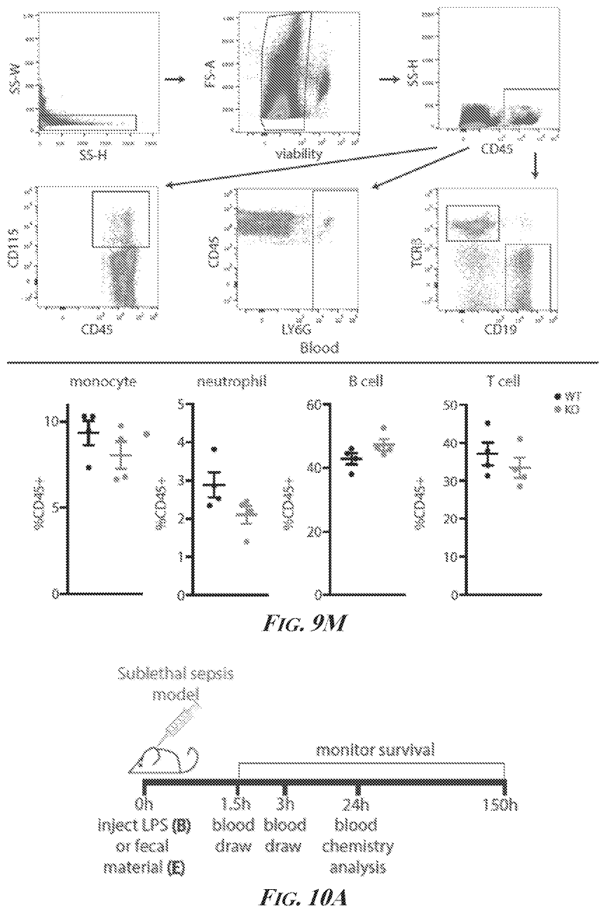

Because IRE1 activity is required for cytokine production (Urano et al., 2000; Martinon et al., 2010; Qiu et al., 2013), likely via XBP1 mediated transactivation of IL-6 and TNF-α, we next asked if S1R deficiency alters macrophage cytokine expression upon exposure to LPS. We found that S1R KO BMDM had elevated expression of IL-6 and pro-IL-1β transcripts and secreted higher amounts of IL-6 protein, when compared to wild type (WT) cells (FIGS. 9A-9C). However, S1R deficiency does not result in a global increase of cytokine production, as the anti-inflammatory cytokine IL-10 expression was unaffected in S1R KO BMDM (FIG. 9D). Having established that deletion of S1R leads to an increased inflammatory response, we examined whether overexpression of S1R could be anti-inflammatory. We overexpressed S1R in HEK293 that express mTLR4/MD2/CD14 and monitored expression of IL-8 following LPS treatment (Pozzobon et al., 2016). Relative to control transfected cells, overexpression of S1R resulted in a significant decrease in IL-8 production after LPS stimulation (p<0.05; FIG. 9E). These data collectively suggest that overexpression of S1R can dampen inflammation, whereas S1R deficiency contributes to the enhanced inflammatory response.

We next tested whether IRE1 endonuclease activity is responsible for the increase in proinflammatory cytokine expression in S1R KO cells. Pro-inflammatory cytokines, including IL-6, are rapidly induced by LPS in mice and humans, and correlate with poor prognosis in sepsis (Copeland et al., 2005b; Oda et al., 2005). We treated WT and S1R KO BMDM with LPS in the presence or absence of 4μ8C and analyzed IL-6 expression by qPCR. Inhibition of IRE1 endonuclease activity reversed IL-6 expression in KO cells to the amount observed in WT BMDM (FIG. 9F). Because S1R is an ER-resident protein, we wanted to rule out that deletion of S1R might result in global ER dysfunction (which could lead to the observed increase in IRE1 activation). To test this, we performed an immunoblot for ER-resident proteins that become upregulated during ER stress (Oslowski & Urano, 2011): Protein kinase R-like Endoplasmic Reticulum Kinase (PERK), binding immunoglobulin protein (BiP) and protein disulfide isomerase (PDI). We found comparable amounts of protein expression of all three proteins in S1R KO BMDM at baseline, with no change elicited by stimulation of BMDM with LPS (FIG. 9G). Therefore, we conclude that global ER stress does not drive IRE1 activity in S1R KO BMDM. Cell surface expression of TLR4 was unaffected by S1R deletion ruling out differential expression of the LPS receptor (FIGS. 9H and 9I). Activation of NF-κB (FIG. 9J), MAP kinase (ERK1/2) and the c-Jun N-terminal kinase (JNK; FIG. 9K) was identical between WT and S1R KO cells after LPS treatment. Finally, when we tested selective pharmacologic inhibitors of NF-κB, JNK, ERK1/2, and IRE1 for the ability to normalize LPS-induced IL-6 secretion in S1R KO BMDM, only the IRE1 inhibitor was effective at blunting the augmented inflammatory response of S1R KO cells (FIG. 9L). It is important to note that while NF-κB, JNK, and ERK1/2 inhibitors suppressed IL-6 production in both genotypes, S1R KO BMDM still produced elevated IL-6 relative to WT BMDM (FIG. 9L), indicating that these pathways are functional in S1R KO BMDM, but are not affected by deletion of S1R. We also cultured primary lung fibroblasts from the S1R KO and control mice and tested their response to LPS stimulation. Fibroblasts also presented with enhanced LPS-induced XBP1 splicing and inflammatory cytokine production that can be corrected by IRE1 inhibition (FIG. 9M). Taken together, these findings indicate that IRE1 signaling is selectively perturbed in S1R KO cells, and that the pro-inflammatory effects of S1R deletion likely depend on the endonuclease activity of IRE1.

### Example 10

### S1R Deficient Mice Display Increased Mortality in Sub-Lethal Models of Sepsis

To test the function of S1R in vivo, we subjected S1R KO mice to LPS injection, an animal model to study the inflammatory response to endotoxin in vivo (Lewis et al., 2016). S1R KO mice and WT littermate controls were injected with a sub-lethal dose of LPS (5 mg/kg) and survival was monitored for 6 days (FIG. 10A). WT animals experienced very low mortality (9%), whereas 62% of S1R KO mice succumbed to LPS-induced death (FIG. 10B), suggesting that S1R potently inhibits systemic inflammation. We next analyzed the concentration of pro-inflammatory cytokines TNF-α and IL-6 in serum at their reported peak expression, as these cytokines have been extensively shown to correlate with LPS-induced mortality (Remick et al., 2002; Copeland et al., 2005a). Peak serum TNF-α and IL-6 were significantly increased in LPS challenged S1R KO mice, when compared to controls (p<0.05; FIGS. 10C and 10D), whereas neither cytokine was detectable in the serum of unchallenged mice. To test if the increase in TNF-α and IL-6 in S1R KO mice was due to baseline differences in the composition of immune cells, we performed an immunophenotyping analysis of blood (FIGS. 10E and 10F), the peritoneal cavity (FIGS. 10D-10I) and immune organs (spleen and lymph nodes; FIGS. 10J and 10K). Our flow cytometry analyses revealed no significant differences in the innate and adaptive cell numbers and frequency, suggesting that S1R deficient mice do not have an apparent immune defect.

While LPS injection is a convenient model for study of endotoxin-mediated inflammation, the use of a single pathogen-associated molecular pattern (PAMP) does not fully recapitulate the biological complexity of sepsis. Therefore, we tested S1R KO mice in fecal-induced peritonitis (FIP), an alternate model of sepsis that involves injection of fecal material containing live bacteria (FIG. 10L; Shrum et al., 2014). Similar to our LPS challenge finding, WT mice receiving a sub-lethal dose of fecal slurry (1 g/kg of body weight) did not succumb to septic shock induced by FIP, whereas S1R KO mice experienced significant mortality (p<0.05; FIG. 10L). This increased mortality correlated with increased serum IL-6 (p<0.05; FIG. 10M) and significantly lowered core body temperature in S1R KO mice (p<0.01; FIG. 10N). S1R deficiency was also associated with elevated markers of organ failure, as revealed by serum chemistry analysis performed 24 hours after the initiation of septic shock. Indicators of impaired liver function alanine transferase (ALT, p<0.05; FIG. 10O) and aspartate aminotransferase (AST, p<0.001; FIG. 10P), indicator of kidney dysfunction, creatinine (p<0.001; FIG. 10Q), and indicator of heart dysfunction, creatine kinase (CK, p<0.01; FIG. 10R), were all significantly elevated in S1R deficient animals in both LPS and FIP models. Taken together, our data demonstrate increased susceptibility to models of sepsis and inflammation in S1R deficiency, characterized by elevated cytokines and multi-organ dysfunction.

### Example 11

### IRE1 Inhibition In Vivo is Protective in a Sepsis Model

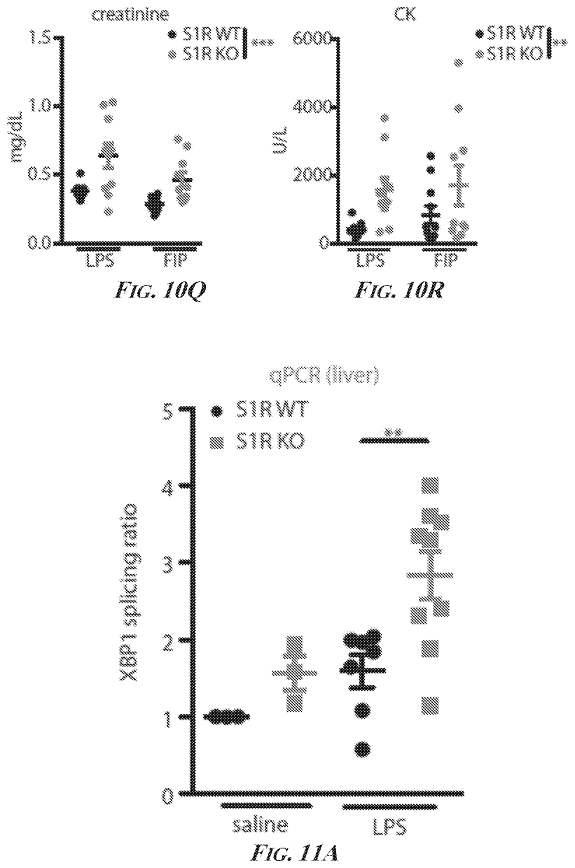

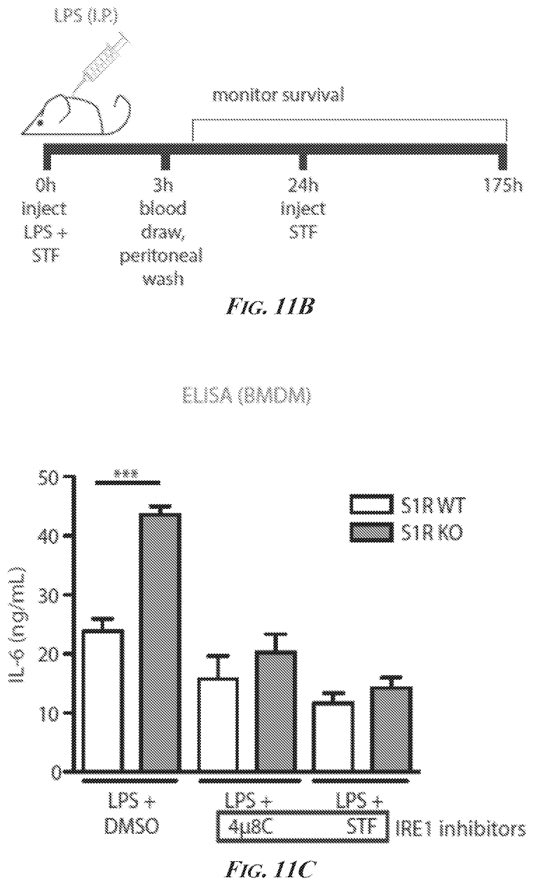

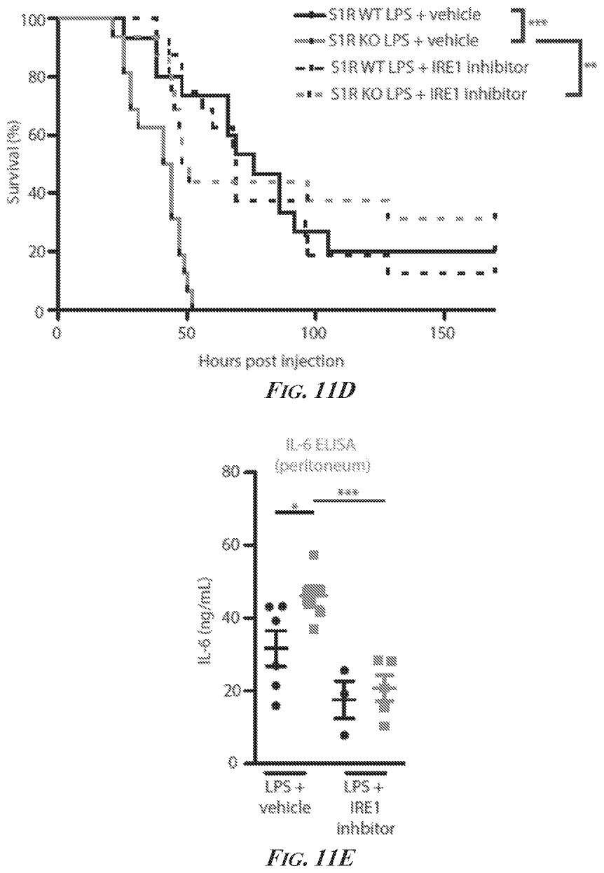

To test if LPS challenged S1R KO mice have increased IRE1 activity, we first examined XBP1 splicing in the liver, a key organ in the pathological progression of sepsis. LPS challenged S1R KO mice had increased hepatic XBP1 splicing when compared to WT mice (FIG. 11A). This finding suggests that similar IRE1-dependent inflammatory mechanisms we identified in cultured macrophages may be at work in vivo. If increased IRE1 activity is responsible for reduced survival of S1R KO mice during LPS challenge, then IRE1 inhibition should protect S1R KO mice subjected to LPS challenge (FIG. 11B). Due to the reported short half-life of the IRE1 inhibitor 4μ8C in vivo (Cross et al., 2012), we selected instead to use STF 083010 (herein referred to as STF), an effective IRE1 inhibitor (FIG. 11C) that has been used in in vivo studies (Papandreou et al., 2011; Cross et al., 2012). Again, LPS challenged S1R KO mice that received vehicle control experienced rapid mortality (FIG. 11D). Remarkably, STF administration (30 mg/kg at 0 hours and 24 hours) spared KO mice from LPS induced mortality (FIG. 11D), whereas it did not significantly affect the survival of WT mice. It is important to note that previous studies have indicated anti-inflammatory benefit of STF in S1R-expressing animals. Therefore, it is likely that, although we did not observe a statistically significant benefit in the paradigm used, dosing could likely be optimized to enhance the survival of WT mice as well.

The finding that an IRE1 inhibitor rescues S1R KO mice in a model of endotoxemia is in agreement with our hypothesis that cytokine production and LPS-induced mortality in S1R KO mice require excessive IRE1 endonuclease activity. Further supporting this hypothesis, we detected significantly higher IL-6 after 3 hours in the peritoneal exudate in LPS challenged vehicle-treated S1R KO mice compared to WT controls, which was corrected by STF treatment (p<0.05; FIG. 11E). A similar trend toward decreased IL-6 in serum by STF was also observed in the serum of S1R KO mice (FIG. 11F). In this treatment paradigm, we noted that vehicle (Kolliphor) treatment significantly increased LPS-induced IL-6 in the serum when compared to LPS alone (p<0.01; FIG. 11G). Because Kolliphor exacerbates LPS-induced inflammation, a lower dose of LPS was selected. Nevertheless, STF has a clear benefit in S1R KO survival. Importantly, injection of Kolliphor alone did not result in a detectable concentration of serum IL-6, nor did it cause any mortality in WT or KO mice, suggesting that vehicle treatment exacerbates LPS-induced inflammation, but is not inflammatory on its own (FIG. 11H). Collectively, these findings suggest that cytokine production and LPS-induced mortality in S1R KO mice require excessive IRE1 endonuclease activity.

### Example 12

### Therapeutic Administration of a S1R Ligand is Beneficial in a Preclinical Model of Sepsis and Human Cells

We next aimed to directly assess whether S1R function might be manipulated for benefit in an in vivo inflammatory context. We selected fluvoxamine (FLV), an antidepressant drug with low-nanomolar affinity for S1R, which has also been reported to have anti-inflammatory properties (Ghareghani et al., 2017). In order to elicit higher mortality in WT mice, we selected a higher dose of LPS (6 mg/kg) for this experiment, and first administered fluvoxamine (20 mg/kg) at the same time as LPS (FIG. 12A). Fluvoxamine treatment significantly protected WT mice from mortality and reduced serum IL-6 whereas, as expected, no significant effect was observed in S1R KO animals (p<0.05; FIGS. 12B and 12C). These results indicate that the anti-inflammatory effect of fluvoxamine is mediated by S1R.

We next tested whether fluvoxamine could be therapeutically administered to protect C57BL/6J from LPS administration or ongoing FIP sepsis model. Fluvoxamine was administered as indicated in FIG. 12D (90 min post LPS challenge) and FIG. 12E (30 min post FIP induction), after animals presented with a significant sickness behavior characterized by a decrease in body temperature (p<0.001; FIGS. 12F and 12G) and a clinical presentation of sepsis signs (p<0.01; FIGS. 12H and 12I). Therapeutic administration of fluvoxamine improved the clinical score (FIGS. 12H and 12I) and temperature (FIGS. 12J and 12K) of challenged animals. The treatment also significantly enhanced survival in both animal models (p<0.01; FIGS. 12L and 12M). Remarkably, fluvoxamine treatment was also beneficial in the FIP model when administered at an even later time point post FIP induction (90 min instead of 30 min; FIGS. 12N and 12O). To directly compare the effectiveness of fluvoxamine to the currently available therapeutics, we also administered Ceftriaxone, an antibiotic currently used as a standard of care for sepsis patients (Foster, Jr., 1991), 90 min post FIP induction. Fluvoxamine administration was as efficacious in enhancing survival as Ceftriaxone (CRO, 100 mg/kg; FIG. 12O), and the combination of fluvoxamine and ceftriaxone further improved survival in the FIP model of sepsis, without reaching significance when compared to single treatment (FIG. 12O). These data suggest the potential for therapeutic targeting of the S1R as an alternate or additive avenue in sepsis treatment.

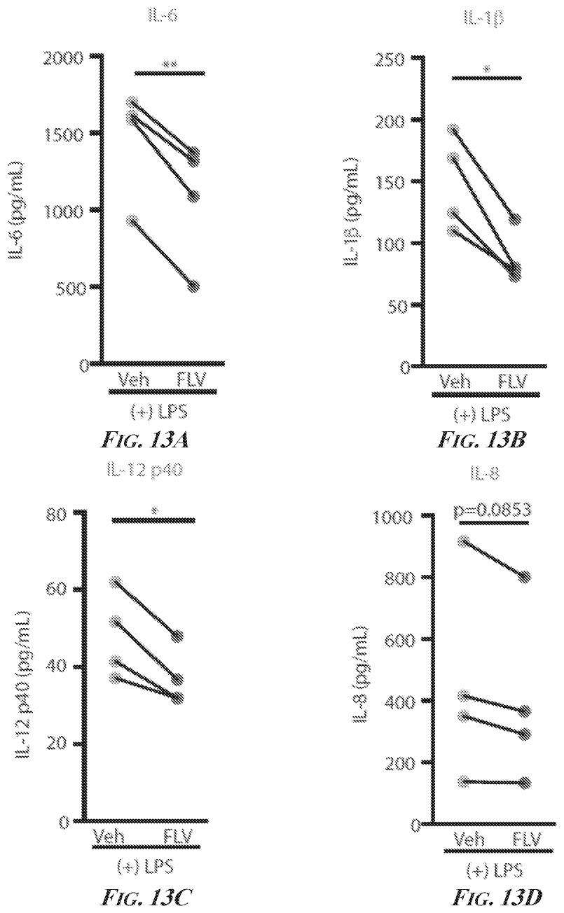

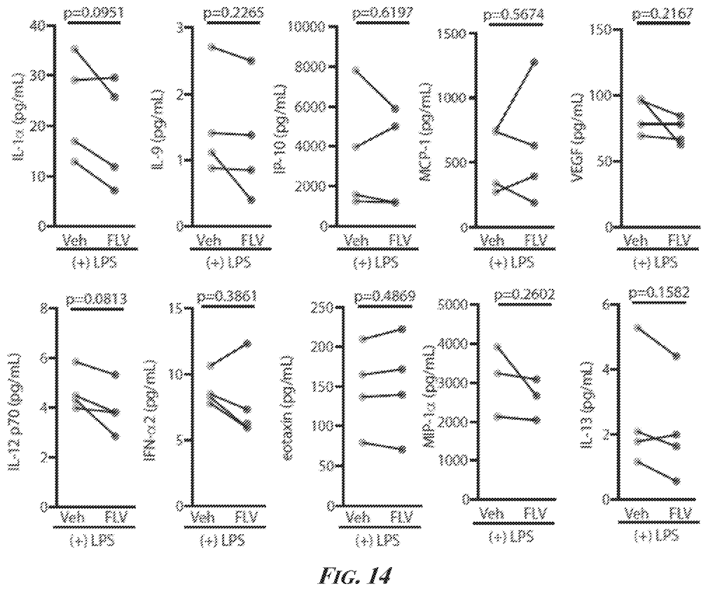

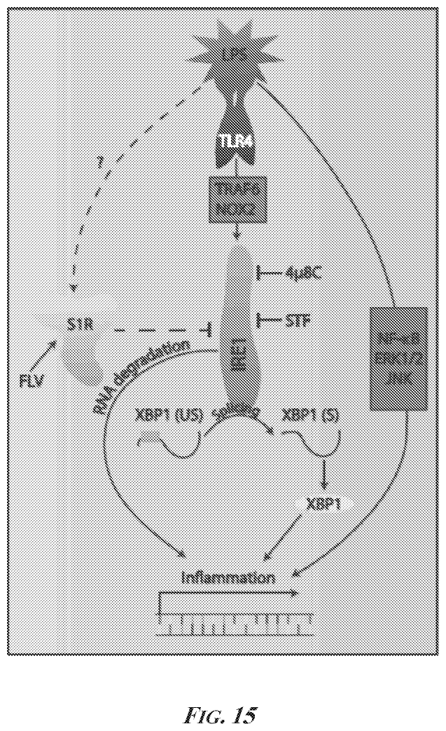

To assess whether targeting S1R can dampen inflammation in human cells, heparinized peripheral blood from healthy donors was stimulated ex vivo with LPS (10 ng/ml) in the presence or absence of fluvoxamine (20 μM) and the production of inflammatory mediators was measured by multiplex analysis. Fluvoxamine significantly reduced LPS-induced IL-6 (p<0.01; FIG. 13A), IL-1β (p<0.05; FIG. 13B), and IL-12 p40 (p<0.01; FIG. 13C), and decreased IL-8 (FIG. 13D) production in cells from all donors analyzed. These data indicate that the anti-inflammatory action of this S1R ligand is likely conserved across species. Importantly, modulation of S1R during LPS treatment was limited to a subset of inflammatory mediators (FIG. 14) and was not the result of global cytokine suppression. Taken together, our data show that fluvoxamine can influence the inflammatory response in murine and human cells in a S1R dependent manner and suggest that therapeutic exploitation of S1R targeting might hold promise for the control of inflammatory insults.

### Discussion of Examples 8-12

The ER stress sensing protein IRE1α (as well as the closely related protein IRE1β) is able to powerfully affect the inflammatory behavior of both immune and non-immune cells in numerous contexts (Cho et al., 2013; Janssens et al., 2014). However, little is yet known about the factors that modulate the extent of IRE1 signaling during inflammation. Here we identify S1R as a regulator of IRE1 endonuclease function during LPS-induced inflammation (FIG. 15). S1R and IRE1 may associate both basally and after LPS stimulation, suggesting that S1R is uniquely poised to sensitively control IRE1 activity. This finding is particularly exciting, since the physiological and pathophysiological relevance of IRE1 is well established (Janssens et al., 2014). However, direct therapeutic targeting of IRE1 has been met with significant challenges (Lipson et al., 2006; Fu et al., 2012), and alternative routes toward IRE1 signaling modulation are sought after. We propose that S1R, which can be targeted by several drugs that are already in clinical use, might represent one such option.

One important caveat of our study is that, while preclinical models of septic shock are convenient for the discovery of new therapeutic treatments for sepsis, they incompletely replicate the diversity of human sepsis and translational efficacy of preclinical findings in human patients is difficult to predict. There are likely many factors contributing to this challenge, including diversity of predisposition and underlying physiological features, heterogeneous pathophysiology, and variability of causative infectious agents (Levy et al., 2003). Clinically defining such a broad array of processes as one syndrome, sepsis, complicates the application of novel therapeutic approaches. Work to more effectively design clinical sepsis studies to properly apply preclinical findings is an active area of the sepsis field (Davenport et al., 2016). At the same time, novel preclinical sepsis models and methods are arising that may allow for experimentation in a wider range of conditions associated with sepsis (Hu et al., 2012). Despite these challenges, preclinical sepsis models have shown significant promise, and more precise classification of sepsis conditions may lead to efficacious application of interventions that have been identified in animal models.

Here we show that S1R deficiency appears to selectively enhance activity of IRE1 and does not influence other inflammatory pathways, including NF-κB, JNK and ERK. The ability of the S1R-IRE1 interaction to influence immune and non-immune cell activity may prove to be of importance in inflammatory and degenerative diseases in which S1R and IRE1 dysfunction have been implicated, including Alzheimer's disease (Marrazzo et al., 2005; Salminen et al., 2009) and amyotrophic lateral sclerosis (Kikuchi et al., 2006; Mavlyutov et al., 2015). Additionally, although our study focused on XBP1 splicing as an indicator of IRE1 endonuclease activity, IRE1 can also cleave other RNA species (in a process called regulated IRE1-dependent decay, or RIDD), which may drive inflammation as well (Cho et al., 2013). Some of our observed findings may be a result of RIDD and may not depend on XBP1. Another potential mechanism is that S1R may be altering calcium signaling, as S1R has been shown to modulate the conductivity of inositol triphosphate receptor (IP3R; Hayashi & Su, 200738). However, several lines of evidence suggest that changes in calcium signaling do not produce the observed inflammatory effects. First, we show that the cytosolic inflammatory signaling proteins NF-κB, JNK, and ERK1/2, all of which are sensitive to changes in calcium, are not affected by S1R deletion. Additionally, calcium dysregulation might inhibit protein folding, but we do not observe changes in ER chaperone abundance basally or after LPS stimulation of S1R KO BMDM, again suggesting that S1R does not strongly perturb calcium homeostasis in our cells. Further studies examining calcium flux in S1R KO BMDM might reveal additional important features of S1R function, such as during prolonged ER stress, but calcium flux does not appear to be central to the pathway described in this study.

One limitation of our study is that we have not fully elucidated the mechanism by which S1R controls IRE1. While we have demonstrated with our proximity ligation approach that S1R and IRE1 were in close proximity and that interaction was further promoted upon LPS stimulation, our study did not address if intermediates were necessary for the formation of the putative S1R-IRE1 complex. Our attempts at coimmunoprecipitation did not convincingly demonstrate a strong association between endogenous S1R and IRE1, unlike what has been described with over-expressed proteins (Mori et al., 2013). This suggests that the interaction might be transient and/or require intermediates. Furthermore, our study did not explore what are the signaling events that control S1R activity in the context of inflammation. To date, only one endogenous S1R ligand has been identified, N,N-Dimethyl tryptamine (DMT), a tryptophan metabolite, known for its psychedelic activity (Fontanilla et al., 2009). Limited evidence suggests that DMT-S1R interactions can influence the activation of immune cells (Szabo et al., 2014). Perhaps, understanding the interaction of S1R and its endogenous ligands that are regulated by inflammation could help answer this open question. Although we found that the anti-inflammatory action of fluvoxamine depends on S1R in our animal models of sepsis and inflammation, there is still much to be learned about fluvoxamine and other Selective Serotonin Reuptake Inhibitors (SSRIs) in inflammation. Some SSRIs that do not have affinity for S1R also have reported anti-inflammatory properties, linking serotonin signaling in immune cells to inflammation (Shajib & Khan, 2014). Furthermore, it remains unknown whether the requirement for S1R in fluvoxamine efficacy is due to direct binding of fluvoxamine to S1R. Nonetheless, it will be important to consider S1R when studying SSRIs in inflammation.

Summarily, the endoplasmic reticulum (ER) is classically defined as the site of secreted protein synthesis and trafficking. Recently, the ER functions have been extended to a plethora of new biological roles, including inflammation. Activation of the ER stress sensor IRE1 is essential for the normal inflammatory response to stimuli such as LPS. However, the mechanisms by which IRE1 regulates inflammation remain unclear. Here, we identify a novel role of the ER protein Sigma-1 receptor (S1R) as a critical inhibitor of LPS-induced cytokine production. Mice lacking S1R succumb quickly to hypercytokinemia after administration of a sub-lethal dose of LPS. Mechanistically, S1R controls IRE1 endonuclease activity required for cytokine expression and regulates the biosynthesis of IRE1, without an impact on cytosolic inflammatory signaling pathways. Our data reveal the contribution of S1R to the restraint of the inflammatory response, and place S1R as a promising therapeutic target to treat inflammatory disorders.

### REFERENCES

All references listed below, as well as all references cited in the instant disclosure, including but not limited to all patents, patent applications and publications thereof, scientific journal articles, and database entries (e.g., GENBANK® and UniProt biosequence database entries and all annotations available therein) are incorporated herein by reference in their entireties to the extent that they supplement, explain, provide a background for, or teach methodology, techniques, and/or compositions employed herein.
Abuaita et al. (2015) The Endoplasmic Reticulum Stress Sensor Inositol-Requiring Enzyme 1 alpha Augments Bacterial Killing through Sustained Oxidant Production. MBio 6:e00705.
Altschul et al. (1990a) Basic local alignment search tool. J Mol Biol 215:403-410.
Altschul et al. (1990b) Protein database searches for multiple alignments. Proc Natl Acad Sci USA 87:14:5509-5513.
Altschul et al. (1997) Gapped BLAST and PSI-BLAST: a new generation of protein database search programs. Nucleic Acids Res 25:3389-3402.
Ausubel et al. (1995) Current Protocols in Molecular Biology, Greene Publishing.
Blok et al. (1981) The effect of chloroquine on lysosomal function and cell-coat glycoprotein transport in the absorptive cells of cultured human small-intestinal tissue. Cell Tissue Res 218:227-251.
Chen & Brandizzi (2013) IRE1: ER stress sensor and cell fate executor. Trends Cell Biol 23:547-555.
Cho et al. (2013) The unfolded protein response element IRE1alpha senses bacterial proteins invading the ER to activate RIG-I and innate immune signaling. Cell Host Microbe 13:558-569.
Chou & Fasman (1974) Prediction of protein conformation. Biochemistry 13:222-245.
Chou & Fasman (1978) Empirical predictions of protein conformation. Ann Rev Biochem 47:251-276.
Chou & Fasman (1979) Prediction of beta-turns. Biophys J 26:367-384.
Cobos et al. (2008) Pharmacology and therapeutic potential of sigma(1) receptor ligands. Current Neuropharmacology 6:344-366.
Copeland et al. (2005a) Acute inflammatory response to endotoxin in mice and humans. Clin Diagn Lab Immunol 12:60-67.
Copeland et al. (2005b) Inflammation, I. the Host Response to Injury, Acute inflammatory response to endotoxin in mice and humans. Clinical and Diagnostic Laboratory Immunology 12:60-67.
Cross et al. (2012) The molecular basis for selective inhibition of unconventional mRNA splicing by an IRE1-binding small molecule. Proc Natl Acad Sci USA 109:E869-878.
Cubillos-Ruiz et al. (2015) ER Stress Sensor XBP1 Controls Anti-tumor Immunity by Disrupting Dendritic Cell Homeostasis. Cell 161:1527-1538.
Dalton & Barton (2014) Over-expression of secreted proteins from mammalian cell lines. Protein Sci 23:517-525.
Davenport et al. (2016) Genomic landscape of the individual host response and outcomes in sepsis: a prospective cohort study. Lancet Respir Med 4:259-271.
Fenteany & Schreiber (1998) Lactacystin, proteasome function, and cell fate. J Biol Chem 273:8545-8548.
Fontanilla et al. (2009) The hallucinogen N,N-dimethyltryptamine (DMT) is an endogenous sigma-1 receptor regulator. Science 323:934-937.
Foster, Jr. (1991) Ceftriaxone in treatment of serious infections. Septicemia. Hosp Pract (Off Ed) 26 Suppl 5:43-47; discussion 62-43.
Fu et al. (2012) The role of endoplasmic reticulum in hepatic lipid homeostasis and stress signaling. Cell Metab 15:623-634.
Gait (1984) Oligonucleotide Synthesis: A Practical Approach, IRL Press, Oxford, England.
Gao et al. (2008) Synoviolin promotes IRE1 ubiquitination and degradation in synovial fibroblasts from mice with collagen-induced arthritis. EMBO Rep 9:480-485.
Gaultier et al. (2008) Regulation of tumor necrosis factor receptor-1 and the IKK-NF-kappaB pathway by LDL receptor-related protein explains the antiinflammatory activity of this receptor. Blood 111:5316-5325.
Gaultier et al. (2009) Low-density lipoprotein receptor-related protein 1 is an essential receptor for myelin phagocytosis. J Cell Sci 122:1155-1162.
Gaultier et al. (2010) LDL receptor-related protein 1 regulates the abundance of diverse cell-signaling proteins in the plasma membrane proteome. J Proteome Res 9:6689-6695.
Gennaro (1990) Remington's Pharmaceutical Sciences, 18th ed., Mack Pub. Co., Easton, Pa., United States of America.
Gennaro (ed.) (2003) Remington: The Science and Practice of Pharmacy, 20th edition Lippincott, Williams & Wilkins, Philadelphia, Pa., United States of America.
Ghareghani et al. (2017) Fluvoxamine stimulates oligodendrogenesis of cultured neural stem cells and attenuates inflammation and demyelination in an animal model of multiple sclerosis. Sci Rep 7:4923.
Girard et al. (2009) Expression of an activating mutation in the gene encoding the KATP channel subunit Kir6.2 in mouse pancreatic beta cells recapitulates neonatal diabetes. J Clin Invest 119:80-90.
Glauser et al. (1991) Septic shock: pathogenesis. Lancet 338:732-736.
Glover (1985) DNA Cloning: a Practical Approach. Oxford Press, Oxford.
Green et al., eds. (2014) Molecular Cloning: A Laboratory Manual, Cold Spring Harbor Laboratory Press, Cold Spring Harbor, N.Y., United States of America.
Griffiths-Jones (2004) The microRNA Registry. Nucleic Acids Res 32:D109-D111.
Griffiths-Jones et al. (2006) miRBase: microRNA sequences, targets and gene nomenclature. Nucleic Acids Res 34:D140-D144.
Griffiths-Jones et al. (2008) miRBase: tools for microRNA genomics. Nucleic Acids Res 36:D154-D158.
Grimm et al., (1999) Titration of AAV-2 particles via a novel capsid ELISA: packaging of genomes can limit production of recombinant AAV-2. Gene Ther 6:1322-1330.
Gross & Mienhofer (eds.) (1981) The Peptides, Vol. 3. Academic Press, New York, N.Y., United States of America, pp. 3-88.
Ha et al. (2011) Late-onset inner retinal dysfunction in mice lacking sigma receptor 1 (sigmaR1). Invest Ophthalmol Vis Sci 52:7749-7760.
Ha et al. (2014) Sigma receptor 1 modulates ER stress and Bcl2 in murine retina. Cell Tissue Res 356:15-27.
Harlow & Lane (1988) Antibodies, a Laboratory Manual, Cold Spring Harbor Laboratory Publications, Cold Spring Harbor, N.Y., United States of America.
Hashimoto (2015) Activation of sigma-1 receptor chaperone in the treatment of neuropsychiatric diseases and its clinical implication. J Pharmacol Sci 127:6-9.
Hayashi & Su (2007) Sigma-1 receptor chaperones at the ER-mitochondrion interface regulate Ca(2+) signaling and cell survival. Cell 131:596-610.
Hetz (2012) The unfolded protein response: controlling cell fate decisions under ER stress and beyond. Nat Rev Mol Cell Biol 13:89-102.
Hetz et al. (2006) Proapoptotic BAX and BAK modulate the unfolded protein response by a direct interaction with IRE1alpha. Science 312:572-576.
Hsieh et al. (2003) Mesd encodes an LRP5/6 chaperone essential for specification of mouse embryonic polarity. Cell 112:355-367.
Hu et al. (2012) When host defense goes awry: Modeling sepsis-induced immunosuppression. Drug Discov Today Dis Models 9:e33-e38.
Janssens et al. (2014) Emerging functions of the unfolded protein response in immunity. Nat Immunol 15:910-919.
Jiang et al. (2016) Acridine derivatives as inhibitors of the IRE1alpha-XBP1 pathway are cytotoxic to human multiple myeloma, Mol. Cancer Ther. 15 (9):2055-2065.
Karlin & Altschul (1990) Methods for assessing the statistical significance of molecular sequence features by using general scoring schemes. Proc Natl Acad Sci USA 87:2264-2268.
Karlin & Altschul (1993) Applications and statistics for multiple high-scoring segments in molecular sequences. Proc Natl Acad Sci USA 90:5873-5877.
Kikuchi et al. (2006) Spinal cord endoplasmic reticulum stress associated with a microsomal accumulation of mutant superoxide dismutase-1 in an ALS model. Proc Natl Acad Sci USA 103:6025-6030.
Kohlbrenner et al. (2012) Quantification of AAV particle titers by infrared fluorescence scanning of coomassie-stained sodium dodecyl sulfate-polyacrylamide gels. Hum Gene Ther Meth 23:198-203.
Korennykh et al. (2009) The unfolded protein response signals through high-order assembly of Irel. Nature 457:687-693.
Kourrich et al. (2012) The sigma-1 receptor: roles in neuronal plasticity and disease. Trends Neurosci 35:762-771.
Kozomara et al. (2011) miRBase: integrating microRNA annotation and deep-sequencing data. Nucleic Acids Res 39:D152-D157
Kozomara et al. (2014) miRBase: annotating high confidence microRNAs using deep sequencing data. Nucleic Acids Res 42:D68-D73.
Kozomara et al. (2019) miRBase: from microRNA sequences to function. Nucleic Acids Res 47:D155-D162.
Kumar & Valdivia (2008) Actin and intermediate filaments stabilize the Chlamydia trachomatis vacuole by forming dynamic structural scaffolds. Cell Host Microbe 4:159-169.
Lawrence (2009) The nuclear factor NF-kappaB pathway in inflammation. Cold Spring Harb Perspect Biol 1:a001651.
Levy et al. (2003) Sccm/Esicm/Accp/Ats/Sis, 2001 SCCM/ESICM/ACCP/ATS/SIS International Sepsis Definitions Conference. Crit Care Med 31:1250-1256.
Lewis et al. (2016) Current Murine Models of Sepsis. Surg Infect (Larchmt) 17:385-393.
Li et al. (2011) Unfolded protein response in cancer: the physician's perspective, J. Hematol. Oncol. 4:8.
Lipson et al. (2006) Regulation of insulin biosynthesis in pancreatic beta cells by an endoplasmic reticulum-resident protein kinase IRE1. Cell Metab 4:245-254.
Lipson et al. (2008) The role of IRE1alpha in the degradation of insulin mRNA in pancreatic beta-cells. PLoS One 3:e1648.
Lu et al. (2008) LPS/TLR4 signal transduction pathway. Cytokine 42:145-151.
Marrazzo et al. (2005) Neuroprotective effects of sigma-1 receptor agonists against beta-amyloid-induced toxicity. Neuroreport 16:1223-1226.
Martinon et al. (2010) TLR activation of the transcription factor XBP1 regulates innate immune responses in macrophages. Nat Immunol 11:411-418.
Maurice & Goguadze (2017) Sigma-1 (cri) Receptor in Memory and Neurodegenerative Diseases. Handb Exp Pharmacol, Colume 244, Springer, Cham.
Mavlyutov et al. (2015) Role of the Sigma-1 receptor in Amyotrophic Lateral Sclerosis (ALS). J Pharmacol Sci 127:10-16.
Medzhitov (2008) Origin and physiological roles of inflammation. Nature 454:428-435.
Meunier & Hayashi (2010) Sigma-1 receptors regulate Bcl-2 expression by reactive oxygen species-dependent transcriptional regulation of nuclear factor kappaB. J Pharmacol Exp Ther 332:388-397.
Mimura et al. (2012) Blockade of XBP1 splicing by inhibition of IRE1alpha is a promising therapeutic option in multiple myeloma, Blood 119 (24):5772-5781.
Mori et al. (2013) Sigma-1 receptor chaperone at the ER-mitochondrion interface mediates the mitochondrion-ER-nucleus signaling for cellular survival. PLoS One 8:e76941.
Morito & Nagata (2012) ER Stress Proteins in Autoimmune and Inflammatory Diseases. Front Immunol 3:48.
Moritz et al. (2015) Live imaging reveals a new role for the sigma-1 (sigma1) receptor in allowing microglia to leave brain injuries. Neurosci Lett 591:13-18.
Nguyen et al. (2015) Role of sigma-1 receptors in neurodegenerative diseases. J Pharmacol Sci 127:17-29.
Oda et al. (2005) Sequential measurement of IL-6 blood levels in patients with systemic inflammatory response syndrome (SIRS)/sepsis. Cytokine 29:169-175.
Ojha & Amaravadi (2017), Pharmacological Research 120:258-266.
Oslowski & Urano (2011) Measuring ER stress and the unfolded protein response using mammalian tissue culture system. Methods Enzymol 490:71-92.
Papandreou et al. (2011) Identification of an Ire1alpha endonuclease specific inhibitor with cytotoxic activity against human multiple myeloma. Blood 117:1311-1314.
Pozzobon et al. (2016) Treponema pallidum (syphilis) antigen TpF1 induces angiogenesis through the activation of the IL-8 pathway. Scientific Reports 6:18785.
Qiu et al. (2013) Toll-like receptor-mediated IRE1 alpha activation as a therapeutic target for inflammatory arthritis. EMBO J 32:2477-2490.
Remick et al. (2002) Six at six: interleukin-6 measured 6 h after the initiation of sepsis predicts mortality over 3 days. Shock 17:463-467.
Ri et al. (2012) Identification of Toyocamycin, an agentcytotoxic for multiple myeloma cells, as a potent inhibitor of ERstress-induced XBP1 mRNA splicing, Blood Cancer J. 2 (7):e79.
Roe et al. (1996) DNA Isolation and Sequencing: Essential Techniques, John Wiley, New York, N.Y., United States of America.
Roux et al. (2012) A promiscuous biotin ligase fusion protein identifies proximal and interacting proteins in mammalian cells. J Cell Biol 196:801-810.
Ruland (2011) Return to homeostasis: downregulation of NF-kappaB responses. Nat Immunol 12:709-714.
Sabino et al. (2009) Sigma-1 receptor knockout mice display a depressive-like phenotype. Behav Brain Res 198:472-476.
Salminen et al. (2009) ER stress in Alzheimer's disease: a novel neuronal trigger for inflammation and Alzheimer's pathology. J Neuroinflammation 6:41.
Sambrook et al. (1989) Molecular Cloning: A Laboratory Manual, Cold Spring Harbor Laboratory Publications, Cold Spring Harbor, N.Y., United States of America.
Sanches et al. (2014) Structure and mechanism of action of the hydroxy-aryl-aldehyde class of IRE1 endoribonucleaseinhibitors, Nat. Commun. 5:4202.
Seki et al. (2017) Lineage-Specific Metabolic Properties and Vulnerabilities of T Cells in the Demyelinating Central Nervous System. J Immunol 198:4607-4617.
Seluanov et al. (2010) Establishing primary adult fibroblast cultures from rodents. J Vis Exp 44:e2033.
Shajib & Khan (2014) The role of serotonin and its receptors in activation of immune responses and inflammation. Acta Physiol (Oxf) 213:561-574.
Shin et al. (2004) Inhibitory action of novel aromatic diamine compound on lipopolysaccharide-induced nuclear translocation of NF-kappaB without affecting IkappaB degradation. FEBS Lett 571:50-54.
Shrum et al. (2014) A robust scoring system to evaluate sepsis severity in an animal model. BMC Research Notes 7:233.
Szabo et al. (2014) Psychedelic N,N-dimethyltryptamine and 5-methoxy-N,N-dimethyltryptamine modulate innate and adaptive inflammatory responses through the sigma-1 receptor of human monocyte-derived dendritic cells. PLoS One 9:e106533.
Tang et al. (2014) Inhibition of ER stress-associated IRE-1/XBP-1 pathway reduces leukemic cell survival, J. Clin. Invest. 124 (6):2585-2598.
Tchedre & Yorio (2008) sigma-1 receptors protect RGC-5 cells from apoptosis by regulating intracellular calcium, Bax levels, and caspase-3 activation. Invest Ophthalmol Vis Sci 49:2577-2588.
Thurm & Halsey (2005) Measurement of cytokine production using whole blood. Curr Protoc Immunol Chapter 7, Unit 7 18B.
Tirasophon et al. (2000) The endoribonuclease activity of mammalian IRE1 autoregulates its mRNA and is required for the unfolded protein response. Genes Dev 14:2725-2736.
U.S. Patent Application Publication Nos. 2014/0296211; 2015/0073046; 2016/0009674; 2017/0360798; 2019/0000991; 2019/0008909.
U.S. Pat. No. 9,750,746.
Urano et al. (2000) Coupling of stress in the ER to activation of JNK protein kinases by transmembrane protein kinase IRE1. Science 287:664-666.
Vallabhapurapu & Karin (2009) Regulation and function of NF-kappaB transcription factors in the immune system. Annu Rev Immunol 27:693-733.
Vandesompele et al. (2002) Accurate normalization of real-time quantitative RT-PCR data by geometric averaging of multiple internal control genes. Genome Biol 3:research0034.1.
Volkmann et al. (2011) Potent and selective inhibitors of the inositol-requiring enzyme 1 endoribonuclease, J. Biol. Chem. 286 (14):12743-12755.
Yao et al. (2010) Molecular mechanisms involving sigma receptor-mediated induction of MCP-1: implication for increased monocyte transmigration. Blood 115:4951-4962.
Yoshida (2007) ER stress and diseases. FEBS J 274:630-658.
Zhang et al. (2008) The isolation and characterization of murine macrophages. Curr Protoc Immunol Chapter 14:Unit 14 11.
Zhang et al. (2017) Sigma-1 receptor deficiency reduces GABAergic inhibition in the basolateral amygdala leading to LTD impairment and depressive-like behaviors. Neuropharmacology 116:387-398.

While the presently disclosed subject matter has been disclosed with reference to specific embodiments, it is apparent that other embodiments and variations of the presently disclosed subject matter may be devised by others skilled in the art without departing from the true spirit and scope of the presently disclosed subject matter.

1. A method for treating inflammation in a subject in need thereof, the method comprising administering to the subject an effective amount of a Sigma-1 receptor (S1R) activity modulator to thereby treat inflammation in the subject.

2. The method of claim 1, wherein modulation of S1R activity treats conditions where an inflammatory response in a subject is detrimental or impaired.

3. The method of claim 1, wherein the inflammation is associated with septic shock.

4. The method of claim 1, wherein the S1R activity modulator is a S1R agonist.

5. The method of claim 4, wherein the S1R agonist is selected from the group consisting of wherein is a S1R agonist is selected from the group consisting of PRS-013, SA-4503, siramesine, (+)-pentazocine, (+)-SKF10,047, PRE084 (2-morpholin-4-ylethyl 1-phenylcyclohexane-1-carboxylate), SA4503 (1-4-β-phenylpropyl)piperazine), (±)-PPCC oxalate, PRE-084 hydrochloride, SA 4503 dihydrochloride, (+)-SK&F 10047 hydrochloride, and a compound commercially available under the following trade names ANAVEX® 2-73 (1-(2,2-diphenyltetrahydro-3-furanyl)-N,N-dimethylmethanamine), ANAVEX® 3-71 (1-(2,8-dimethyl-1-thia-3,8-diazaspiro(4.5)dec-3-yl)-3-(1H-indol-3-yl)propan-1-one), ANAVEX® 1-51, ANAVEX® 1079, ANAVEX® 1067, ANAVEX® 1037, ANAVEX® 1519, and ANAVEX® 1066.

6. The method of claim 1, wherein the S1R activity modulator is a composition that increases a level of S1R in the subject.

7. The method of claim 6, wherein the composition that increases a level of S1R in the subject comprises an expression vector that expresses S1R.

8. The method of claim 1, wherein the S1R activity modulator comprises an oligonucleotide, optionally an miRNA.

9. The method of claim 1, wherein two or more S1R activity modulators are administered in combination.

10. The method of claim 1, further comprising administering an additional therapeutic agent.

11. The method of claim 10, wherein the additional therapeutic agent is an IRE1 specific endonuclease inhibitor.

12. The method of claim 11, the IRE1 specific endonuclease inhibitor is selected from the group consisting of 4μ8C (7-Hydroxy-4-methyl-2-oxo-2H-1-benzopyran-8-carboxaldehyde), STF 083010 (N-2-thiophenesulfonamide), MKC8866 (CAS #1338934-59-0), Kira 6 (CAS #1589527-65-0), Kira 8 (CAS #1630086-20-2), MKC3946 (CAS #1093119-54-0), GSK2850163 (CAS #2121989-91-9), 6-Bromo-2-hydroxy-3-methoxybenzaldehyde (CAS #20035-41-0), 3-methoxy-6-bromosalicylaldehyde salicylaldimines, toyocamycin, N⁹-β-(dimethylamino) propyl)-N³,N³,N⁶,N⁶-tetramethylacridine-3,6,9-triamine (3,6-DMAD), Hydroxy-aryl-aldehydes (HAA), and irestatin.

13. (canceled)

14. (canceled)

15. A pharmaceutical composition comprising, consisting essentially of, or consisting of an effective amount of a Sigma-1 receptor (S1R) activity modulator to treat inflammation in a subject in need thereof.

16. The composition of claim 13, wherein modulation of S1R activity treats conditions where an inflammatory response in a subject is detrimental or impaired.

17. The composition of claim 13, wherein the inflammation is associated with septic shock.

18. The composition of claim 13, wherein the S1R activity modulator is a S1R agonist.

19. The composition of claim 18, wherein the S1R agonist is selected from the group consisting of PRS-013, SA-4503, siramesine, (+)-pentazocine, (+)-SKF10,047, PRE084 (2-morpholin-4-ylethyl 1-phenylcyclohexane-1-carboxylate), SA4503 (1-4-(3-phenylpropyl)piperazine), (±)-PPCC oxalate, PRE-084 hydrochloride, SA 4503 dihydrochloride, (+)-SK&F 10047 hydrochloride, and a compound commercially available under the following trade names ANAVEX® 2-73, ANAVEX® 3-71, ANAVEX® 1-51, ANAVEX® 1079, ANAVEX® 1067, ANAVEX® 1037, ANAVEX® 1519, and ANAVEX® 1066.

20. The composition of claim 13, wherein the S1R activity modulator is a composition that increases a level of S1R in the subject.

21. The composition of claim 20, wherein the composition that increases a level of S1R in the subject comprises an expression vector that expresses S1R.

22. The composition of claim 13, wherein the S1R activity modulator comprises an oligonucleotide.

23. The composition of claim 13, wherein two or more S1R activity modulators are provided in combination.

24. The composition of claim 13, further comprising providing an additional therapeutic agent.

25. The composition of claim 24, wherein the additional therapeutic agent is an IRE1 specific endonuclease inhibitor.

26. The composition of claim 25, wherein the IRE1 specific endonuclease inhibitor is selected from the group consisting of 4μ8C (7-Hydroxy-4-methyl-2-oxo-2H-1-benzopyran-8-carboxaldehyde), STF 083010 (N-2-thiophenesulfonamide), MKC8866 (CAS #1338934-59-0), Kira 6 (CAS #1589527-654)), Kira 8 (CAS #1630086-20-2), MKC3946 (CAS #1093119-54-0), GSK2850163 (CAS #2121989-91-9), 6-Bromo-2-hydroxy-3-methoxybenzaldehyde (CAS #20035-41-0), 3-methoxy-6-bromosalicylaldehyde salicylaldimines, toyocamycin, N⁹-β-(dimethylamino) propyl)-N³,N³,N⁶,N⁶-tetramethylacridine-3,6,9-triamine (3,6-DMAD), Hydroxy-aryl-aldehydes (HAA), and irestatin.


SEQUENCE LISTING (11 sequences)

SEQ ID NO: 1 - DNA, 1672 bp, Homo sapiens
  CDS (91)..(762)
gtgggcagccggcgggctccgaggccgtgagcgcaaagcctcaggccccggctccctcctgagctgcgccgtgccaggccgcccgccgggatgcagtgggccgtgggccggcggtgggcgtgggccgcgctgctcctggctgtcgcagcggtgctgacccaggtcgtctggctctggctgggtacgcagagcttcgtcttccagcgcgaagagatagcgcagttggcgcggcagtacgctgggctggaccacgagctggccttctctcgtctgatcgtggagctgcggcggctgcacccaggccacgtgctgcccgacgaggagctgcagtgggtgttcgtgaatgcgggtggctggatgggcgccatgtgccttctgcacgcctcgctgtccgagtatgtgctgctcttcggcaccgccttgggctcccgcggccactcggggcgctactgggctgagatctcggataccatcatctctggcaccttccaccagtggagagagggcaccaccaaaagtgaggtcttctacccaggggagacggtagtacacgggcctggtgaggcaacagctgtggagtgggggccaaacacatggatggtggagtacggccggggcgtcatcccatccaccctggccttcgcgctggccgacactgtcttcagcacccaggacttcctcaccctcttctatactcttcgctcctatgctcggggcctccggcttgagctcaccacctacctctttggccaggacccttgaccagccaggcctgaaggaagacctgcggatagacaggagcgggcaggcccgcacatatccacttgctggagcccatgtttacagacagggacatacaccatgcagatcctgagttcctgctgtatgagcagggatatccatgcttatgtatccaaacacagagacccatgggaacaaatgagacacatatagatactgagacctgtgtgtacagtaggaccatgcactcacacccatctggagagggagcccccggtataccaagggagccagttgtgttcagacacacacatcacagcttgactcactaactgaggcctttccatagctccacagcttcccacctcctccccaccaaaccggggttctagagttaaggatgggggagggtattatactgcctcagtctgactcctcaacccagcagcaatttgaggggatgagggggaagaggagctgccttttggaggcccccttcacctgcagctatgatgcccttccccttctcccctgtcctcaccatatgccttatccccattctactcccctgctatgcaagtgcccctgtggcttgtccccaaccccctcagcaacaaagctcagctggggaacgagagtaatttgaagaatgcttgaagtcagcgtcttccattccagaaagacccccattcttcctttgggggtatgatgtggaagctggtttcagcccaggacccaccactgaggagaggatctagacaggtgggcctaattccaaggggcccttcctggcctggagaaggccttttacacacacacaacacatacacacacacacacacacacacatatcacagttttcacacagcccctgctgcattctctgtccatctgtctgtttctattaataaagatttgttgatctgttcca

SEQ ID NO: 2 - protein, 223 aa, Homo sapiens
MQWAVGRRWAWAALLLAVAAVLTQVVWLWLGTQSFVFQREEIAQLARQYAGLDHELAFSRLIVELRRLHPGHVLPDEELQWVFVNAGGWMGAMCLLHASLSEYVLLFGTALGSRGHSGRYWAEISDTIISGTFHQWREGTTKSEVFYPGETVVHGPGEATAVEWGPNTWMVEYGRGVIPSTLAFALADTVFSTQDFLTLFYTLRSYARGLRLELTTYLFGQDP

SEQ ID NO: 3 - DNA, 7895 bp, Homo sapiens
  CDS (116)..(3049)
gctgcctagtcagttctgcgtccgctgaggctcggtcaccgcctcgctgtcgtcgcggcgcccccgccccgtcctctgtccgtaccgcccccggagccagggccgagtcctcgccatgccggcccggcggctgctgctgctgctgacgctgctgctgcccggcctcgggatttttggaagtaccagcacagtgacgcttcctgaaaccttgttgtttgtgtcaacgctggatggaagtttgcatgctgtcagcaagaggacaggctcaatcaaatggactttaaaagaagatccagtcctgcaggtcccaacacatgtggaagagcctgcctttctcccagatcctaatgatggcagcctgtatacgcttggaagcaagaataatgaaggcctgacgaaacttccttttaccatcccagaattggtgcaggcatccccatgccgaagttcagatggaatcctctacatgggtaaaaagcaggacatctggtatgttattgacctcctgaccggagagaagcagcagactttgtcatcggcctttgcagatagtctctgcccatcaacctctcttctgtatcttgggcgaacagaatacaccatcaccatgtacgacaccaaaacccgagagctccggtggaatgccacctactttgactatgcggcctcactgcctgaggacgacgtggactacaagatgtcccactttgtgtccaatggtgatgggctggtggtgactgtggacagtgaatctggggacgtcctgtggatccaaaactacgcctcccctgtggtggccttttatgtctggcagcgggagggtctgaggaaggtgatgcacatcaatgtcgctgtggagaccctgcgctatctgaccttcatgtctggggaggtggggcgcatcacaaagtggaagtacccgttccccaaggagacagaggccaagagcaagctgacgcccactctgtatgttgggaaatactctaccagcctctatgcctctccctcaatggtacacgagggggttgctgtcgtgccccgcggcagcacacttcctttgctggaagggccccagactgatggcgtcaccattggggacaagggggagtgtgtgatcacgcccagcacggacgtcaagtttgatcccggactcaaaagcaagaacaagctcaactacttgaggaattactggcttctgataggacaccatgaaaccccactgtctgcgtctaccaagatgctggagagatttcccaacaatctacccaaacatcgggaaaatgtgattcctgctgattcagagaaaaagagctttgaggaagttatcaacctggttgaccagacttcagaaaacgcacctaccaccgtgtctcgggatgtggaggagaagcccgcccatgcccctgcccggcccgaggcccccgtggactccatgcttaaggacatggctaccatcatcctgagcaccttcctgctgattggctgggtggccttcatcatcacctatcccctgagcatgcatcagcagcagcagctccagcaccagcagttccagaaggaactggagaagatccagctcctgcagcagcagcagcagcagctgcccttccacccacctggagacacggctcaggacggcgagctcctggacacgtctggcccgtactcagagagctcgggcaccagcagccccagcacgtcccccagggcctccaaccactcgctctgctccggcagctctgcctccaaggctggcagcagcccctccctggaacaagacgatggagatgaggaaaccagcgtggtgatagttgggaaaatttccttctgtcccaaggatgtcctgggccatggagctgagggcacaattgtgtaccggggcatgtttgacaaccgcgacgtggccgtgaagaggatcctccccgagtgttttagcttcgcagaccgtgaggtccagctgttgcgagaatcggatgagcacccgaacgtgatccgctacttctgcacggagaaggaccggcaattccagtacattgccatcgagctgtgtgcagccaccctgcaagagtatgtggagcagaaggactttgcgcatctcggcctggagcccatcaccttgctgcagcagaccacctcgggcctggcccacctccactccctcaacatcgttcacagagacctaaagccacacaacatcctcatatccatgcccaatgcacacggcaagatcaaggccatgatctccgactttggcctctgcaagaagctggcagtgggcagacacagtttcagccgccgatctggggtgcctggcacagaaggctggatcgctccagagatgctgagcgaagactgtaaggagaaccctacctacacggtggacatcttttctgcaggctgcgtcttttactacgtaatctctgagggcagccacccttttggcaagtccctgcagcggcaggccaacatcctcctgggtgcctgcagccttgactgcttgcacccagagaagcacgaagacgtcattgcacgtgaattgatagagaagatgattgcgatggatcctcagaaacgcccctcagcgaagcatgtgctcaaacacccgttcttctggagcctagagaagcagctccagttcttccaggacgtgagcgacagaatagaaaaggaatccctggatggcccgatcgtgaagcagttagagagaggcgggagagccgtggtgaagatggactggcgggagaacatcactgtccccctccagacagacctgcgtaaattcaggacctataaaggtggttctgtcagagatctcctccgagccatgagaaataagaagcaccactaccgggagctgcctgcagaggtgcgggagacgctggggtccctccccgacgacttcgtgtgctacttcacatctcgcttcccccacctcctcgcacacacctaccgggccatggagctgtgcagccacgagagactcttccagccctactacttccacgagcccccagagccccagcccccagtgactccagacgccctctgagcgagggcggcccctctgttctggtggccccagctgtgactgagggcctggtcaccacaattagagcttgatgcctcccggctttgcagggagaccaggcttcccaaaccaagtgccttgagctgcctgctctgcagcccacagaggacagtgctgaccccaggaagtgggagaagtggcccctcgtgacctacagggaactgggaagatgctggccccaaaagccttacggtcatgatgtctgcaaaggagggcctcagagacagcgcgagtagcacccccagccatctactggataaacttgcttcagactttttaaattcctgcttaatgtcagtctacaggcctttcaggaagggagaggagggaatcgtacattttgcttgcgtgctgggacagctaggctgagatgcaccaagtacagccttcactggagaccggaattgagaggtgggggatgctgaggagggggaggacggagttcagagggtgtcgtcctgcagtgtgagatttctcattgatcacagatgtgcccagagtagcccaggtcactgttaactagtgtttctgcagaggcagcaggagccatgagcatgaggtgtggcattagggactggtcagctatgcatgctggcaggtggggttgtgtctgcaggtctcagaaatgaagaggctgctctgttctggaggcagccgtggcccagtgccagtggccagaacagtggcctttggtgggtgtgtcccgggccatctcggggtggtgctcaggagcgcctggggcaagaggtaaagagttccctggccttcaaggagagcagcgaagacccagacaggggccagccttcaggaccagagggaggccgccgaatgggaccctcctggtcaccaggagaaagccctgggccagcgagtaggcagtcaaactccttcgtccccaaggccggtggaacaagaggctcgtggtgagtcagggccagggtgggtggccaaggccagggtcaccgtgtgcttcatgggccagcttttttgtttttcttggcaaattttaataactatattttgattatactgtagaatgctatgtcagcataagtaagctaaacttgaagctttcttgtgaagaataaatgcaagatagaatacatcttctattttttgtggtaccaaaaatcaccatcccctcaagagtgttcatgtatagaacattctctaatgctgaagagtaaaacattatagcaacactatgtaaatgtattgaacagtatcaaagaaatagtctctaaattgtttgtaccatatttttttttctaaacttaacataatttttagctttagtttcagtcaaaactttgtcttttctctcccgagagccttagaggttaaaatgcaatcagcctaccgtgtaaggagatgttgtccatgtactttctccagccagttgggggatcattgcagctcaggcctggtgaactcagagattccattcagtattaagaatgggattgttgaattttactcacagagaaatcactgtttcttcatgttgtaagatgttttctgtttgtgtatttgtatcatggttactcatcaaaagctctcattctgcctttgtagaattcagttcccttcctttcatcatagctaaagtgacttttttccctactattaacgtgatcctacatccttaaatctcatcgattacctcacttaggccttggaaccttggcccttggtcggtgtccttggcgtcttctaagcaaggctgtgcgttgttcagaaacgtggccagaccgcatttcctgctgctcccatgccgcatgccaggtggcctgagacagagctccccatacggctgcaaggtgctttacctgtgggctttggcagtaacccaagagaggatcagaaggtggagaaggtgccacgagtgatttaacaggcctgccacagggagtgcccccagcccagctcgttctcagcacaggttttctctttgggagtacccaggtgatttctagtgacccaattttgtgtcatctccctgttttagccccacttgcctagagacaactgtttccgatgccttttctgcttatcatactagtttctaaccacgcagatttctcaaaatcatttattcaatgtattttatttgagcacttagtgtattgagctaggcaggatatagggtgccggagatacagcgatgaacaagacaggcaaaacttctgctttcctaaaacttgtgatgagagagaacaataaaaaagtgttgctgccacaaagaaaccaaagtgtgtggggaagggcgcgtgtttgcggtttacatctcttctgtcccagaatcacagggatctggtccggtcacctctggtctttcctcttagtcgcctttaggagccctggttcccgtccatcctctggggggtttgtttgaagagatctcgtgtgggtacttgtcatgaaaacaccttgggcatcatctggtgtatccagttctagtctcgagaattctggtttcccactgtgctcagcaagtggaaagttcttttcaggccagaacagctctgcaccatcacatatcgtgttgcggcttagctgtttggtctgtagttcaggttatgggacttctccaatcctggaaggctgttgagctttttagaagtactgtacgctatcttcaagatggagcttggtcacatctgttaggaatccaaaggacactatgacttatttaaatcttgtcttactaaacctctcttgggcacgtgtgccagaatttctcttgttgcttcttgagtctttttaatttcagtgttttttcgtttgttttttgtttttttgagacagagtctcgctctgtcacccaggctggagtgcagtggcacgacctcagctcactgcaacctccgcctccttggttcaagcaattctcctgcctcagcctcccgagtagctgggattacaggtgtgtgccaccacgctcggctaattttttatattttttttagtagagacggggtttccccatgttaggcaggatggtctcgatctcctaacctcgtgatccgcctgcctcagcctcccaaagtgctgggattacaggcttgagccaccgcacccggcctaatctcagtttttgaagtgctccacaagtcattaggcaccaaaacattttcacctggggaacactggcatttccctgattagctgtgaagcaatctagtggctaagtgtgaaatcctgggtgcgcaggtgttctcactcccgccgtgttctcagtgcagtggtggtcagaggcccttccaaggagacatcactctgatcagttacagatagatgttctggaagatctgcaggtgagtagatccagcagagtttcttcccaccaactctagaagaaagggccttatcagagttgaccctgagcctttggtaaggttttgtgtgcatgcgattcagttatctttggcaattcttttcttgctgcagtgagagattaattggttgctgatcaaaccgttcatgcagatggggggacctttggattgtacggctttctcctcttggctgctttcttttcaggaagttggactttggccaggtttggctttcccagagccgttcctttctctgtcctttccttgggtcctcatggtgtgcccattggatccttggccttgtgatcctctgggaactgggggccagttccacttttgcagccttctgtgctggaagagaagcccagcgccctggaaggagcctctttaagtcccccatgtcgctttctctctctgctcttttagtgtctgagattgcctttctttgaatttcccagtgtttcttttccttgtcccttccctcaccaacctggagttattttggttgactatgtcctggctttggcttctcctggcaggaagtcatcaggcatcctctccaggtgagccgaaattccaccctcccaggttggacatcatcttttaaacccaatggtctactcccctccttctttatgaaacagtgatttcccgtgagtaactctggttctgattttttgtaccggcgcttaaattctttctgtagacgttggaaagccacaaagaacgtgactgcagtgagcctcccactggagcagccttaaccaacactttggccaaagcccccccacctcccctgtgtactgtgtgtgtgtttggtggatacagtattccttttcagtgtccctaaagctgtgatggggagtccccacttacctagaaagcattaccagtcacctactctgcattctcagatgtaaaccttgtgtagtgttctttttgcaatgacctatttatttaacctatttatatttatttaatttttactctgaaatgtatccagttacaattgtacttgcttaaagcacatcagatttgttttggacaacacccttgaccattttaaaactggaaaagtgatactgtatccttccatgggatggatgctttacagtagtcttattattaaagggtgattaatttggtcggggtaaaatgttaatttttaggtgatttttaagaattctgtgccattatgtcttctgtgtggatggttaattgtttaattagtacgtgttaattgtgtgatacagtcttctttgtggaacccaaaatcctctttttagctttatattttataaactgccagattgtacaacttttatgtgcatttttaaagcttgaagacatgagggtcattatctaagttaaacagcctatttttgtgcctcctgtacagttttataattctgctgatggcggcatcttatgtcgagccaaccacaataaaggtagttttagattttgga

SEQ ID NO: 4 - protein, 977 aa, Homo sapiens
MPARRLLLLLTLLLPGLGIFGSTSTVTLPETLLFVSTLDGSLHAVSKRTGSIKWTLKEDPVLQVPTHVEEPAFLPDPNDGSLYTLGSKNNEGLTKLPFTIPELVQASPCRSSDGILYMGKKQDIWYVIDLLTGEKQQTLSSAFADSLCPSTSLLYLGRTEYTITMYDTKTRELRWNATYFDYAASLPEDDVDYKMSHFVSNGDGLVVTVDSESGDVLWIQNYASPVVAFYVWQREGLRKVMHINVAVETLRYLTFMSGEVGRITKWKYPFPKETEAKSKLTPTLYVGKYSTSLYASPSMVHEGVAVVPRGSTLPLLEGPQTDGVTIGDKGECVITPSTDVKFDPGLKSKNKLNYLRNYWLLIGHHETPLSASTKMLERFPNNLPKHRENVIPADSEKKSFEEVINLVDQTSENAPTTVSRDVEEKPAHAPARPEAPVDSMLKDMATIILSTFLLIGWVAFIITYPLSMHQQQQLQHQQFQKELEKIQLLQQQQQQLPFHPPGDTAQDGELLDTSGPYSESSGTSSPSTSPRASNHSLCSGSSASKAGSSPSLEQDDGDEETSVVIVGKISFCPKDVLGHGAEGTIVYRGMFDNRDVAVKRILPECFSFADREVQLLRESDEHPNVIRYFCTEKDRQFQYIAIELCAATLQEYVEQKDFAHLGLEPITLLQQTTSGLAHLHSLNIVHRDLKPHNILISMPNAHGKIKAMISDFGLCKKLAVGRHSFSRRSGVPGTEGWIAPEMLSEDCKENPTYTVDIFSAGCVFYYVISEGSHPFGKSLQRQANILLGACSLDCLHPEKHEDVIARELIEKMIAMDPQKRPSAKHVLKHPFFWSLEKQLQFFQDVSDRIEKESLDGPIVKQLERGGRAVVKMDWRENITVPLQTDLRKFRTYKGGSVRDLLRAMRNKKHHYRELPAEVRETLGSLPDDFVCYFTSRFPHLLAHTYRAMELCSHERLFQPYYFHEPPEPQPPVTPDAL

SEQ ID NO: 5 - RNA, 22 nt, Homo sapiens
uugcauagucacaaaagugauc

SEQ ID NO: 6 - RNA, 22 nt, Homo sapiens
cguguucacagcggaccuugau

SEQ ID NO: 7 - RNA, 23 nt, Homo sapiens
ugagugugugugugugagugugu

SEQ ID NO: 8 - RNA, 21 nt, Homo sapiens
cacacacacacacacacguau

SEQ ID NO: 9 - RNA, 23 nt, Homo sapiens
cugggcccgcggcgggcgugggg

SEQ ID NO: 10 - RNA, 19 nt, Homo sapiens
gcacgugaauugauagaga

SEQ ID NO: 11 - RNA, 19 nt, Homo sapiens
ucucuaucaauucacgugc